(19) United States
(12) Patent Application Publication
(10) Pub. No.: US 2024/0336836 A1
(43) Pub. Date: Oct. 10, 2024

(54) METAL IRIDIUM COMPLEX AND ELECTROLUMINESCENT DEVICE

(71) Applicant: GUANGDONG AGLAIA OPTOELECTRONIC MATERIALS CO., LTD., Foshan (CN)

(72) Inventors: Shaofu CHEN, Foshan (CN); Liangliang YAN, Foshan (CN); Lei DAI, Foshan (CN); Lifei CAI, Foshan (CN)

(73) Assignee: GUANGDONG AGLAIA OPTOELECTRONIC MATERIALS CO., LTD., Foshan (CN)

(21) Appl. No.: 18/658,432

(22) Filed: May 8, 2024

Related U.S. Application Data
(63) Continuation of application No. PCT/CN2022/123702, filed on Oct. 4, 2022.

(30) Foreign Application Priority Data
Nov. 9, 2021 (CN) 202111317115.X

(51) Int. Cl. C09K 11/06; H10K 50/12

(52) U.S. Cl. CPC C09K 11/06; H10K 50/12; C09K 2211/1033; C09K 2211/1037

(57) ABSTRACT

The present invention relates to a metal iridium complex and an electroluminescent device. The metal iridium compound has a general formula of Ir(La)(Lb)(Lc), wherein La is a structure shown in formula (1), and Lb is a structure shown in formula (2). The compound provided in the present invention can be used in organic light-emitting devices, particularly as a red luminescent phosphorescent material, and has the potential to be applied to the AMOLED industry, particularly for display, lighting, and automobile taillights.

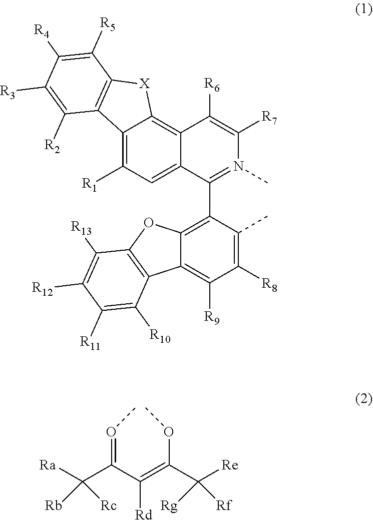

### CROSS-REFERENCE TO RELATED APPLICATIONS

This application is a bypass continuation of International Application No.: PCT/CN2022/123702 filed on Oct. 4, 2022, which claims the benefit of Chinese Patent Application No. 202111317115X, filed on Nov. 9, 2021, the entire contents of which are incorporated by reference in their entirety.

### TECHNICAL FIELD

The present disclosure relates to the technical field of organic electroluminescence, especially to an organic luminescent material, in particular to a metal iridium complex and use thereof in an organic electroluminescent device.

### BACKGROUND

At present, as a new generation of display technology, organic electroluminescent devices (OLEDs) have attracted more and more attention in both display and lighting technologies, and their application prospects are very broad. However, with respect to market application requirements, the performance of OLED devices in terms of luminous efficiency, driving voltage, service life, etc., still needs to be further strengthened and improved.

Generally, the basic structure of OLED devices is that various organic functional material thin films with different functions are sandwiched between metal electrodes, just like a sandwich structure. Driven by current, holes and electrons are injected from an anode and a cathode, respectively. After moving for a certain distance, the holes and electrons are recombined in a luminous layer and released in the form of light or heat, thus producing the luminescence of the OLEDs. However, organic functional materials are the core components of organic electroluminescent devices, and their thermal stability, photochemical stability, electrochemical stability, quantum yield, film-forming stability, crystallinity, color saturation, etc., are all main factors that affect the performance of the devices.

Generally, organic functional materials include fluorescent materials and phosphorescent materials. Fluorescent materials are usually organic small molecular materials, which can only utilize 25% singlet state to emit light, so the luminous efficiency is relatively low. Whereas, due to spin-orbit coupling caused by heavy-atom effect, phosphorescent materials can utilize the energy of 75% triplet excitons in addition to the 25% singlet state, so the luminous efficiency can be improved. However, compared with fluorescent materials, the research of phosphorescent materials started late, and their thermal stability, lifetime, color saturation, etc., all need to be improved, which is a challenging task. Various compounds have been developed as phosphorescent materials. For example, China patent CN 107973823 discloses a class of quinoline-based iridium compounds; however, the color saturation and device performance of these compounds, especially in terms of luminous efficiency and device lifetime, all need to be improved. China patent CN 106459114 discloses a class of iridium compounds coordinated by β-diketone dentate ligands; however, these compounds have a high sublimation temperature and poor color saturation. In particular, the device performance, especially in terms of luminous efficiency and device lifetime, is not ideal and needs to be further improved. Moreover, China patent CN 111377969 discloses a class of iridium complexes of dibenzofuran isoquinoline biisoquinoline

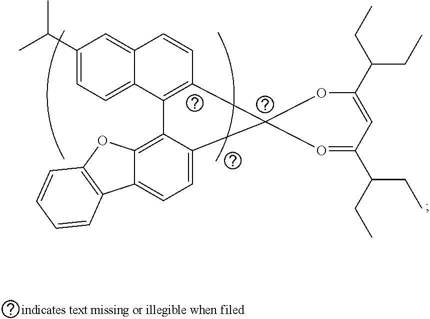

however, the device performance of this material, especially in terms of color saturation, cannot meet the display color gamut requirements of BT2020 and needs to be further improved in order to meet the demand of the rapidly developing market for OLED luminescent materials. China patent CN 108290914 A discloses a structure of quinolinophenyl-fused five-membered heterocyclic ring

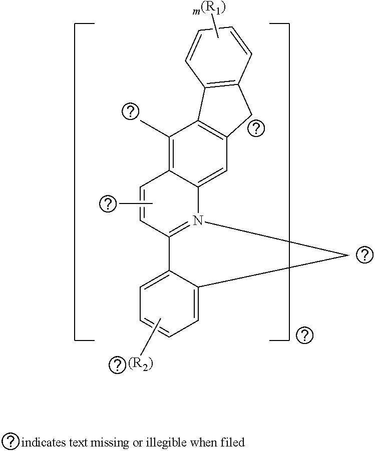

as a red luminescent material; however, the device color coordinates of this material cannot meet the requirements of wide color gamut, and the patent neither discloses nor teaches that the connection and combination mode of the disclosure can bring about improved device performance and emission wavelength. China patent document CN 111848689 A discloses a structure of isoquinolobenzofuran

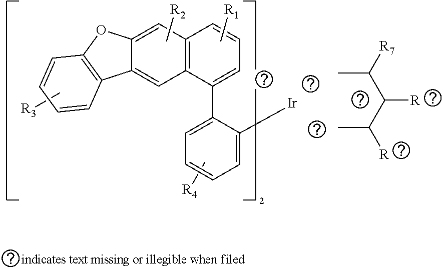

as a red-light emitter. This type of material shows excellent device efficiency and lifetime. However, from the data of the device described in the patent, it can be seen that the compound described in the disclosure still cannot meet the deeper red color development requirements of BT2020. Although color coordinates can be adjusted to CIEx of about 0.70 by means of the microcavity effect of top emission, there is still room for improvement in device efficiency and lifetime to meet the market demand.

### SUMMARY

The present disclosure has been completed in order to solve the above problems, and an objective thereof is to provide a high-performance organic electroluminescent device and a novel material that can realize such an organic electroluminescent device.

In order to achieve the above-mentioned objective, the present inventors have repeatedly conducted in-depth studies, and as a result, found that high-performance organic electroluminescent devices can be obtained by using metal iridium complexes comprising the following formulas (1) and (2) as ligands.

The metal iridium complex has a general formula of Ir(La)(Lb)(Lc), wherein La is a structure shown in formula (1), and Lb is a structure shown in formula (2). The complex provided by the present disclosure has the advantages of a good optical and electrical stability, a high luminous efficiency, a long lifetime, a high color saturation, etc., can be used in organic light-emitting devices, particularly as a red luminescent phosphorescent material, and has the potential to be applied to the Active-matrix organic light-emitting diode (AMOLED) industry, particularly for display, lighting and automobile taillights.

A metal iridium compound having a general formula of Ir(La)(Lb)(Lc), wherein La is a structure shown in formula (1),

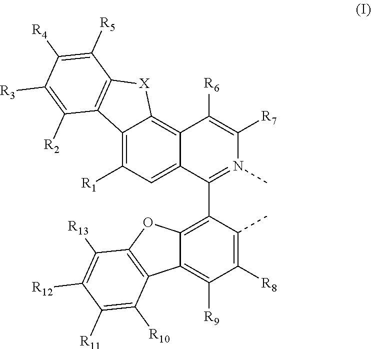

wherein a dotted line represents a position of connection to the metal Ir;
wherein X is O, S, Se, C(R₀)₂, or Si(R₀)₂;
wherein R₀-R₁₃ are each independently hydrogen, deuterium, halogen, cyano, substituted or unsubstituted C1-C10 alkyl, substituted or unsubstituted C1-C10 heteroalkyl, substituted or unsubstituted C3-C20 cycloalkyl, substituted or unsubstituted C3-C20 heterocycloalkyl, substituted or unsubstituted C2-C10 alkenyl, substituted or unsubstituted C2-C10 alkynyl, substituted or unsubstituted C6-C30 aryl, substituted or unsubstituted C2-C30 heteroaryl, substituted or unsubstituted tri(C1-C10)alkylsilyl, substituted or unsubstituted tri(C6-C12)arylsilyl, substituted or unsubstituted di(C1-C10)alkylmono(C6-C30)arylsilyl, or substituted or unsubstituted mono(C1-C10)alkyldi(C6-C30)arylsilyl, or two adjacent groups of R₁₀-R₁₃ are connected with each other to form an aliphatic ring;
wherein R₈ is not hydrogen, deuterium, halogen, or cyano;
wherein the heteroalkyl, heterocycloalkyl, and heteroaryl contain at least one O, N or S heteroatomn;
wherein the substitution is substitution with deuterium, F, Cl, Br, C1-C6 alkyl, C3-C6 cycloalkyl, C1-C6 alkylamino, cyano, isocyano or phosphine group, and a number of substitutions is from mono-substitution to the maximum number of substitutions;
wherein Lb is a structure shown in formula (2),

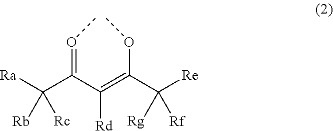

wherein a dotted line represents a position of connection to the metal Ir;
wherein Ra-Rg are each independently hydrogen, deuterium, halogen, substituted or unsubstituted C1-C10 alkyl, substituted or unsubstituted C3-C20 cycloalkyl, substituted or unsubstituted C1-C10 heteroalkyl, or substituted or unsubstituted C3-C20 heterocycloalkyl, or Ra, Rb, and Rc are connected in pairs to form an aliphatic ring, and Re, Rf, and Rg are connected in pairs to form an aliphatic ring;
wherein the heteroalkyl and heterocycloalkyl contain at least one 0, N or S heteroatom;
wherein the substitution is substitution with deuterium, F, Cl, Br, C1-C4 alkyl, C1-C4 alkoxy, C3-C6 cycloalkyl, C1-C4 alkylamino, cyano, isocyano or phosphine group;
wherein Lc is a single anionic bidentate ligand, and Lc is different from Lb and is not an OO-type ligand;
wherein Lc and La are the same or different, the difference means that a mother nucleus structure is different, or the mother nucleus structure is the same but substituents are different, or the mother nucleus structure is the same and the substituents are the same but positions of the substituents are different;
wherein La, Lb, and Lc are connected in pairs or in triplets to form a multidentate ligand.

In an embodiment, X is O, S, C(R₀)₂, or Si(R₀)₂, wherein R₀ is substituted or unsubstituted C1-C6 alkyl.

In an embodiment, at least one of R₂-R₇ is not H.

In an embodiment, at least one of R₁-R₇ is F, cyano, substituted or unsubstituted C1-C6 alkyl, or substituted or unsubstituted C3-C10 cycloalkyl, wherein the substitution is substitution with deuterium, F, C1-C5 alkyl, or C3-C6 cycloalkyl.

In an embodiment, R₈ is substituted or unsubstituted C1-C6 alkyl, or substituted or unsubstituted C3-C6 cycloalkyl, wherein the substitution is substitution with deuterium, F, C1-C5 alkyl, or C3-C6 cycloalkyl.

In an embodiment, R₈ is methyl or deuterated methyl.

In an embodiment, R₉-R₁₃ are hydrogen.

In an embodiment, Lc is different from La.

In an embodiment, Lc is a structure shown in formula (3),

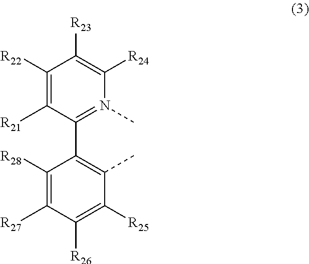

wherein R₂₁-R₂₈ are each independently hydrogen, deuterium, halogen, cyano, hydroxyl, amino, imino, substituted or unsubstituted C1-C10 alkyl, substituted or unsubstituted C1-C10 heteroalkyl, substituted or unsubstituted C3-C20 cycloalkyl, substituted or unsubstituted C2-C10 alkenyl, substituted or unsubstituted C2-C10 alkynyl, substituted or unsubstituted C6-C18 aryl, substituted or unsubstituted C2-C17 heteroaryl, substituted or unsubstituted tri(C1-C10)alkylsilyl, substituted or unsubstituted tri(C6-C12)aryl-silyl, substituted or unsubstituted di(C1-C10)alkylmono(C6-C30)arylsilyl, or substituted or unsubstituted mono(C1-C10)alkyldi(C6-C30)arylsilyl;
wherein at least two of R₂₅-R₂₈ are not hydrogen;
wherein an aromatic ring shown in the following formula (4) is formed between at least one group of two adjacent groups of R₂₁-R₂₄;

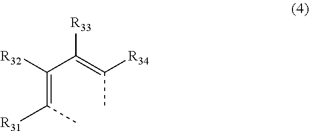

in formula (4),
wherein a dotted line represents a position of connection to a pyridine ring;
wherein R₃₁-R₃₄ are each independently hydrogen, deuterium, halogen, cyano, substituted or unsubstituted C1-C10 alkyl, substituted or unsubstituted C1-C10 heteroalkyl, substituted or unsubstituted C3-C20 cycloalkyl, substituted or unsubstituted C2-C10 alkenyl, substituted or unsubstituted C2-C10 alkynyl, substituted or unsubstituted C6-C18 aryl, substituted or to unsubstituted C2-C17 heteroaryl, substituted or unsubstituted tri(C1-C10)alkylsilyl, substituted or unsubstituted tri(C6-C12)arylsilyl, substituted or unsubstituted di(C1-C10)alkylmono(C6-C30)arylsilyl, or substituted or unsubstituted mono(C1-C10)alkyldi(C6-C30)arylsilyl, or two adjacent groups of R₃₁-R₃₄ are connected with each other to form an aliphatic ring or an aromatic ring;
wherein the heteroalkyl and heteroaryl contain at least one O, N or S heteroatom;
wherein the substitution is substitution with deuterium, F, Cl, Br, C1-C6 alkyl, C3-C6 cycloalkyl, C1-C6 alkylamino, cyano, isocyano or phosphine group, and a number of substitutions is from mono-substitution to the maximum number of substitutions.

An aromatic ring shown in formula (4) is formed between at least one group of two adjacent groups of R₂₁-R₂₃, R₃₁-R₃₄ are each independently hydrogen, deuterium, halogen, cyano, substituted or unsubstituted C1-C6 alkyl, substituted or unsubstituted C1-C6 heteroalkyl, substituted or unsubstituted C3-C10 cycloalkyl, substituted or unsubstituted C6-C10 aryl, or substituted or unsubstituted C2-C10 heteroaryl.

In an embodiment, Lc is one of the following structural formulas, or one of the following structural formulas in which the hydrogen atom(s) is/are partially or completely deuterated or one of the following structural formulas in which the hydrogen atom(s) is/are partially or completely fluorinated,

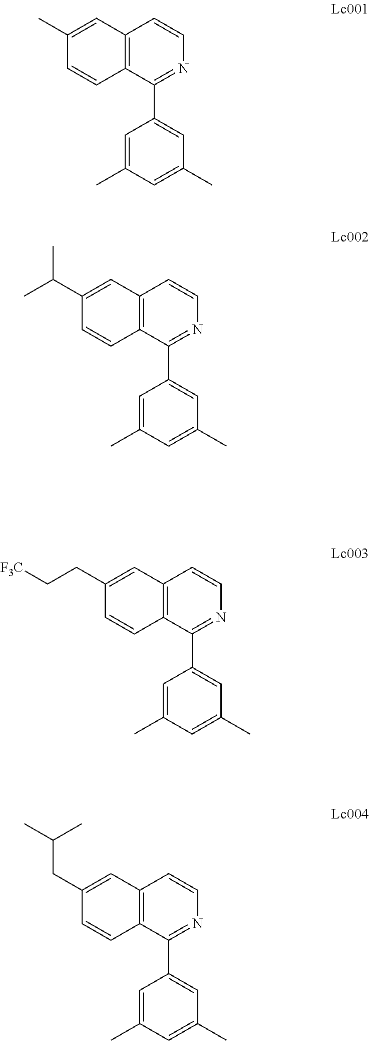

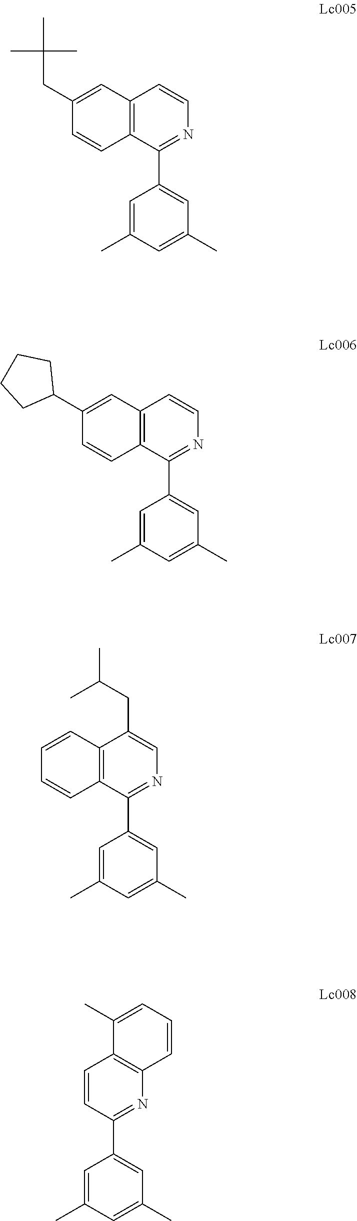

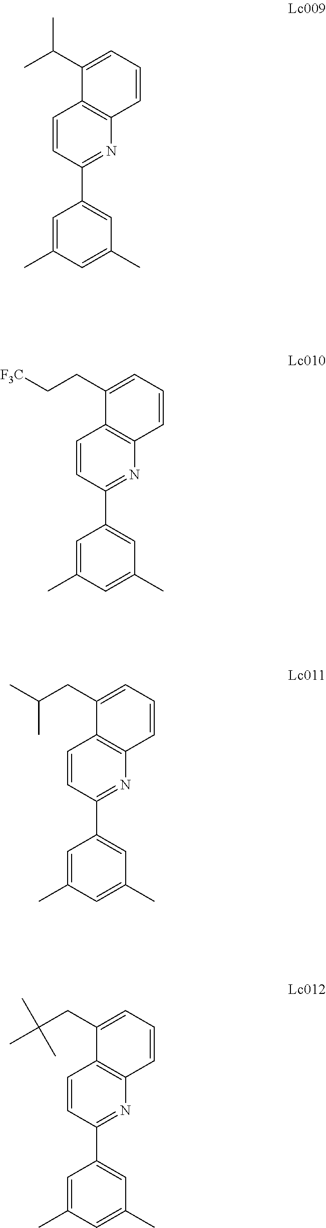 [SMILES: Cc1cc(C)cc(-c2ccc3c(C(C)C)cccc3n2)c1 ; Cc1cc(C)cc(-c2ccc3c(CCC(F)(F)F)cccc3n2)c1 ; Cc1cc(C)cc(-c2ccc3c(CC(C)C)cccc3n2)c1 ; Cc1cc(C)cc(-c2ccc3c(CC(C)(C)C)cccc3n2)c1]

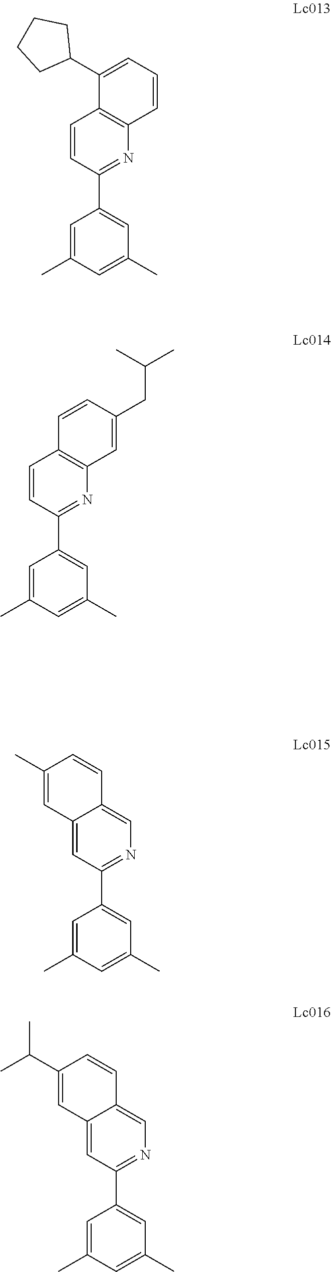

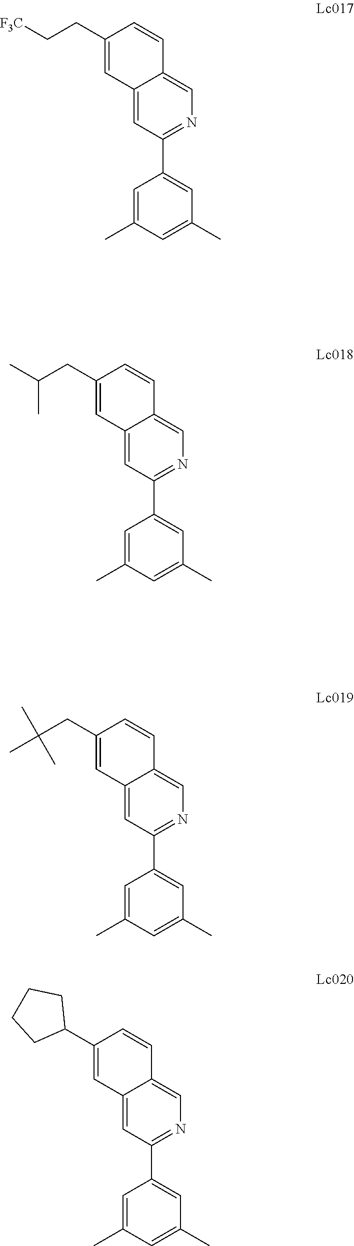

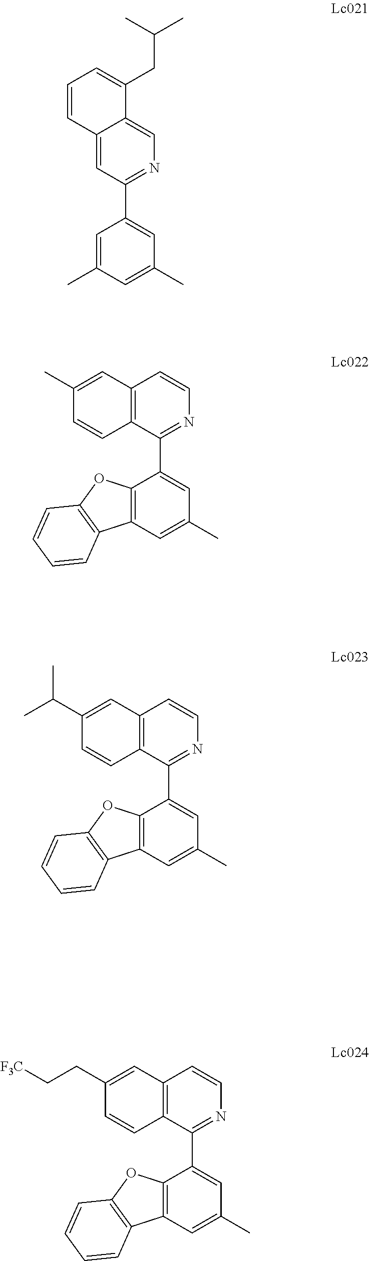

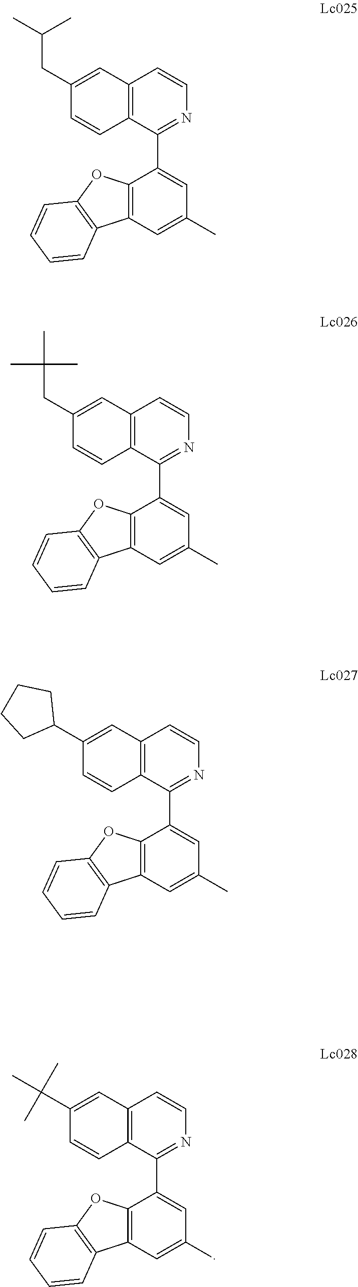

In an embodiment, La is one of the following structural formulas, or one of the following structural formulas in which the hydrogen atom(s) is/are partially or completely deuterated, or one of the following structural formulas in which the hydrogen atom(s) is/are partially or completely fluorinated,

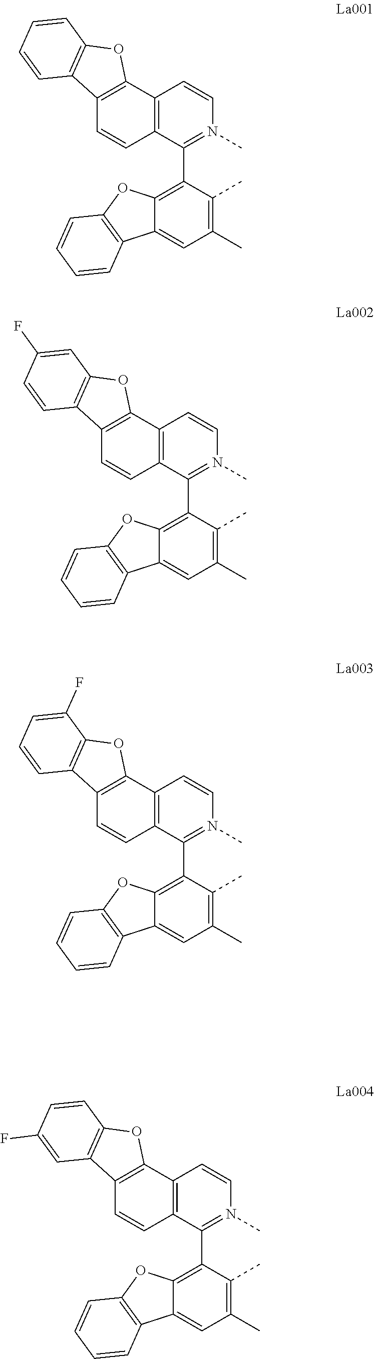

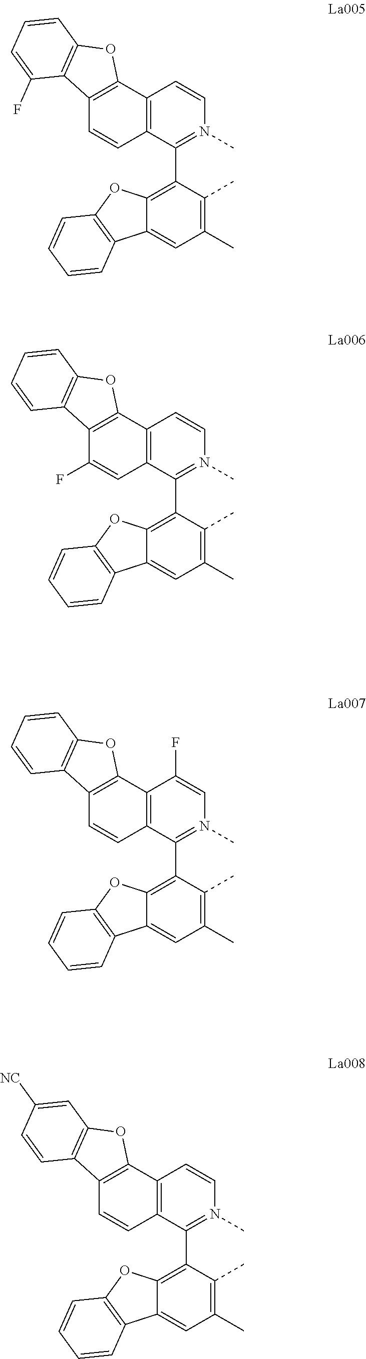

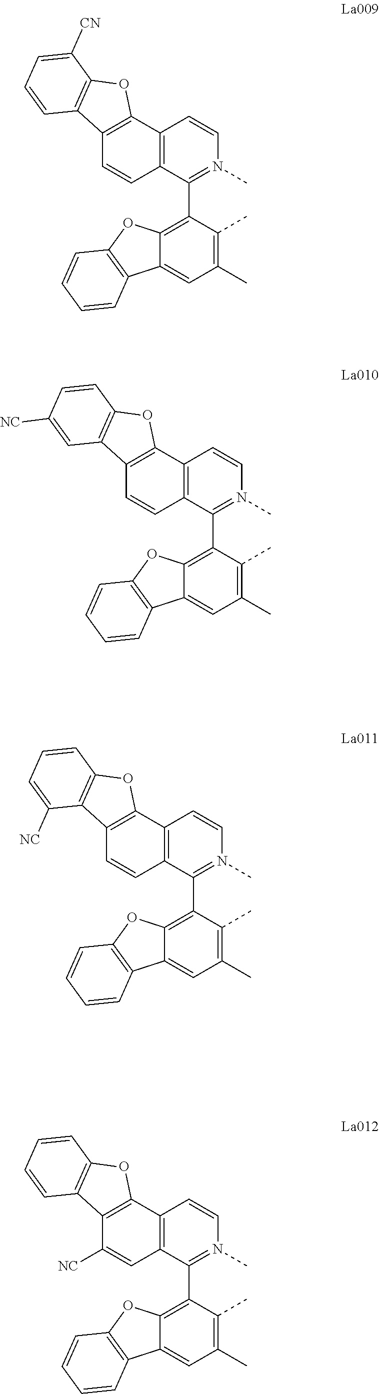

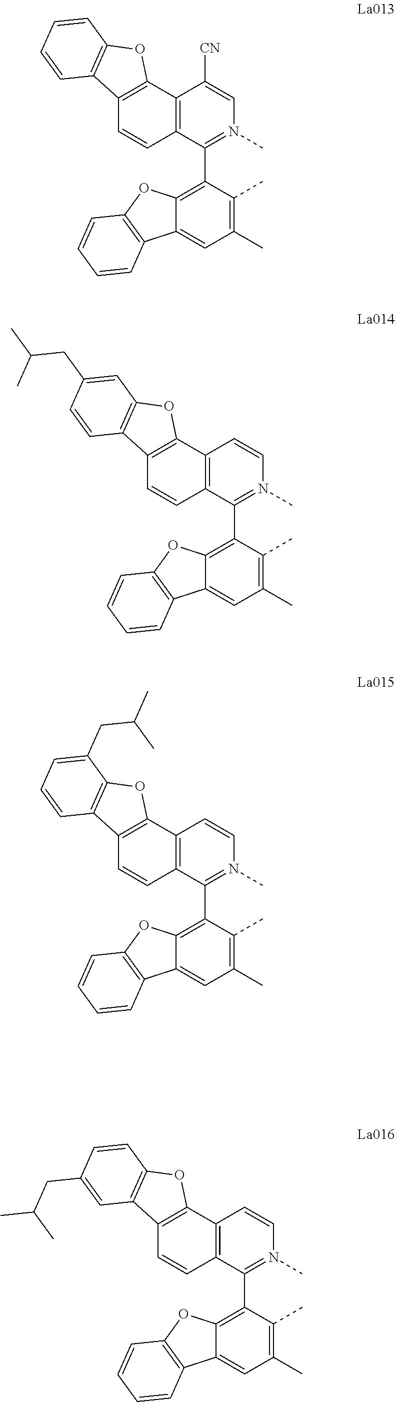

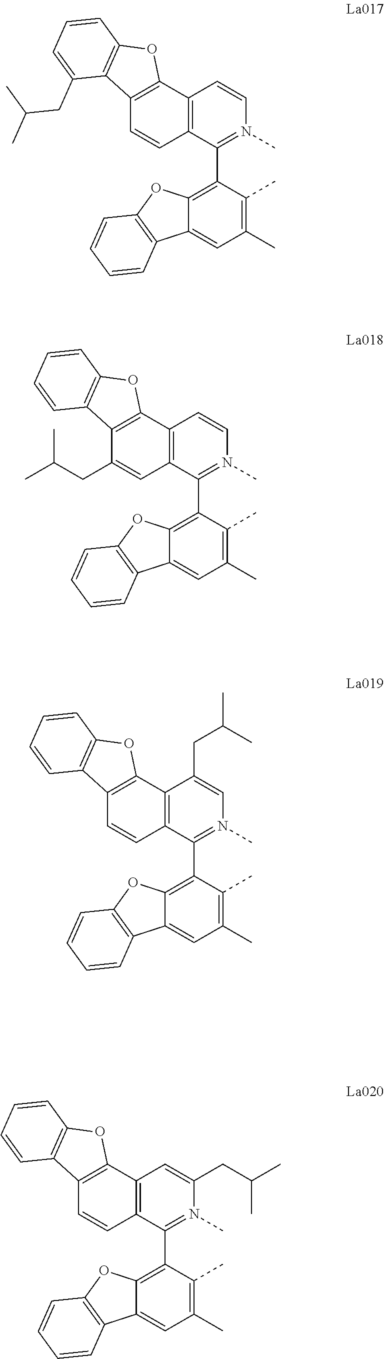

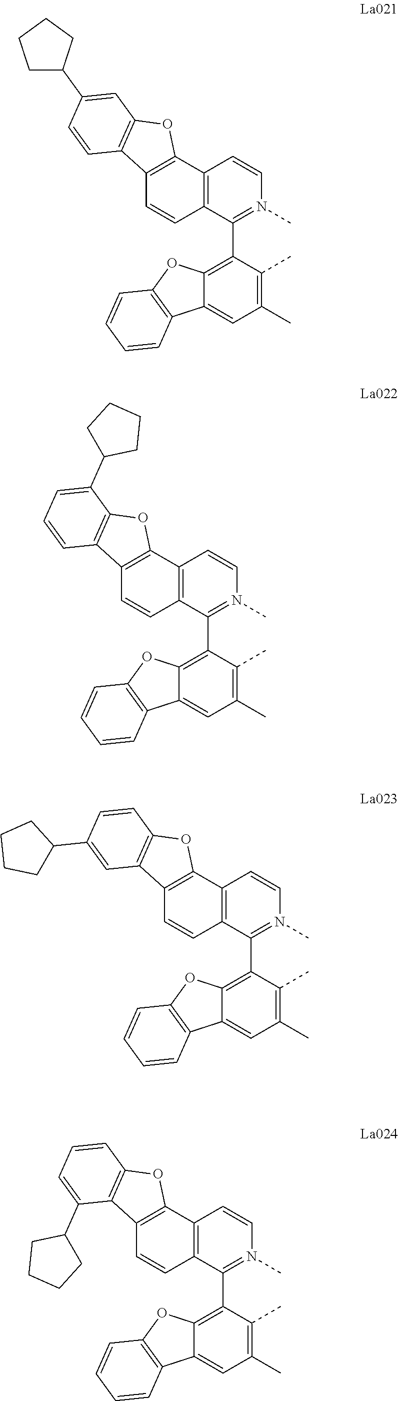

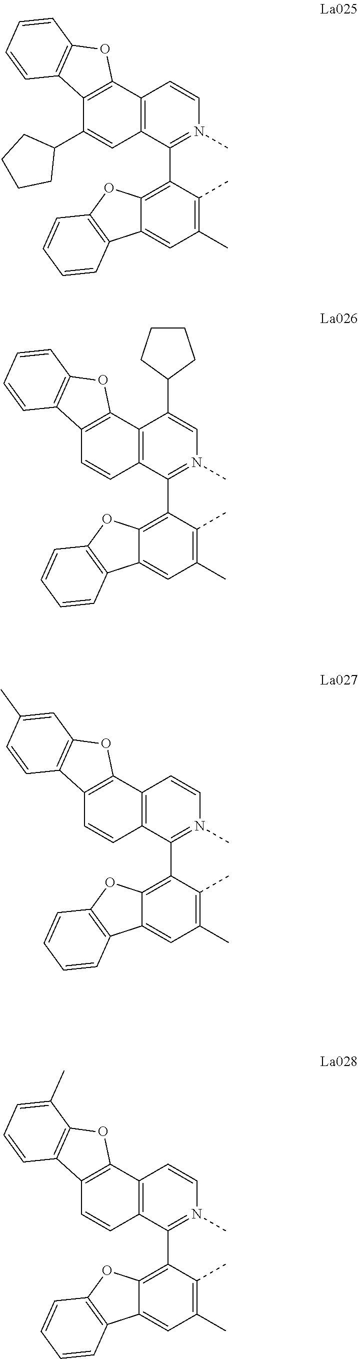

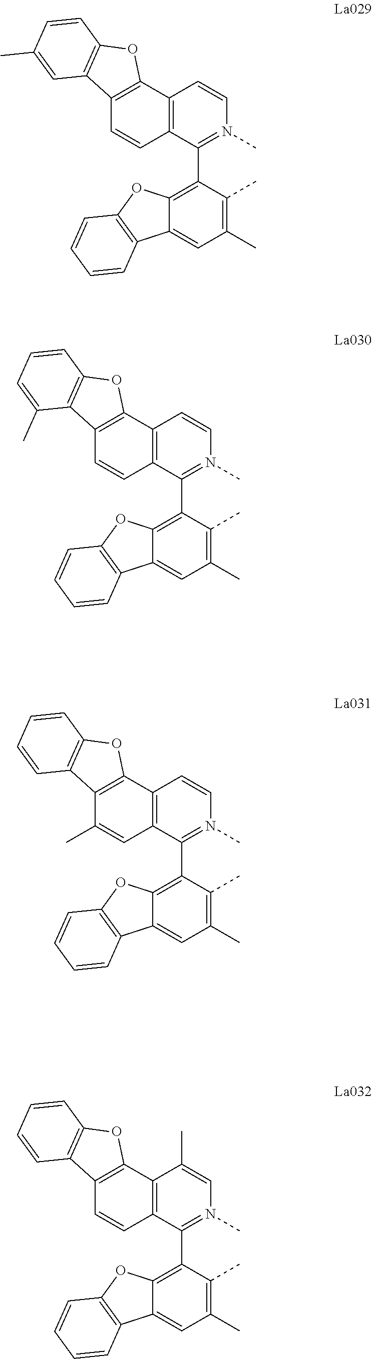

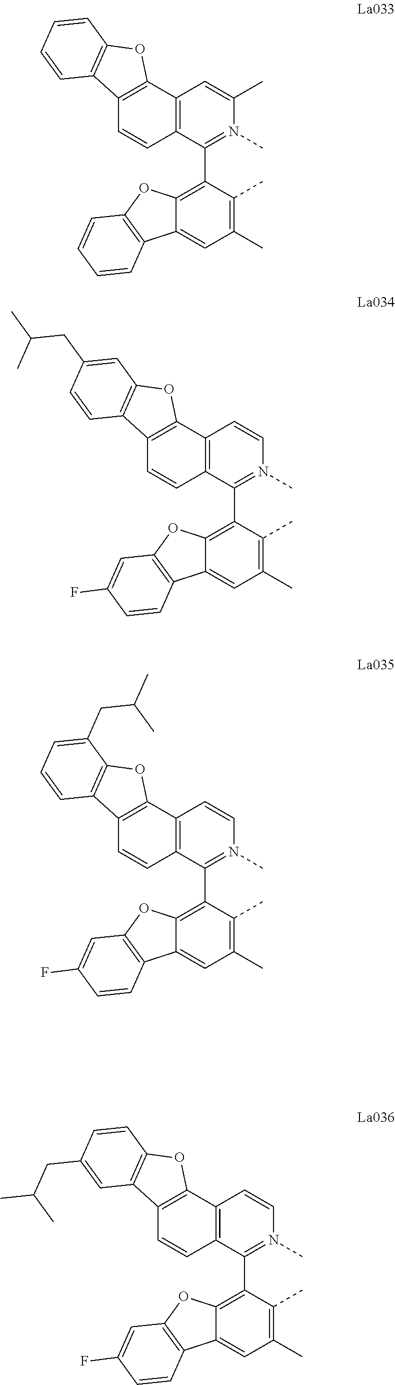

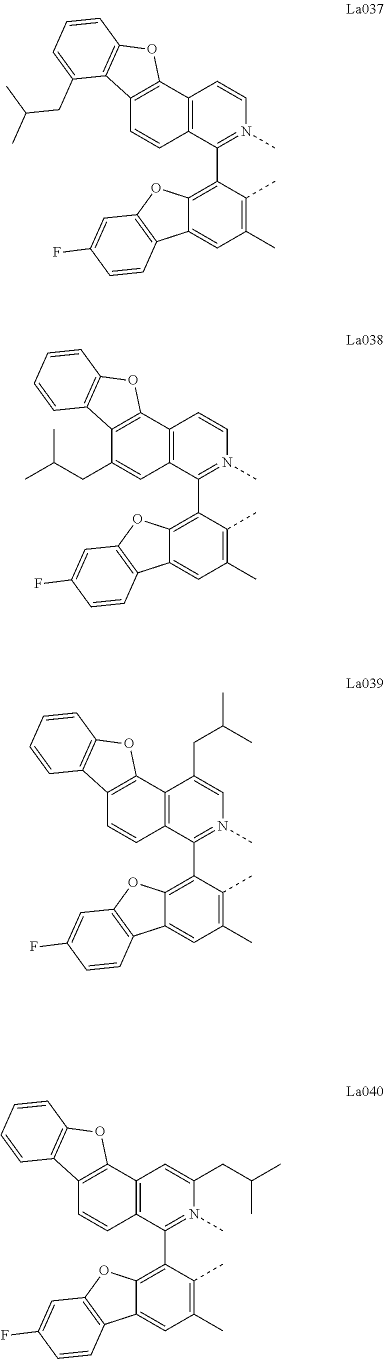

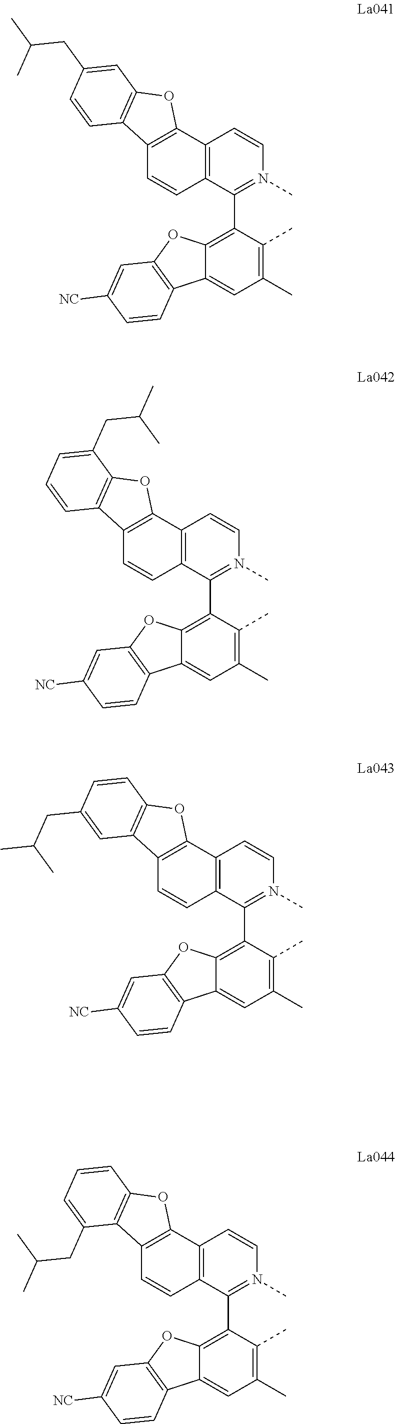

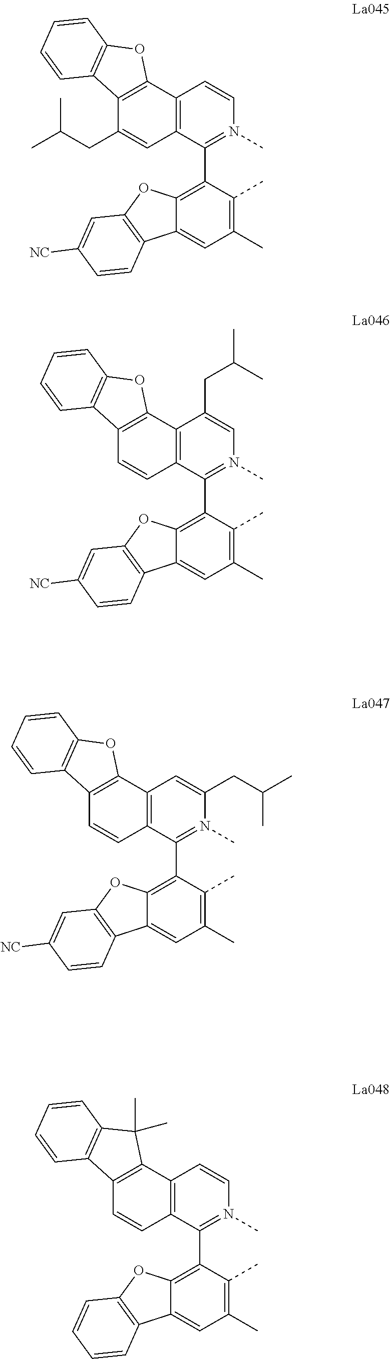

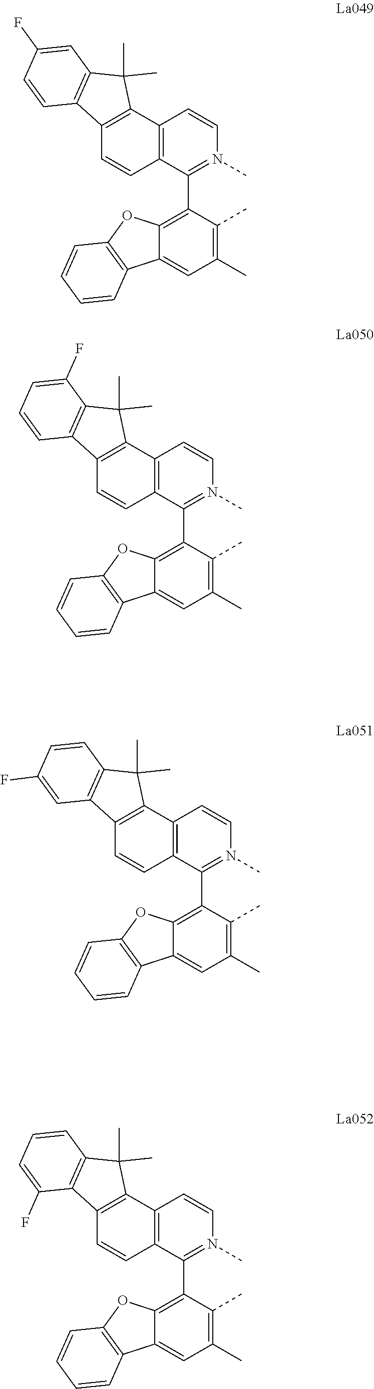

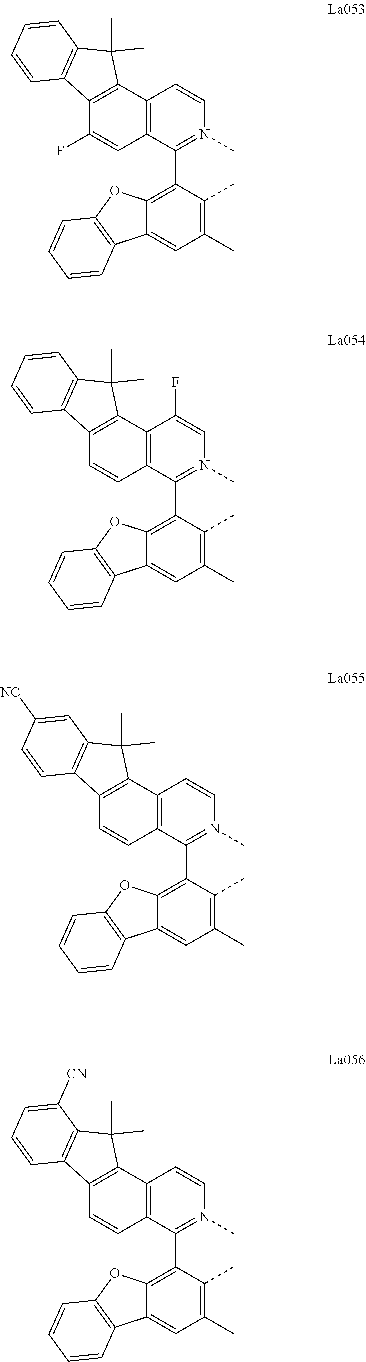

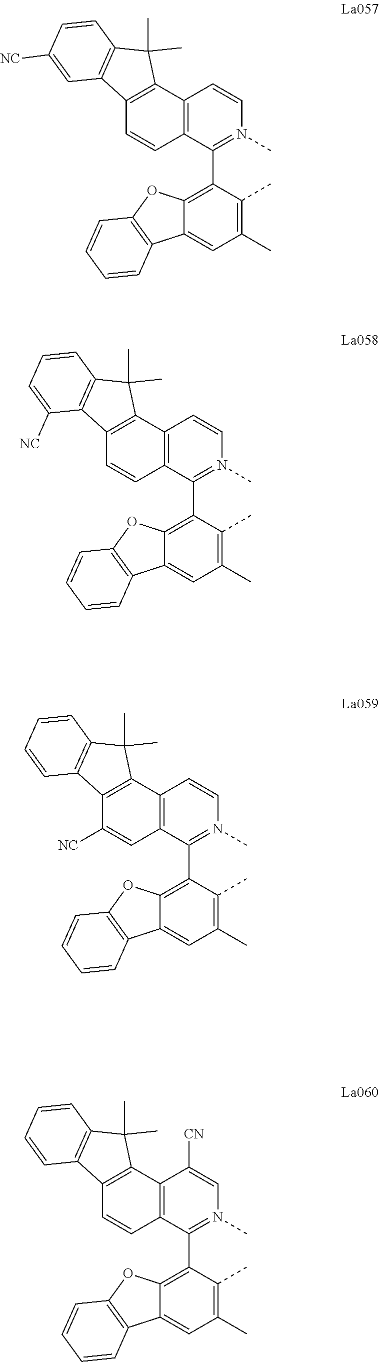

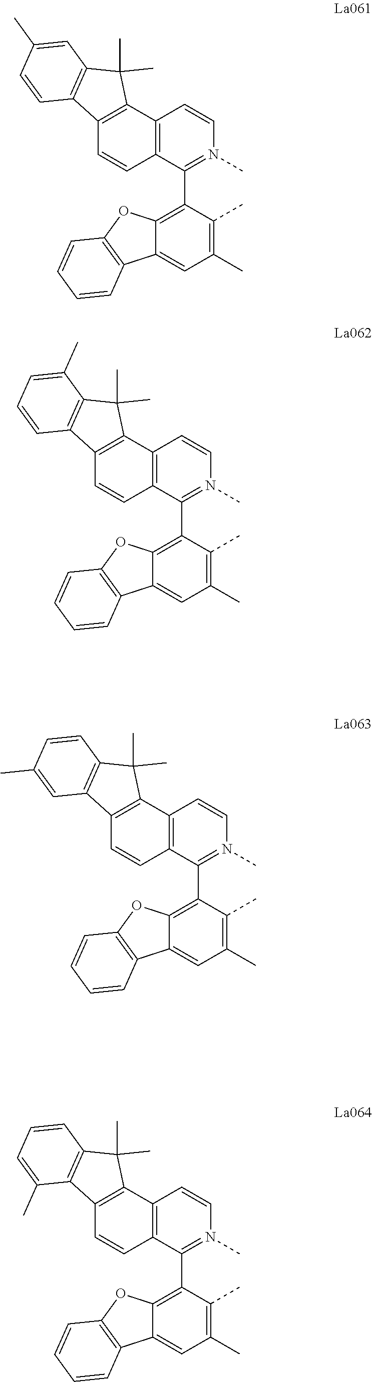

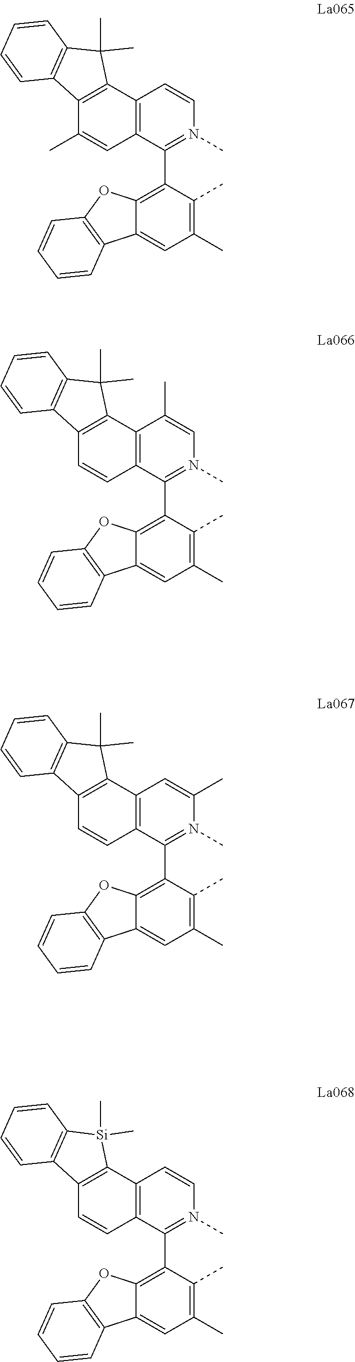

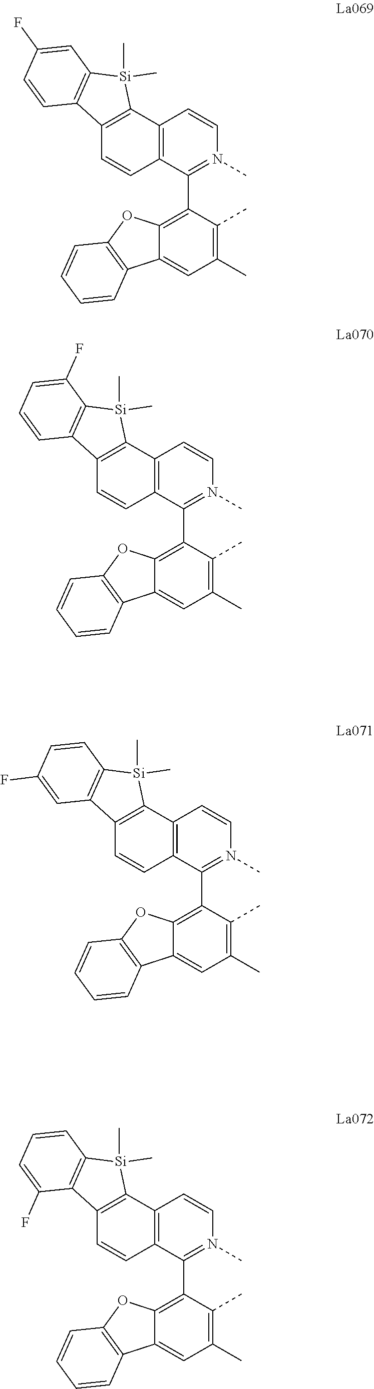

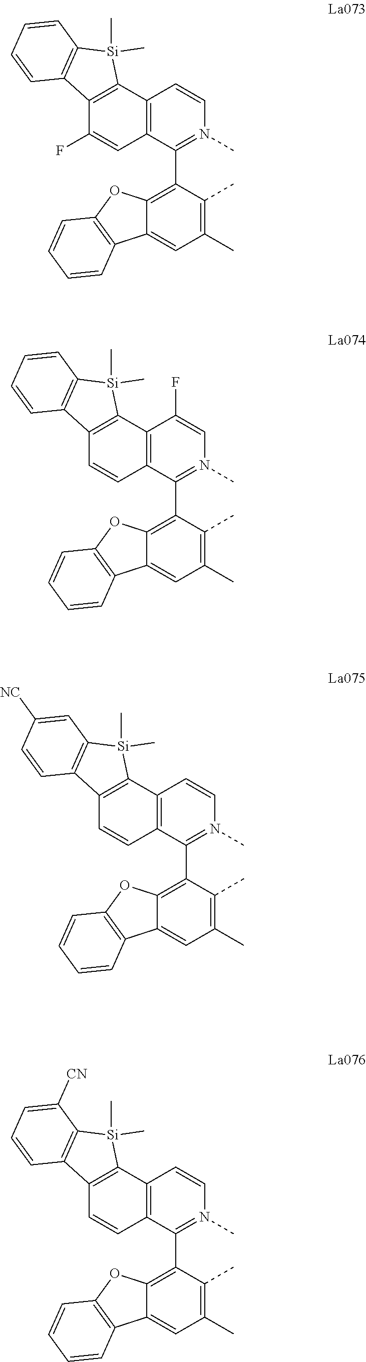

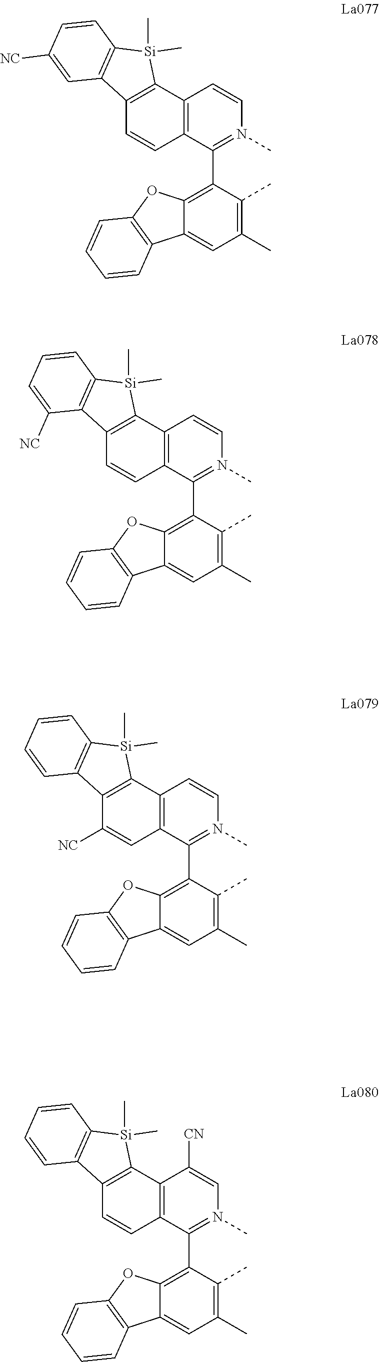

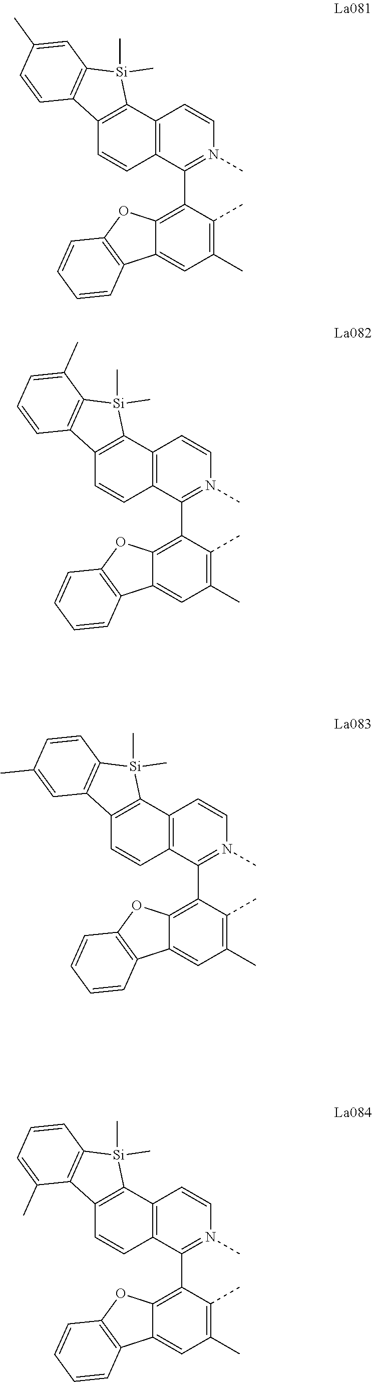

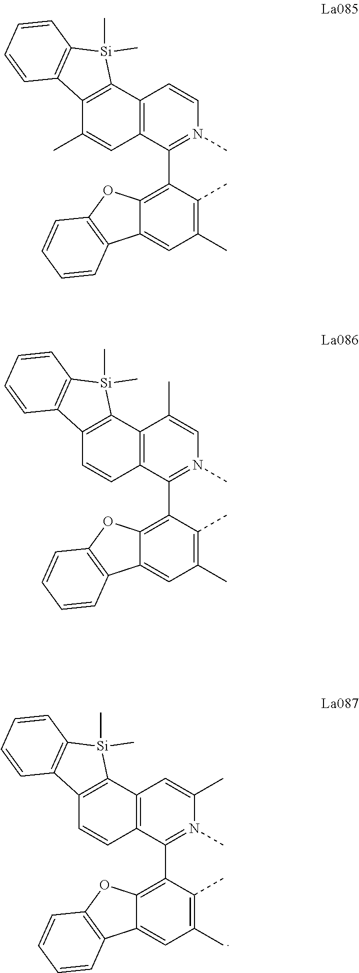

In an embodiment, Lb is one of the following structural formulas, or one of the following structural formulas in which the hydrogen atom(s) is/are partially or completely deuterated, or one of the following structural formulas in which the hydrogen atom(s) is/are partially or completely fluorinated,

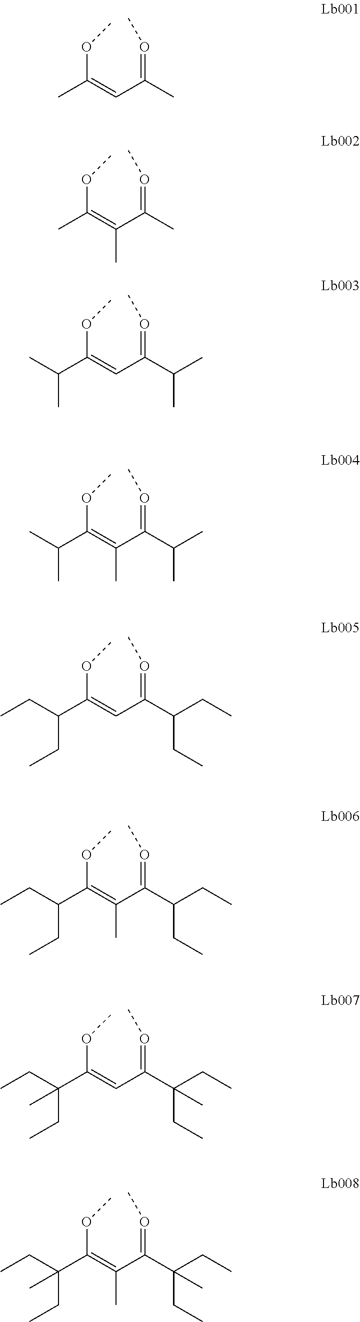

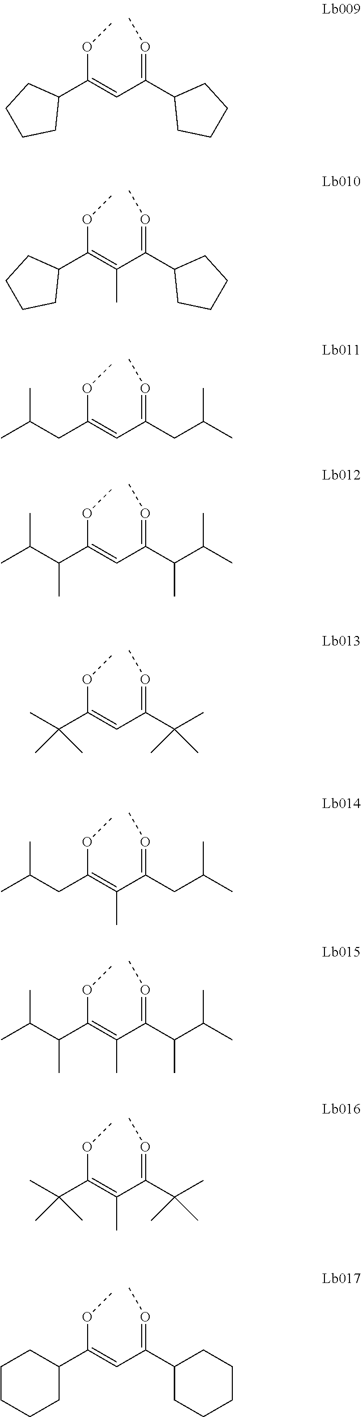

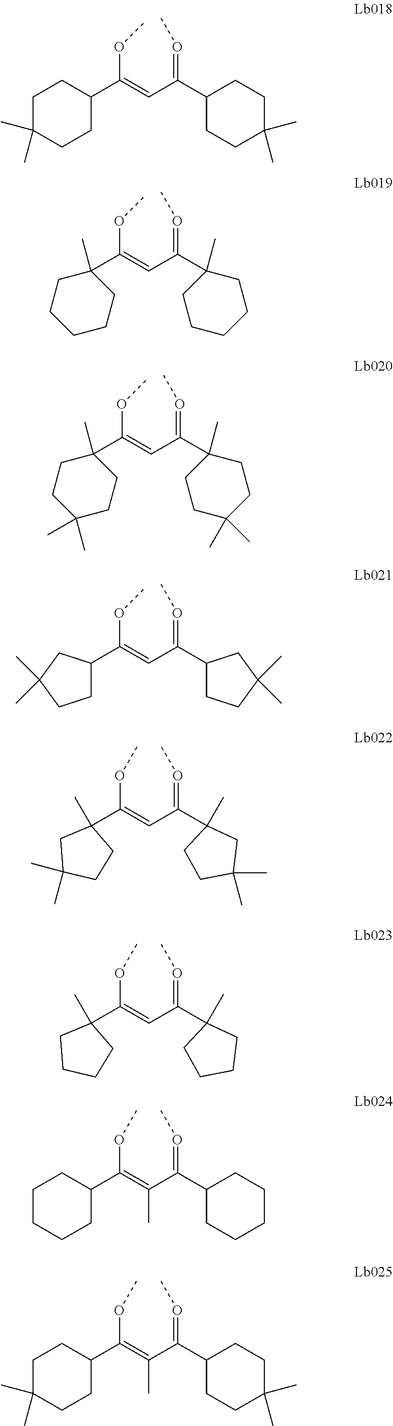

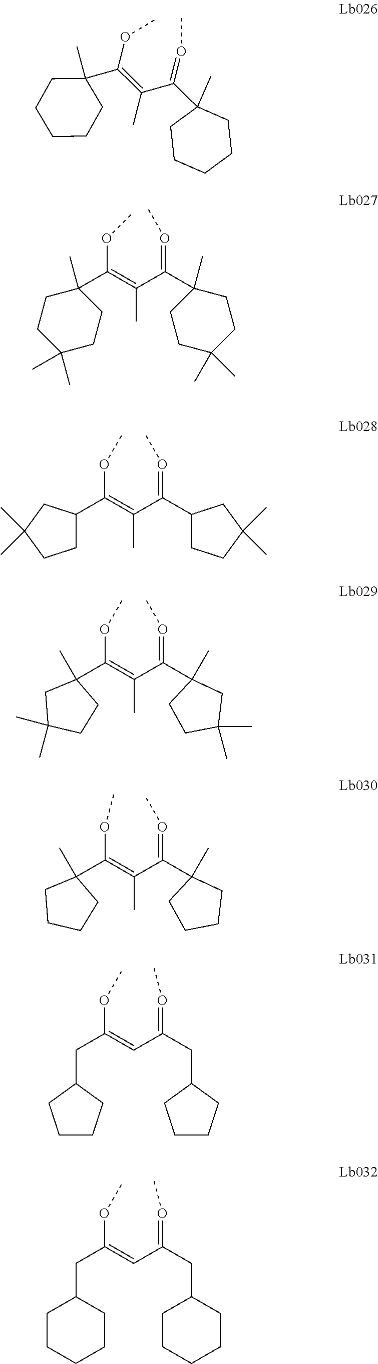

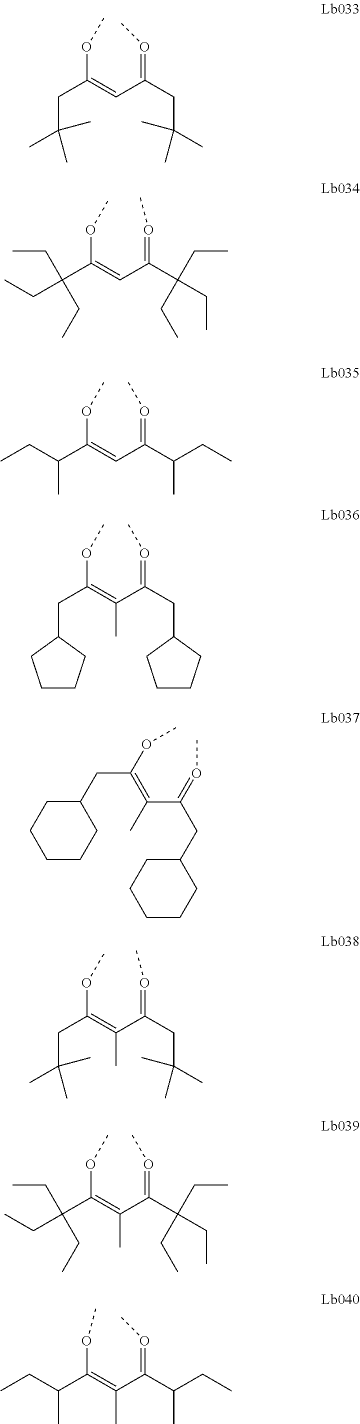

Ligand La, has a following structural formula:

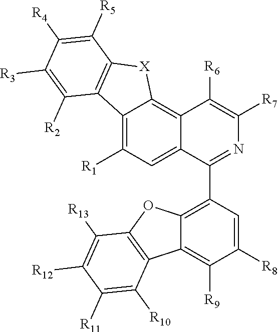

wherein R₁-R₃ are as defined above.

Furthermore, one of the objectives of the present disclosure is to provide an electroluminescent device comprising a cathode, an anode, and an organic layer arranged between the cathode and the anode, wherein the organic layer comprises the above-mentioned metal iridium complex.

The organic layer comprises a luminous layer, wherein the metal iridium complex is used as a red luminescent doping material in the luminous layer; or the organic layer comprises a hole injection layer, wherein the metal iridium complex is used as a hole injection material in the hole injection layer.

The complex material of the present disclosure has the advantages of a high optical and electrochemical stability, a high color saturation, a high luminous efficiency, a long device life, etc., which can be used in organic light-emitting devices, particularly as a red luminescent phosphorescent material, and has the potential to be applied to the AMOLED industry, particularly for display, lighting, and automobile taillights. As a phosphorescent material, the complex material of the present disclosure can convert the triplet excited state into light, so that the luminous efficiency of the organic electroluminescent device can be improved, thereby reducing the energy consumption.

### BRIEF DESCRIPTION OF DRAWINGS

FIG. 1 is a 1HNMR spectrum of Compound La001 of the present disclosure in a deuterated chloroform solution,

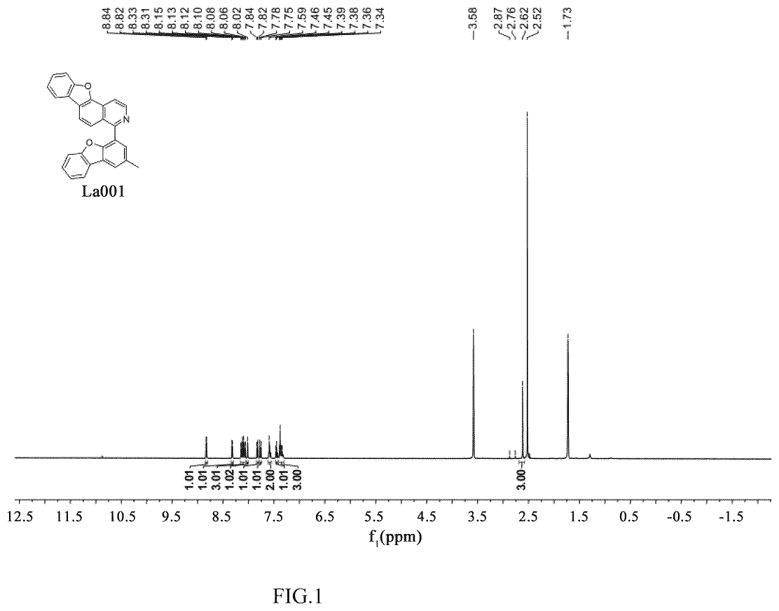

FIG. 2 is a 1HNMR spectrum of Compound Ir(La002)₂Lb005 of the present disclosure in a deuterated chloroform solution, and

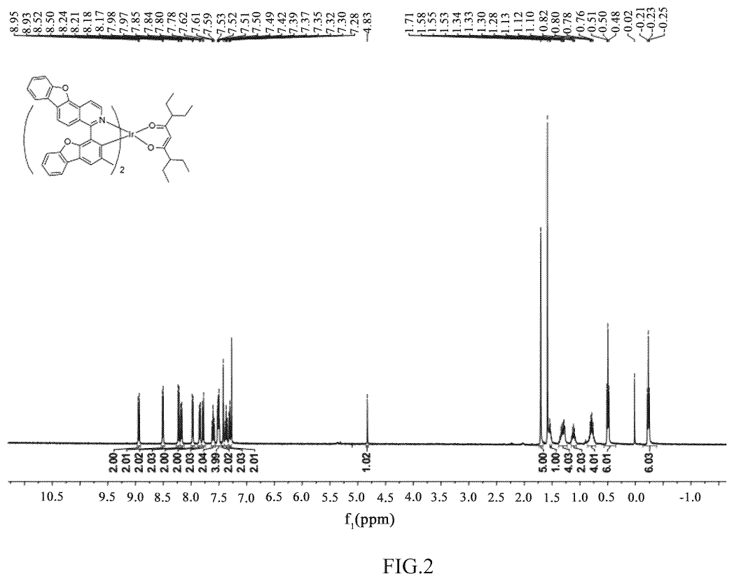

FIG. 3 is an ultraviolet absorption spectrum and emission spectrum of Compound Ir(La001)₂Lb005 in a dichloromethane solution.

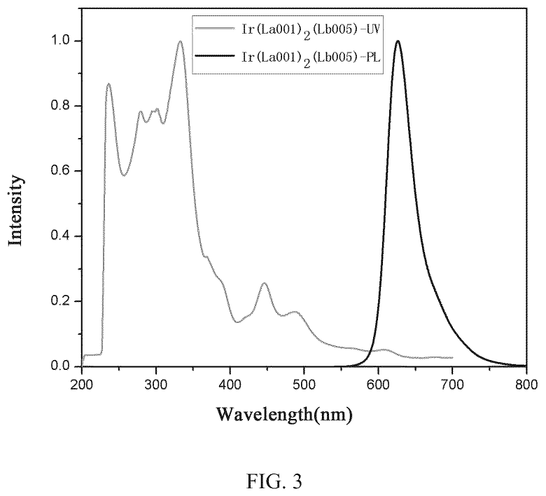

### DETAILED DESCRIPTION

The organometallic iridium compound of the present disclosure has a general formula of Ir(La)(Lb)(Lc), wherein La is a structure shown in formula (1),

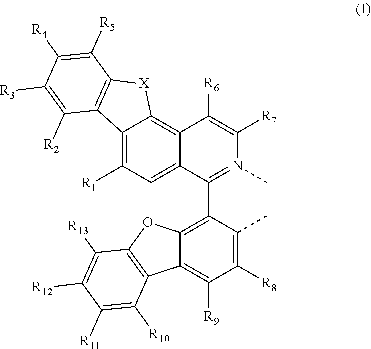

wherein a dotted line represents a position of connection to the metal Ir;
wherein X is O, S, Se, C(R₀)₂, or Si(R₀)₂;
wherein R₀-R₁₃ are each independently hydrogen, deuterium, halogen, cyano, substituted or unsubstituted C1-C10 alkyl, substituted or unsubstituted C1-C10 heteroalkyl, substituted or unsubstituted C3-C20 cycloalkyl, substituted or unsubstituted C3-C20 heterocycloalkyl, substituted or unsubstituted C2-C10 alkenyl, substituted or unsubstituted C2-C10 alkynyl, substituted or unsubstituted C6-C30 aryl, substituted or unsubstituted C2-C30 heteroaryl, substituted or unsubstituted tri(C1-C10)alkylsilyl, substituted or unsubstituted tri(C6-C12)arylsilyl, substituted or unsubstituted di(C1-C10)alkylmono(C6-C30)arylsilyl, or substituted or unsubstituted mono(C1-C10)alkyldi(C6-C30)arylsilyl, or two adjacent groups of R₁₀-R₁₃ are connected with each other to form an alicyclic ring;
wherein R₈ is not hydrogen, deuterium, halogen, or cyano;
wherein the heteroalkyl, heterocycloalkyl, and heteroaryl contain at least one O, N or S heteroatom;
wherein the substitution is substitution with deuterium, F, Cl, Br, C1-C6 alkyl, C3-C6 cycloalkyl, C1-C6 alkyl-substituted amino, cyano, isocyano or phosphine group, wherein the substitution is from mono-substitution to the maximum number of substitutions:
wherein Lb is a structure shown in formula (2),

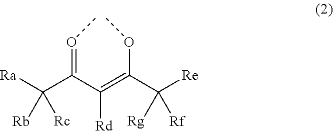

wherein a dotted line represents a position of connection to the metal Ir;
wherein Ra-Rg are each independently hydrogen, deuterium, halogen, substituted or unsubstituted C1-C10 alkyl, substituted or unsubstituted C3-C20 cycloalkyl, substituted or unsubstituted C1-C10 heteroalkyl, or substituted or unsubstituted C3-C20 heterocycloalkyl, or Ra, Rb, and Rc are connected in pairs to form an aliphatic ring, and Re, Rf, and Rg are connected in pairs to form an aliphatic ring;
wherein the heteroalkyl and heterocycloalkyl contain at least one O, N or S heteroatom;
wherein the substitution is substitution with deuterium, F, Cl, Br, C1-C4 alkyl, C1-C4 alkoxy, C3-C6 cycloalkyl, C1-C4 alkyl-substituted amino, cyano, isocyano, or phosphine group;
wherein Lc is a single anionic bidentate ligand, and Lc is different from Lb and is not an OO-type ligand;
wherein Lc and La are the same or different, the difference meaning that a mother nucleus structure is different, or the mother nucleus structure is the same but substituents are different, or the mother nucleus structure is the same and the substituents are the same but the positions of the substituents are different;
wherein La, Lb, and Lc are connected in pairs or in triplets to form a multidentate ligand.

Hereinafter, examples of each of the groups of the compound as shown in formula (1) to formula (4) will be described.

It should be noted that in the present description, the “Ca-Cb” in the expression “substituted or unsubstituted Ca-Cb X group” represents the carbon number where the X group is unsubstituted, excluding the carbon number of substituents where the X group is substituted.

In an embodiment, C1-C10 alkyl is a linear or branched alkyl group, by way of example, methyl, ethyl, propyl, isopropyl, n-butyl, isobutyl, sec-butyl, tert-butyl, n-pentyl and isomers thereof, n-hexyl and isomers thereof, n-heptyl and isomers thereof, n-octyl and isomers thereof, n-nonyl and isomers thereof, n-decyl and isomers thereof, etc., preferably methyl, ethyl, propyl, isopropyl, n-butyl, isobutyl, see-butyl, and tert-butyl, and more preferably propyl, isopropyl, isobutyl, sec-butyl and tert-butyl.

In an embodiment, C3-C20 cycloalkyl include, by way of example, cyclopropyl, cyclobutyl, cyclopentyl, cyclohexyl, 1-adamantyl, 2-adamantyl, 1-norbornyl, 2-norbornyl, etc., preferably cyclopentyl and cyclohexyl.

In an embodiment, C2-C10 alkenyl include, by way of example, vinyl, propenyl, allyl, 1-butadienyl, 2-butadienyl, 1-hexatrienyl, 2-hexatrienyl, 3-hexatrienyl, etc., preferably propenyl and allyl.

In an embodiment, C1-C10 heteroalkyl is linear or branched alkyl, cycloalkyl, etc., which contains atoms other than carbon and hydrogen, and include, by way of example, mercaptomethylmethanyl, methoxymethanyl, ethoxymethanyl, tert-butoxymethanyl, N,N-dimethylmethanyl, epoxybutanyl, epoxypentanyl, epoxyhexanyl, etc., preferably methoxymethanyl and epoxypentanyl.

In an embodiment, specific examples of aryl are phenyl, naphthalenyl, anthracenyl, phenanthrenyl, tetraphenyl, pyrenyl, chrysenyl, benzo[c]phenanthrenyl, benzo[g]chrysenyl, fluorenyl, benzofluorenyl, dibenzofluorenyl, biphenyl, terphenyl, quaterphenyl, fluoranthenyl, etc., preferably phenyl and naphthalenyl.

In an embodiment, specific examples of heteroaryl include, by way of example, pyrrolyl, pyrazinyl, pyridyl, pyrimidinyl, triazinyl, indolyl, isoindolyl, imidazolyl, furyl, benzofuranyl, isobenzofuranyl, dibenzofuranyl, dibenzothienyl, azadibenzofuranyl, azadibenzothienyl, diazadibenzofuranyl, diazadibenzothienyl, quinolyl, isoquinolyl, quinoxalinyl, carbazolyl, phenanthridinyl, acridinyl, phenanthrolinyl, phenazinyl, phenothiazinyl, phenoxazinyl, oxazolinyl, oxadiazolyl, furazanyl, thienyl, benzothienyl, dihydroacridinyl, azacarbazolyl, diazacarbazolyl, quinazolinyl, etc., preferably pyridyl, pyrimidinyl, triazinyl, dibenzofuranyl, dibenzothienyl, azadibenzofuranyl, azadibenzothienyl, diazadibenzofuranyl, diazadibenzothienyl, carbazolyl, azacarbazolyl, and diazacarbazolyl.

The following examples are only for the convenience of understanding the present disclosure and should not be regarded as specific limitations to the present disclosure.

Raw materials, solvents, etc., involved during the synthesis of compounds in the present disclosure are all purchased from suppliers familiar to those skilled in the art, such as Alfa and Acros.

### Synthesis of Compound La001

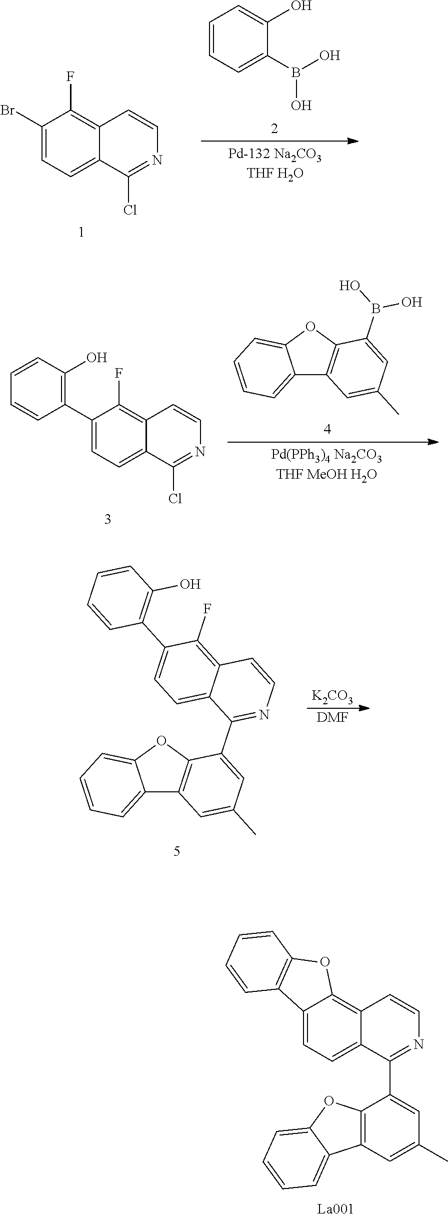

### Synthesis of Compound 3

Compound 1 (13.00 g, 49.9 mmol, 1.0 eq), Compound 2 (7.23 g, 52.4 mmol 1.05 eq), dichloro-di-tert-butyl-(4-dimethylaminophenyl)phosphinepalladium(II) (176.7 mg, 0.25 mmol, 0.05 eq), sodium carbonate (10.58 g, 99.8 mmol, 2.00 eq), tetrahydrofuran (195 mL), and deionized water (65 mL) were added to a 500 mL three-necked flask, and the flask was evacuated and displaced with nitrogen 3 times. Under nitrogen protection, the temperature was raised to 50° C. and the reaction was conducted under stirring for 4 hours. TLC monitoring showed that Compound 1 was completely reacted. After cooling to room temperature, the reaction solution was left to stand for liquid separation, and the organic phase was collected, dried by rotary evaporation, and then separated by column chromatography (the eluent was tetrahydrofuran:n-hexane=1:10). After concentration, Compound 3 was obtained as a white solid (9.5 g, yield: 69.54%). Mass spectrum: 274.69 (M+H).

### Synthesis of Compound 5

Compound 3 (9.5 g, 34.7 mmol, 1.0 eq), Compound 4 (8.63 g, 38.2 mmol, 1.05 eq), tetrakis(triphenylphosphine)palladium (2.0 g, 1.73 mmol, 0.05 eq), sodium carbonate (7.36 g, 69.4 mmol, 2.00 eq), tetrahydrofuran (142.5 mL), methanol (47.5 mL), and deionized water (47.5 ml) were added to a 500 mL three-necked flask, and the flask was evacuated and displaced with nitrogen 3 times. Under nitrogen protection, the temperature was raised to 50° C. and the reaction was conducted under stirring for 4.5 hours. TLC monitoring showed that Compound 3 was completely reacted. After cooling to room temperature, the reaction solution was left to stand for liquid separation, and the aqueous phase was extracted with ethyl acetate (100 mL*3). The collected organic phases were combined, dried by rotary evaporation, and then separated by column chromatography (the eluent was tetrahydrofuran:n-hexane=: 4). After concentration, Compound 5 was obtained as a white solid (1311 g, yield: 90.03%). Mass spectrum: 420.45 (M+H).

### Synthesis of Compound La001

Compound 5 (13.1 g, 31.2 mmol, 1.0 eq), potassium carbonate (12.93 g, 93.7 mmol, 3.0 eq), N,N-dimethylformamide (524 mL) were added to a 1 L three-necked flask, and the flask was evacuated and displaced with nitrogen 3 times. Under nitrogen protection, the temperature was raised to 110° C. and the reaction was conducted under stirring for 16 hours. TLC monitoring showed that Compound 5 was completely reacted. After cooling to room temperature, the reaction solution was slowly added to deionized water (2.5 L), stirred for 1 h, and filtered to obtain a solid. The solid was added to N,N-dimethylformamide for recrystallization twice (product:N,N-dimethylformamide=1:5), and after drying, Compound La001 was obtained as a white solid (8.6 g, yield: 99.91%). Mass spectrum: 400.44 (M+H). ¹HNMR (400 MHz, D8-THF) δ 8.83 (d, J=5.6 Hz, 1H), 8.32 (d, J=5.7 Hz, 1H), 8.16-8.05 (m, 3H), 8.02 (s, 11H), 7.83 (d, J=8.3 Hz, 1H), 777 (d, J=8.8 Hz, 114), 759 (s, 2H), 7.46 (d, J 7.2 Hz, 1H), 7.37 (dd, J=15.3, 6.6 Hz, 3H), 2.62 (s, 3H).

### Synthesis of Compound Ir(La001)₂(Lb005)

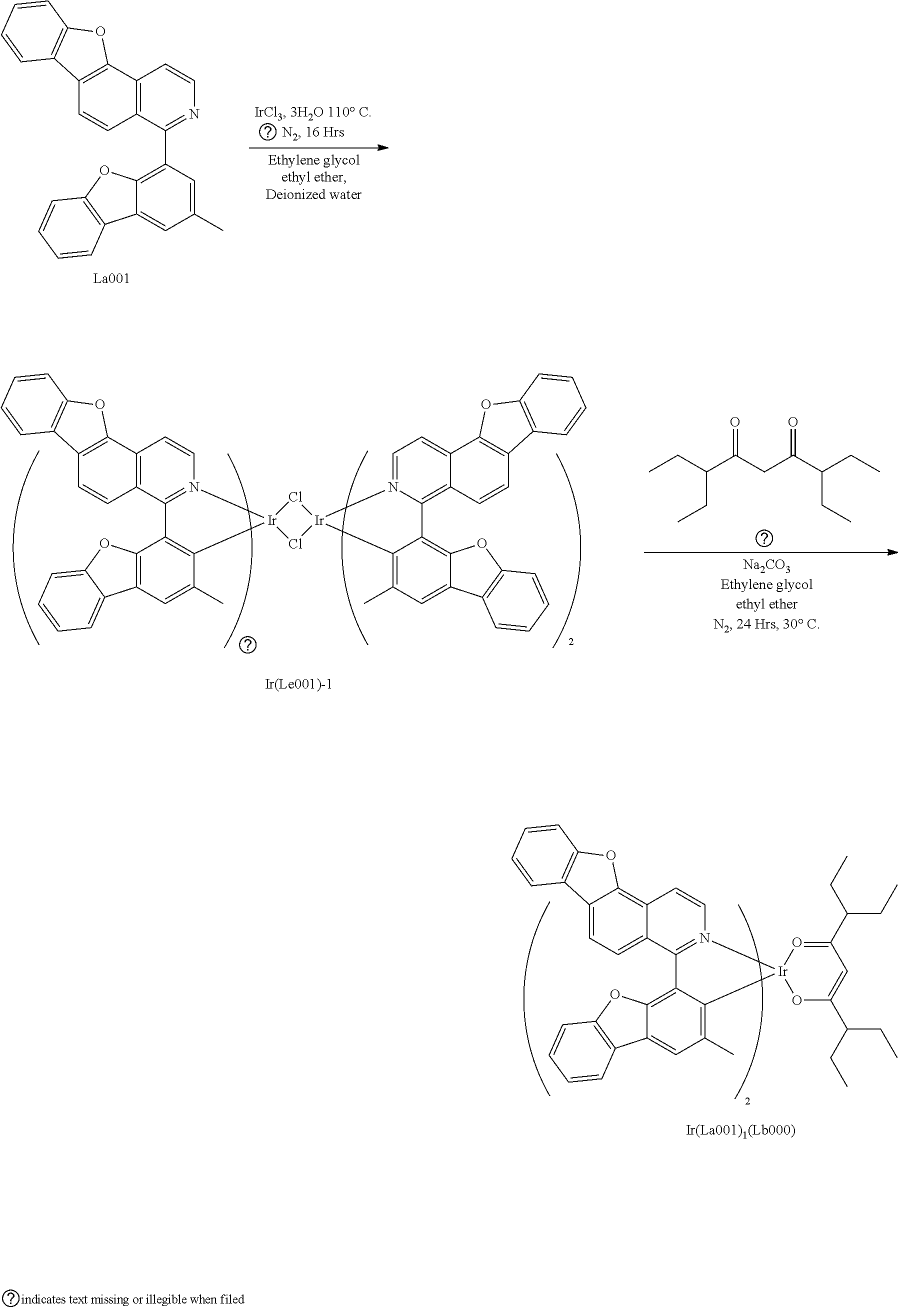

### Synthesis of Compound Ir(La001)-1

Compound La001 (8.0 g, 20.03 mmol, 3.5 eq) and IrCl₃·3H₂O (2.02 g, 5.72 mmol, 1.0 eq) were placed in a 500 mL single-necked round-bottom flask, added with tetrahydrofuran (240 mL) and deionized water (24 mL), and the flask was evacuated and displaced with nitrogen 3 times. The resulting mixed solution was stirred at 80° C. for 48 hours under N₂ protection. After cooling to room temperature, methanol (250 mL) was added and stirred to precipitate out a solid, and the solid was collected by filtration and dried to obtain Compound Ir(La001)-1 as a dark red oil (5.48 g, 93.54%). The obtained compound could be directly used in the next step without further purification.

### Synthesis of Compound Ir(La001)₂(Lb005)

Compound Ir(La001)-1 (5.48 g, 5.35 mmol, 1.0 eq), Lb005 (5.68 g, 26.74 mmol, 5.0 eq), and sodium carbonate (5.67 g, 53.49 mmol, 10.0 eq) were placed in a 500 mL single-necked round-bottom flask, added with tetrahydrofuran (180 mL), and the flask was evacuated and displaced with nitrogen 3 times. The resulting mixed solution was stirred at 60° C. for reaction for 48 hours under N₂ protection. TLC monitoring showed that Ir(La001)-1 was completely reacted. After cooling to room temperature, the reaction solution was added with 180 mL of methanol for pulping at room temperature for 1 h, and subjected to suction filtration. The filter cake was dissolved with dichloromethane (40 mL) and then filtered with silica gel, and the filtrate was washed 3 times by adding deionized water (20 mL). After liquid separation, the organic phase was collected, concentrated and dried to obtain a dark red solid, which was recrystallized twice with tetrahydrofuran/methanol (product/tetrahydrofuran/methanol=1 g/9 mL/9 mL) and dried to obtain Compound Ir(La001)₂(Lb005) as a red solid (3.87 g, yield: 60.26%). 3.87 g of crude Ir(La001)₂(Lb005) was sublimated and purified to obtain sublimated pure Ir(La001)₂(Lb005) (1.96 g, yield: 50.64%). Mass spectrum: 1201.40 (M+H). HNMR (400 MHz, CDCl₃) δ 8.94 (d, J=9.0 Hz, 2H), 8.51 (d, J=6.4 Hz, 2H), 8.22 (d, J=8.9 Hz, 2H), 8.18 (d, J=7.4 Hz, 2H), 7.97 (d, J=: 6.2 Hz, 2H), 7.84 (d, J=7.1 Hz, 2H), 7.79 (d, J=8.2 Hz, 2H), 7.61 (t, J=7.2 Hz, 2H), 7.56-7.45 (m, 4H), 7.42 (s, 2H), 7.37 (t, J=7.8 Hz, 2H), 7.30 (t, J=7.0 Hz, 2H), 4.83 (s, 1H), 1.71 (s, 5H), 1.53 (s, 1H), 1.31 (dd, J=15.4, 7.0 Hz, 4H), 1.16-1.06 (m, 2H), 0.79 (dd, J=14.4, 6.7 Hz, 41H), 0.50 (t, J=7.4 Hz, 6H), −0.23 (t, J 7.4 Hz, 6H).

### Synthesis of Compound La005

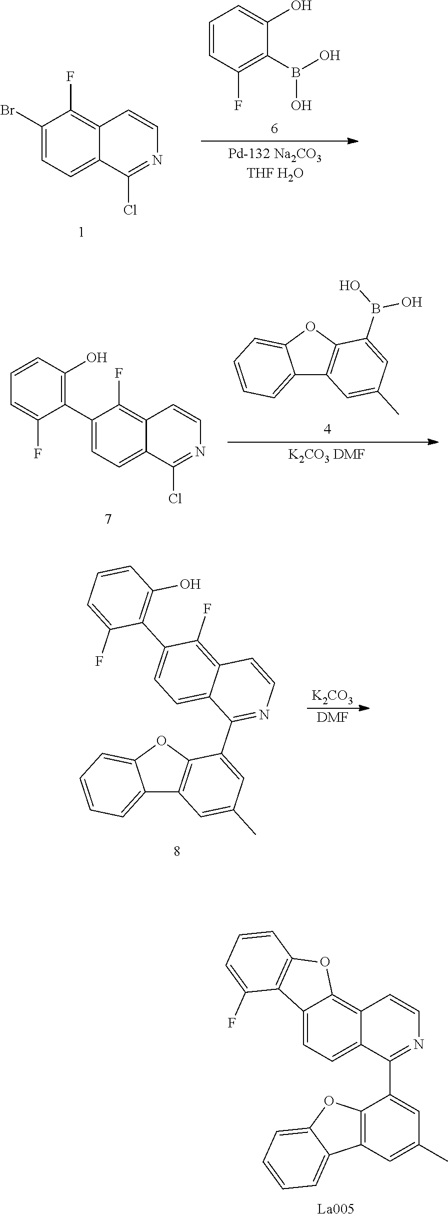

### Synthesis of Compound 7

Referring to the synthesis and purification method for Compound 3, only by changing the corresponding raw materials, target Compound 7 was obtained. Mass spectrum: 292.68 (M+H).

### Synthesis of Compound 8

Referring to the synthesis and purification method for Compound 5, only by changing the corresponding raw materials, target Compound 8 was obtained. Mass spectrum: 438.44 (M+H).

### Synthesis of Compound La005

Referring to the synthesis and purification method for Compound La001, only by changing the corresponding raw materials, target Compound La005 was obtained. Mass spectrum: 418.43 (M+H).

### Synthesis of Compound Ir(La005)₂(Lb005)

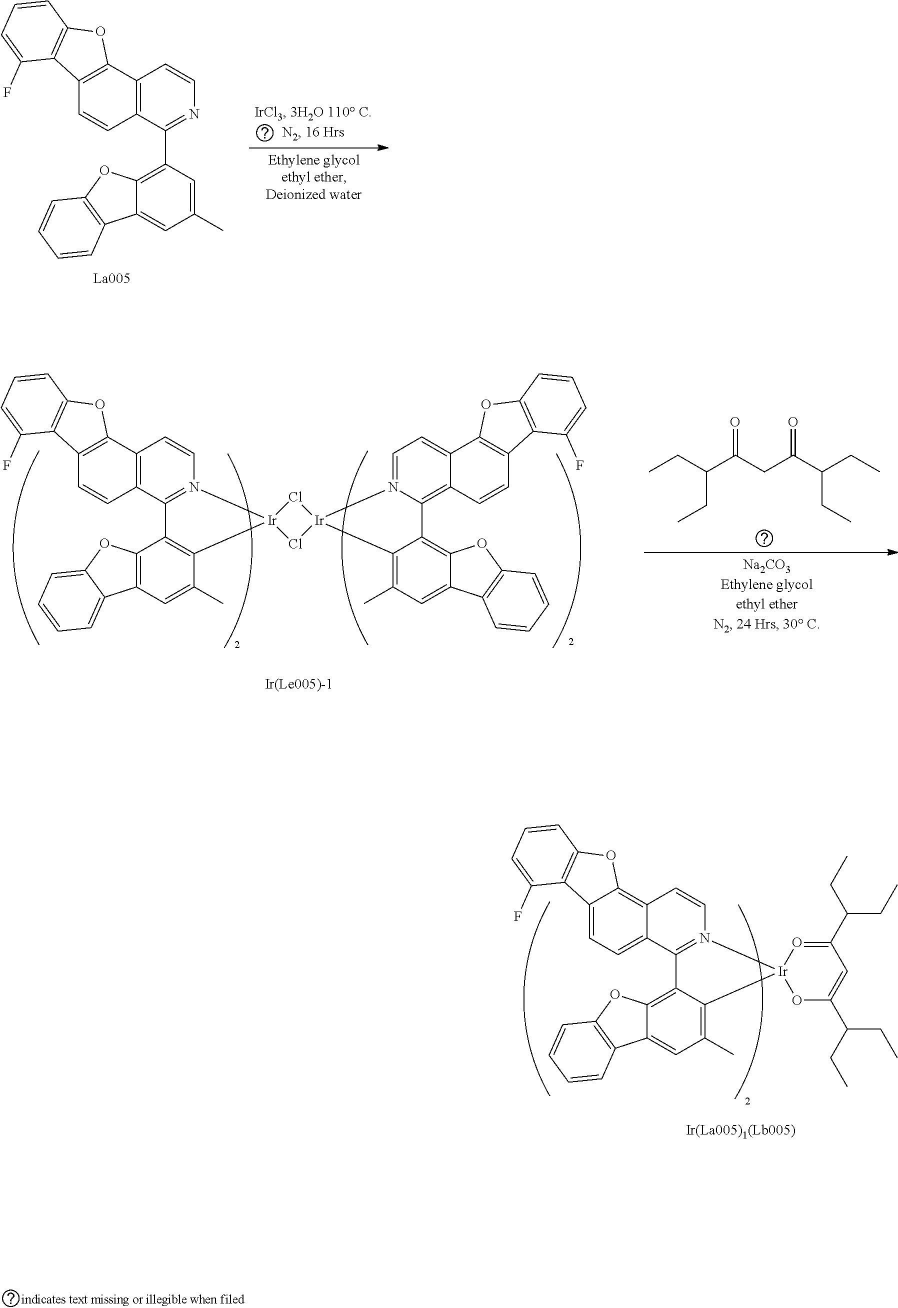

### Synthesis of Compound Ir(La005)-1

Referring to the synthesis and purification method for Compound Ir(La001)-1, by changing the corresponding raw materials, Compound Ir(La005)-1 was obtained which was directly used in the next step without purification.

### Synthesis of Compound Ir(La005)₂(Lb005)

Referring to the synthesis and purification method for Compound Ir(La001)₂(Lb005), only by changing the corresponding raw materials, Compound Ir(La005)₂(Lb005) was obtained as a red solid (3.21 g, yield: 46.77%). 3.21 g of crude Ir(La005)₂(Lb005) was sublimated and purified to obtain sublimated pure Ir(La005)₂(Lb005) (1.89 g, yield: 58.87%). Mass spectrum: 1237.38 (M+H). ¹H NMR (400 MHz, CDCl₃) δ 8.92 (d, J=8.7 Hz, 2H), 8.49 (d, J=6.5 Hz, 2H), 8.21 (d, J=7.7 Hz, 2H), 7.94 (d, J=6.4 Hz, 2H), 7.81 (d, J=7.1 Hz, 2H), 7.72 (d, J=8.4 Hz, 2H), 7.60 (t, J=7.6 Hz, 2H), 7.54-7.42 (m, 4H), 7.38 (s, 2H), 7.34 (t, J=7.6 Hz, 2H), 7.28 (t, J=7.2 Hz, 2H), 4.81 (s, 1H), 1.69 (s, 5H), 1.52 (s, 1H), 1.32 (dd, J=15.4, 7.0 Hz, 4H), 1.16-1.06 (m, 2H), 0.82 (dd, J=14.4, 6.7 Hz, 4H), 0.61 (t, J=7.4 Hz, 6H), −0.18 (t, J=7.4 Hz, 6H).

### Synthesis of Compound La007

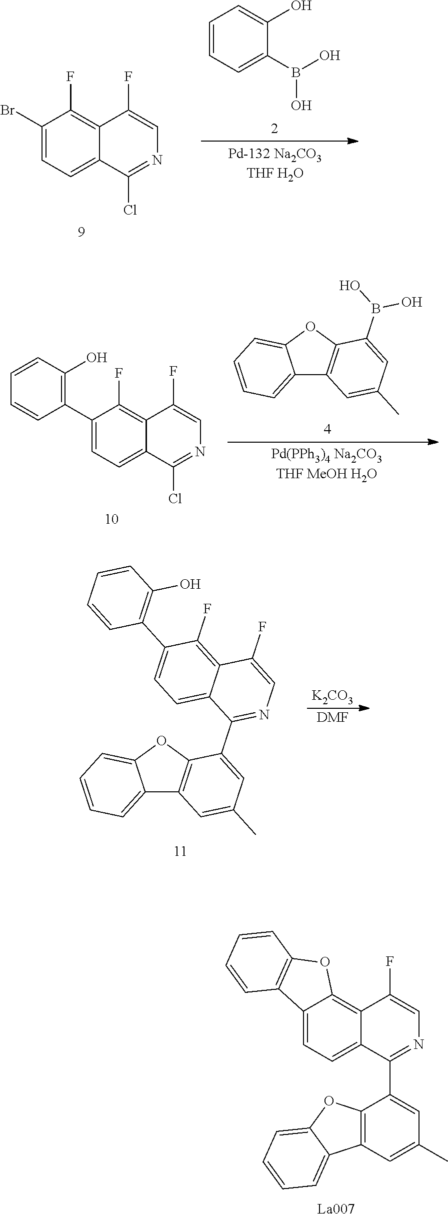

### Synthesis of Compound 10

Referring to the synthesis and purification method for Compound 3, only by changing the corresponding raw materials, target Compound 10 was obtained. Mass spectrum: 292.68 (M+H).

### Synthesis of Compound 11

Referring to the synthesis and purification method for Compound 5, only by changing the corresponding raw materials, target Compound 11 was obtained. Mass spectrum: 438.44 (M+H).

### Synthesis of Compound La007

Referring to the synthesis and purification method for Compound La001, only by changing the corresponding raw materials, target Compound La007 was obtained. Mass spectrum: 418.43 (M+H).

### Synthesis of Compound Ir(La007)₂(Lb005)

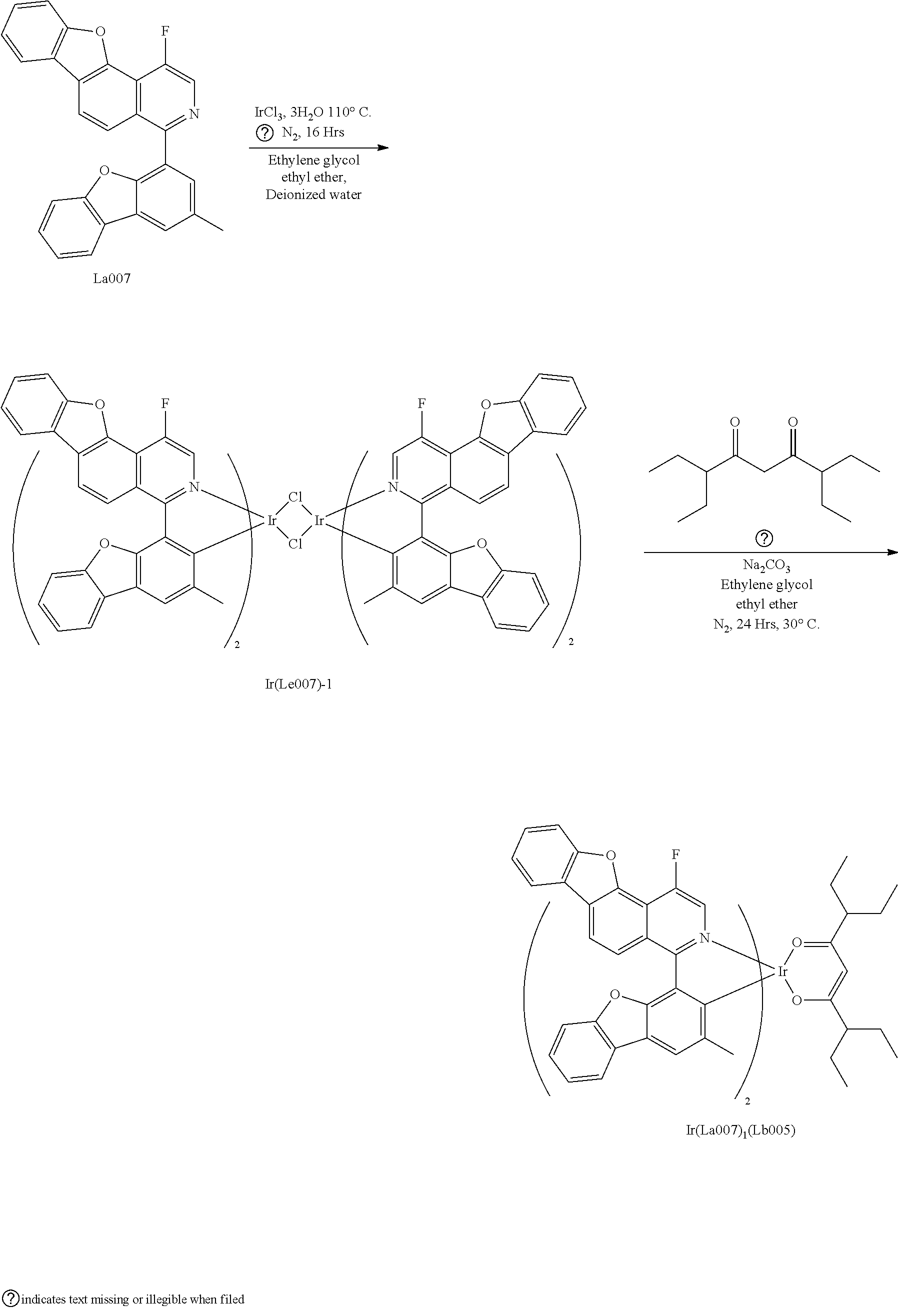

### Synthesis of Compound Ir(La007)-1

Referring to the synthesis and purification method for Compound Ir(La001)-1, by changing the corresponding raw materials, Compound Ir(La007)-1 was obtained which was directly used in the next step without purification.

### Synthesis of Compound Ir(La007)₂(Lb005)

Referring to the synthesis and purification method for Compound Ir(La001)₂(Lb005), only by changing the corresponding raw materials, Compound Ir(La007)₂(Lb005) was obtained as a red solid (3.17 g, yield: 44.92%). 3.17 g of crude Ir(La007)₂(Lb005) was sublimated and purified to obtain sublimated pure Ir(La007)₂(Lb005) (1.78 g, yield: 56.15%). Mass spectrum: 1237.38 (M+H). ¹H NMR (400 MHz, CDCl₃) δ 8.95 (s, 2H), 8.46 (d, J=6.1 Hz, 2H), 8.25 (d, J=7.9 Hz, 2H), 8.21 (d, J=7.6 Hz, 2H), 7.83 (d, J=7.1 Hz, 2H), 7.77 (d, J 8.2 Hz, 2H), 7.62 (t, J=7.2 Hz, 2H), 7.51-7.42 (m, 4H), 7.39 (s, 2H), 7.35 (t, J=7.8 Hz, 2H), 7.31 (t, J=7.0 Hz, 2H), 4.82 (s, 1H), 1.72 (s, 5H), 1.54 (s, 1H), 1.26 (dd, J=15.4, 7.0 Hz, 4H), 1.18-1.09 (m, 2H), 0.82 (dd, J=14.4, 6.7 Hz, 4H), 0.52 (t, J=7.4 Hz, 6H), −0.19 (t, J 7.4 Hz, 6H).

### Synthesis of Compound La011

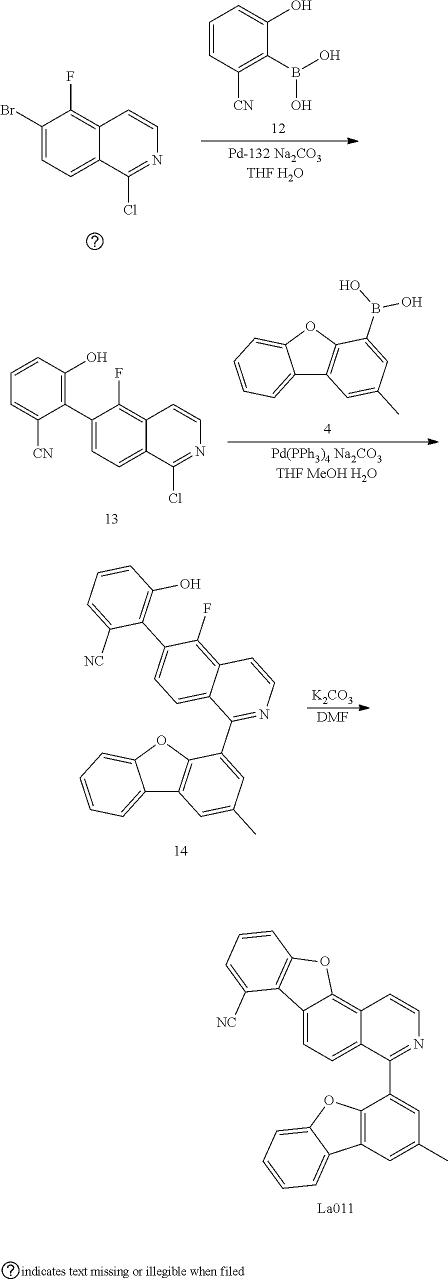

### Synthesis of Compound 13

Referring to the synthesis and Purification method for Compound 3, only by changing the corresponding raw materials, target Compound 13 was obtained. Mass spectrum: 299.7 (M+H).

### Synthesis of Compound 14

Referring to the synthesis and purification method for Compound 5, only by changing the corresponding raw materials, target Compound 14 was obtained. Mass spectrum: 445.46 (M+H).

### Synthesis of Compound La011

Referring to the synthesis and purification method for Compound La001, only by changing the corresponding raw materials, target Compound La011 was obtained. Mass spectrum: 425.45 (M+H).

### Synthesis of Compound Ir(La011)₂(Lb005)

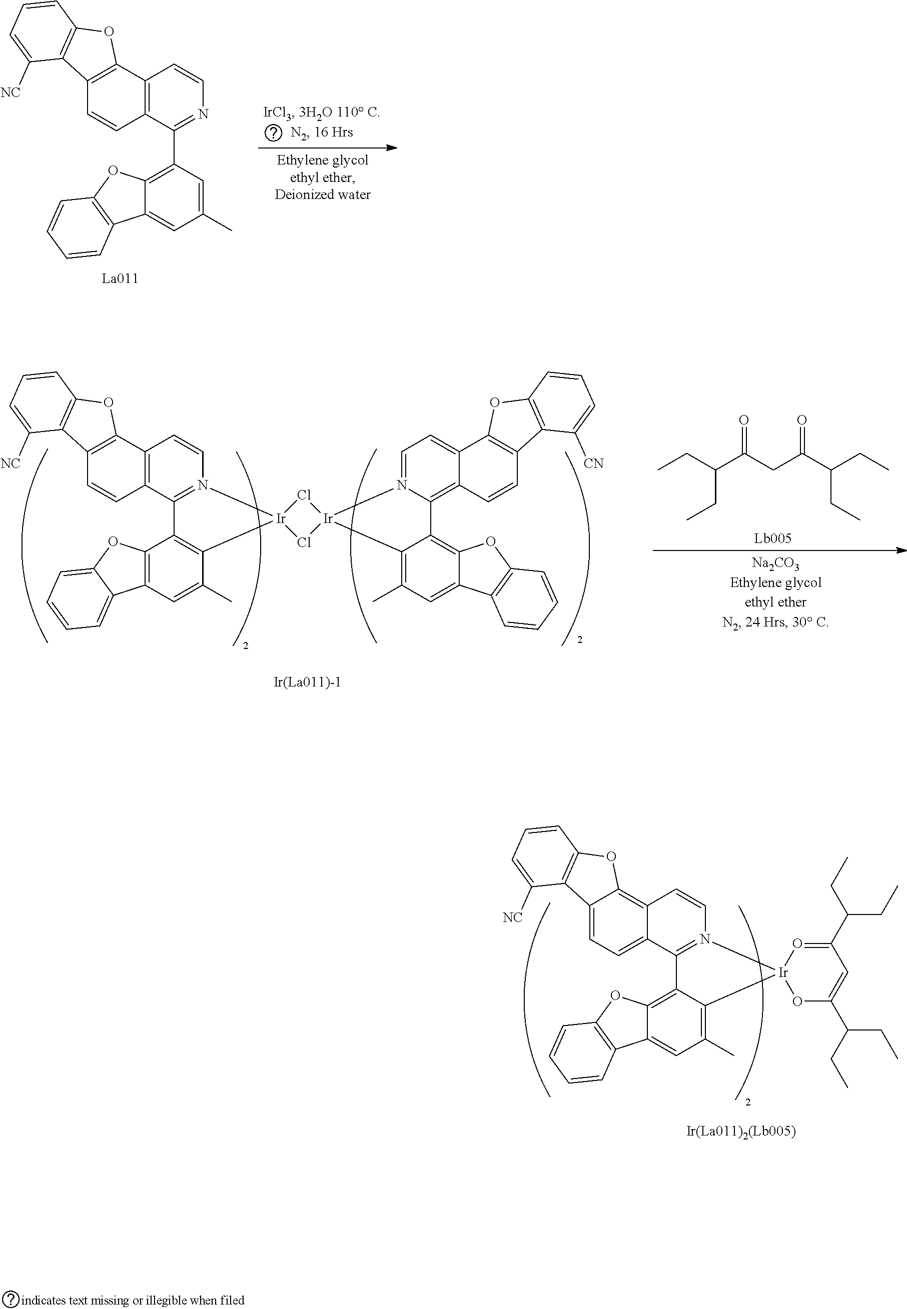

### Synthesis of Compound Ir(La011)-1

Referring to the synthesis and purification method for Compound Ir(La001)-1, by changing the corresponding raw materials, Compound Ir(La011)-1 was obtained which was directly used in the next step without purification.

### Synthesis of Compound Ir(La011)₂(Lb005)

Referring to the synthesis and purification method for Compound Ir(La001)₂(Lb005), only by changing the corresponding raw materials, Compound Ir(La011)₂(Lb005) was obtained as a red solid (2.86 g, yield: 45.67%). 2.86 g of crude Ir(La011)₂(Lb005) was sublimated and purified to obtain sublimated pure Ir(La011)₂(Lb005) (1.69 g, yield: 59.09%). Mass spectrum: 1251.42 (M+H). ¹H NMR (400 MHz, CDCl₃) δ 8.93 (d, J=9.1 Hz, 2H), 8.51 (d, J=6.3 Hz, 2H), 8.22 (d, J=7.3 Hz, 2H), 7.96 (d, J=6.4 Hz, 2H), 7.83 (d, J=7.2 Hz, 2H), 7.74 (d, J=7.8 Hz, 2H), 7.62 (t, J=6.8 Hz, 2H), 7.55-7.44 (m, 4H), 7.37 (s, 2H), 7.35 (t, J=7.8 Hz, 2H), 7.27 (t, J=7.0 Hz, 2H), 4.83 (s, 1H), 1.71 (s, 5H), 1.55 (s, 1H), 1.34 (dd, J=15.4, 7.0 Hz, 4H), 1.17-1.07 (m, 2H), 0.84 (dd, J=14.4, 6.7 Hz, 4H), 0.63 (t, J=7.4 Hz, 6H), −0.24 (t, J=7.4 Hz, 6H).

### Synthesis of Compound La014

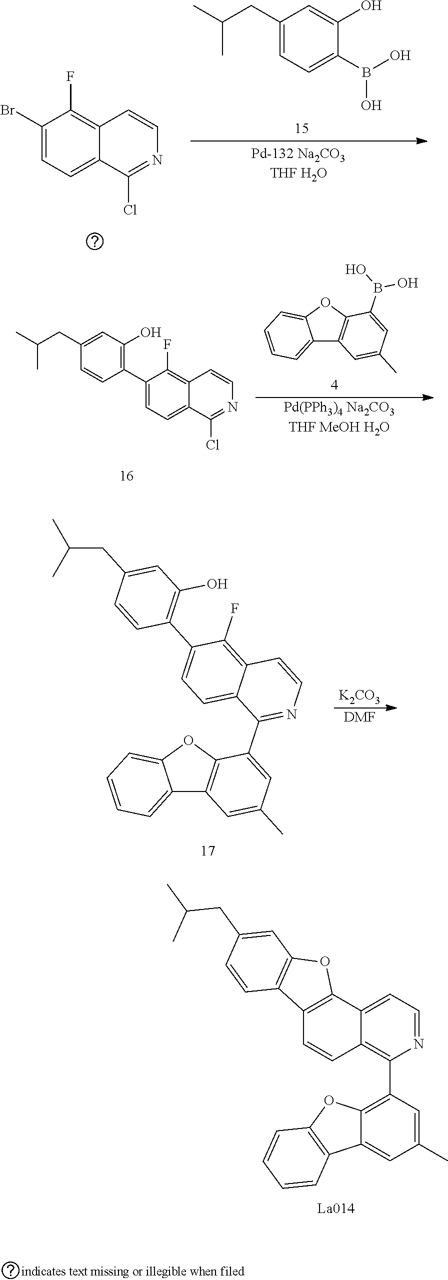

### Synthesis of Compound 16

Referring to the synthesis and purification method for Compound 3, only by changing the corresponding raw materials, target Compound 16 was obtained. Mass spectrum 330.8 (M+1H).

### Synthesis of Compound 17

Referring to the synthesis and purification method for Compound 5, only by changing the corresponding raw materials, target Compound 17 was obtained. Mass spectrum: 476.55 (M+H).

### Synthesis of Compound La014

Referring to the synthesis and purification method for Compound La001, only by changing the corresponding raw materials, target Compound La014 was obtained. Mass spectrum: 456.55 (M+H).

### Synthesis of Compound Ir(La014)₂(Lb005)

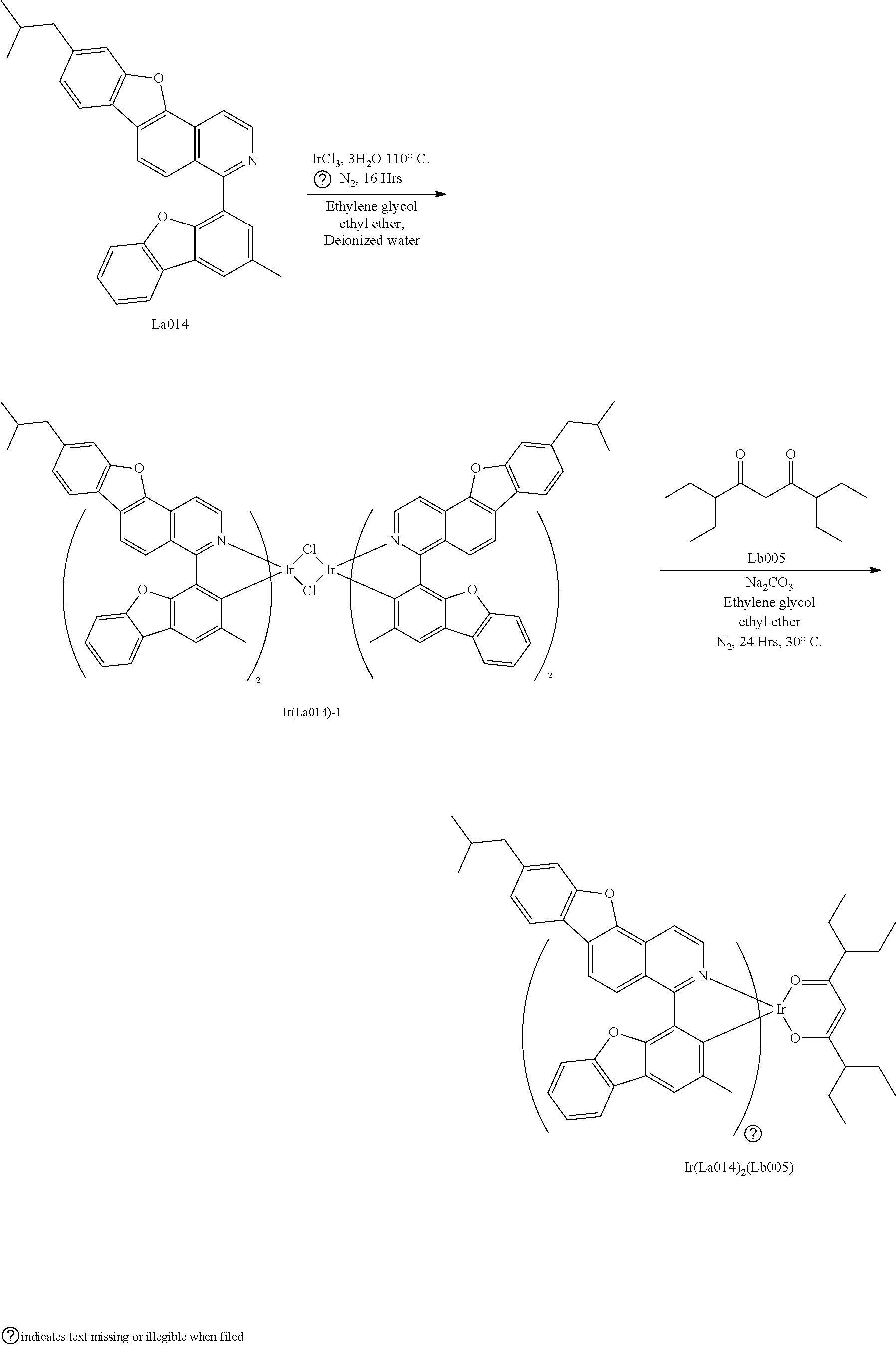

### Synthesis of Compound Ir(La014)-1

Referring to the synthesis and purification method for Compound Ir(La001)-1, by changing the corresponding raw materials, Compound Ir(La014)-1 was obtained which was directly used in the next step without purification.

### Synthesis of Compound Ir(La014)₂(Lb005)

Referring to the synthesis and purification method for Compound Ir(La001)₂(Lb005), only by changing the corresponding raw materials, Compound Ir(La014)₂(Lb005) was obtained as a red solid (3.05 g, yield: 48.61%). 3.05 g of crude Ir(La014)₂(Lb005) was sublimated and purified to obtain sublimated pure Ir(La014)₂(Lb005) (1.92 g, yield: 62.95%) Mass spectrum: 1313.61 (M+H). ¹H NMR (400 MHz, CDCl₃) δ 8.90 (d, J=9.2 Hz, 2H), 8.49 (d, J=6.3 Hz, 2H), 8.21 (d, J=8.7 Hz, 2H), 8.16 (d, J=7.3 Hz, 2H), 7.97 (d, J=6.2 Hz, 2H), 7.84 (d, J=6.8 Hz, 2H), 7.79 (d, J=7.8 Hz, 2H), 7.61 (s, 2H), 7.56-7.45 (m, 4H), 7.42 (s, 2H), 7.30 (t, J=6.8 Hz, 2H), 4.83 (s, 1H), 2.34 (m, 2H), 1.88 (d, 4H), 1.71 (s, 5H), 1.53 (s, 1H), 1.31 (dd, J=14.8, 7.3 Hz, 4H), 1.16-1.06 (m, 2H), 0.79 (dd, J=14.6, 6.8 Hz, 4H), 0.66 (d, 12H), 0.50 (t, J=7.1 Hz, 6H), −0.23 (t, J=73 Hz, 6H).

### Synthesis of Compound La026

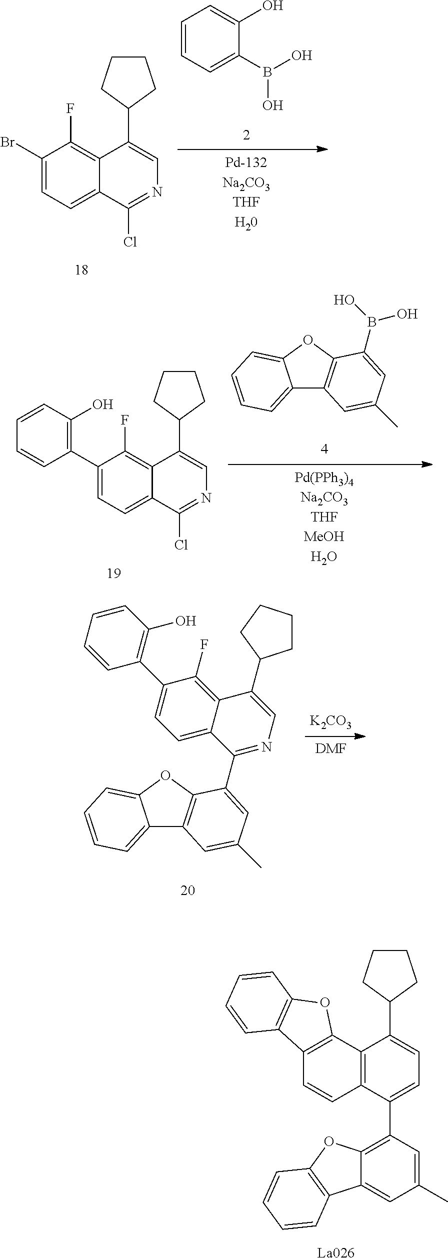

### Synthesis of Compound 19

Referring to the synthesis and purification method for Compound 3, only by changing the corresponding raw materials, target Compound 19 was obtained. Mass spectrum: 342.81 (M+H).

### Synthesis of Compound 20

Referring to the synthesis and purification method for Compound 5, only by changing the corresponding raw materials, target Compound 20 was obtained. Mass spectrum: 488.56 (M+H).

### Synthesis of Compound La026

Referring to the synthesis and purification method for Compound La001, only by changing the corresponding raw materials, target Compound La026 was obtained. Mass spectrum: 468.56 (M+H).

### Synthesis of Compound Ir(La026)₂(Lb008)

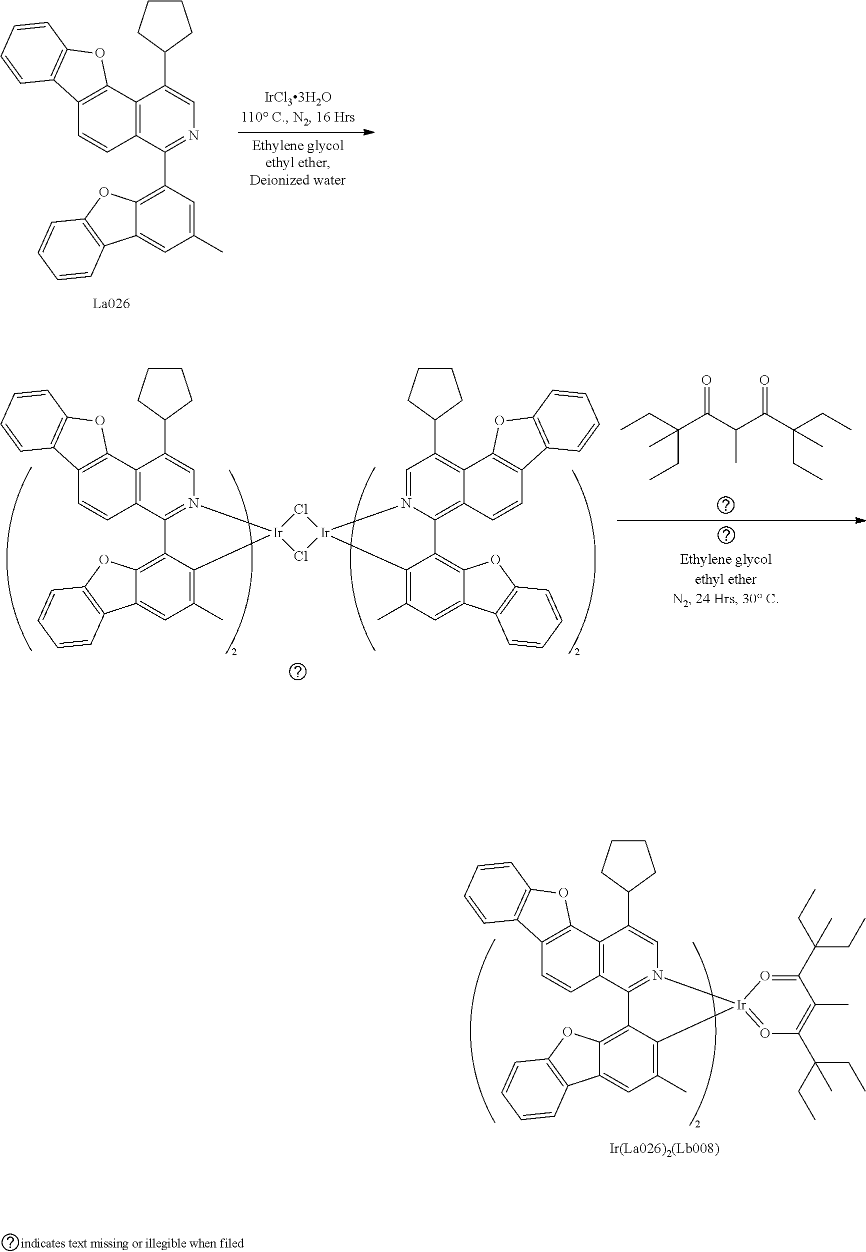

### Synthesis of Compound Ir(La026)-1

Referring to the synthesis and purification method for Compound Ir(La001)-1, by changing the corresponding raw materials, Compound Ir(La026)-1 was obtained which was directly used in the next step without purification.

### Synthesis of Compound Ir(La026)₂(Lb008)

Referring to the synthesis and purification method for Compound Ir(La001)₂(Lb005), only by changing the corresponding raw materials, Compound Ir(La026)₂(Lb008) was obtained as a red solid (2.74 g, yield: 41.43%). 2.74 g of crude Ir(La026)₂(Lb008) was sublimated and purified to obtain sublimated pure Ir(La026)₂(Lb008) (1.64 g, yield: 59.85%). Mass spectrum: 1379.72 (M+H). ¹H NMR (400 MHz, CDCl₃) δ 8.88 (s, 2H), 8.44 (d, J=6.3 Hz, 2H), 8.26 (d, J=7.7 Hz, 2H), 8.18 (d, J=7.2 Hz, 2H), 7.81 (d, J=7.3 Hz, 2H), 7.77 (d, J=8.2 Hz, 2H), 7.62 (t, J=6.8 Hz, 2H), 7.51-7.42 (m, 4H), 7.39 (s, 2H), 7.35 (t, J=7.8 Hz, 2H), 7.31 (t, J=7.0 Hz, 2H), 2.38 (m, 2H), 2.13 (s, 3H), 1.72 (s, 5H), 1.54 (s, 1H), 1.26 (dd, J=15.4, 7.0 Hz, 4H), 1.08 (m, 8H), 0.82 (dd, J=14.4, 6.7 Hz, 4H), 0.64 (s, 6H), 0.52 (t, J=7.4 Hz, 6H), −0.19 (t, J: 7.4 Hz, 6H).

### Synthesis of Compound La041

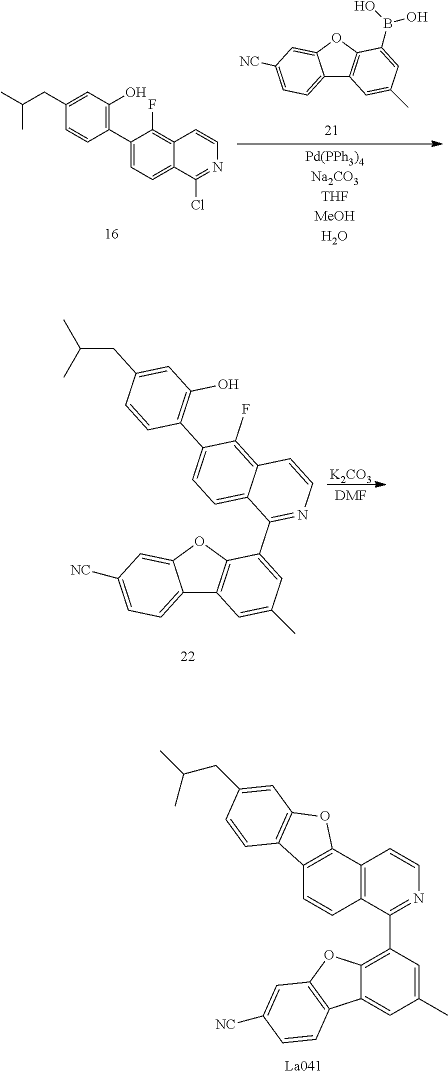

### Synthesis of Compound 22

Referring to the synthesis and purification method for Compound 5, only by changing the corresponding raw materials, target Compound 22 was obtained. Mass spectrum: 501.56 (M+H).

### Synthesis of Compound La041

Referring to the synthesis and purification method for Compound La001, only by changing the corresponding raw materials, target Compound La041 was obtained. Mass spectrum: 481.56 (M+H).

### Synthesis of Compound Ir(La041)₂(Lb031)

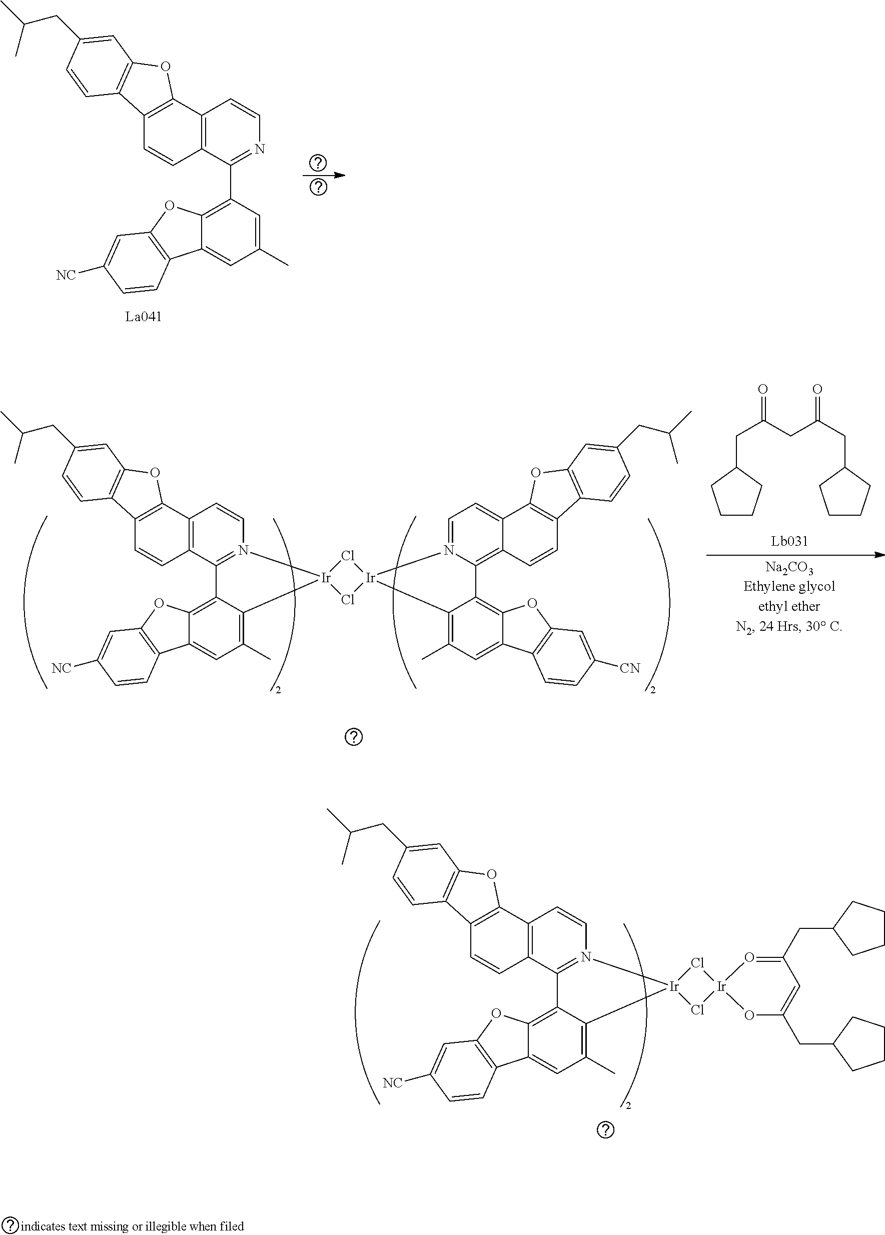

### Synthesis of Compound Ir(La041)-1

Referring to the synthesis and purification method for Compound Ir(La001)-1, by changing the corresponding raw materials, Compound Ir(La041)-1 was obtained which was directly used in the next step without purification.

### Synthesis of Compound Ir(La041)₂(Lb031)

Referring to the synthesis and purification method for Compound Ir(La001)₂(Lb005), only by changing the corresponding raw materials, Compound Ir(La041)₂(Lb031) was obtained as a red solid (2.69 g, yield: 40.23%). 2.69 g of crude Ir(La041)₂(Lb031) was sublimated and purified to obtain sublimated pure Ir(La041)₂(Lb031) (1.53 g, yield: 56.87%). Mass spectrum: 1387.65 (M+H). ¹H NMR (400 MHz, CDCl₃) δ 8.90 (d, J=9.2 Hz, 2H), 8.49 (d, J=6.3 Hz, 2H), 8.21 (d, J=8.7 Hz, 2H), 8.16 (d, J=7.3 Hz, 2H), 7.97 (d, J=6.2 Hz, 2H), 7.84 (d, J=6.8 Hz, 2H), 7.79 (d, J=7.8 Hz, 2H), 7.61 (s, 2H), 7.56-7.45 (m, 4H), 7.42 (s, 2H), 4.83 (s, 1H), 2.34 (m, 2H), 1.88 (d, 4H), 1.76 (m, 2H), 1.42 (dd, J=13.8, 7.6 Hz, 4H), 0.79 (dd, J=13.8, 6.6 Hz, 4H), 0.66 (d, 12H), 0.50 (t, J=7.1 Hz, 6H), 0.33 (m, 12H), 0.12 (m, 4H).

### Synthesis of Compound La052

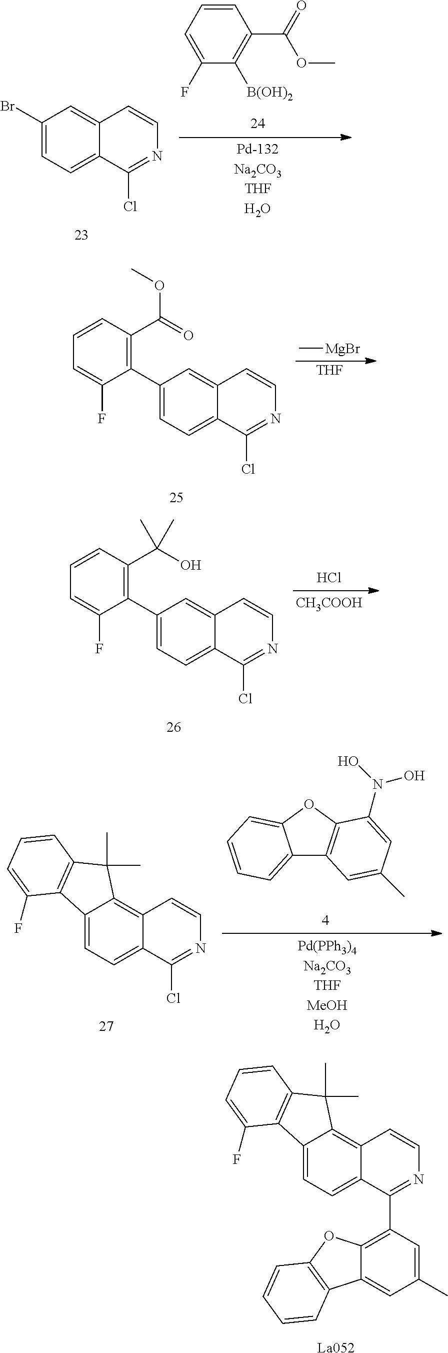

### Synthesis of Compound 25

Referring to the synthesis and purification method for Compound 3, only by changing the corresponding raw materials, target Compound 25 was obtained. Mass spectrum: 316.73 (M+H).

### Synthesis of Compound 26

Compound 25 (11.3 g, 35.79 mmol, 1.0 eq) and tetrahydrofuran (110 mL) were added to a 500 mL three-necked flask, and the flask was evacuated and displaced with nitrogen 3 times. Under nitrogen protection, 2 M methyl magnesium bromide (12.8 g, 107.37 mmol, 3.0 eq) was dropwise added slowly at 0° C. After the addition was completed, the temperature was raised to 50° C. and the reaction was conducted under stirring for 2 hours. TLC monitoring showed that Compound 25 was completely reacted. After cooling to room temperature, the reaction was quenched cy added with deionized water (110 mL), and ethyl acetate (150 mL) was then added for extraction and liquid separation. The organic phase was collected, dried by rotary evaporation, and then separated by column chromatography (the eluent was dichloromethane:n-hexane=1:10). After concentration, Compound 26 was obtained as a white solid (7.06 g, yield: 62.44%). Mass spectrum: 316.77 (M+H).

### Synthesis of Compound 27

Compound 26 (6.5 g, 20.58 mmol, 1.0 eq), a hydrochloric acid solution having a mass fraction of 36% (2.08 g, 20.58 mmol, 1.0 eq), and acetic acid (65 mL) were added to a 250 mL three-necked flask, and the flask was evacuated and displaced with nitrogen 3 times. Under nitrogen protection, the temperature was raised to 100° C. and the reaction was conducted under stirring for 4 hours. TLC monitoring showed that Compound 26 was completely reacted. After cooling to room temperature, deionized water (130 mL) was added and stirred, and ethyl acetate (150 mL) was then added for extraction and liquid separation. The organic phase was collected, dried by rotary evaporation, and then separated by column chromatography (the eluent was dichloromethane:n-hexane=1:15). After concentration, Compound 27 was obtained as a white solid (4.71 g, yield: 76.84%). Mass spectrum: 298.75 (M+H).

### Synthesis of Compound La052

Referring to the synthesis and purification method for Compound La001, only by changing the corresponding raw materials, target Compound La052 was obtained. Mass spectrum: 444.51 (M+H).

### Synthesis of Compound Ir(La052)₂(Lb005)

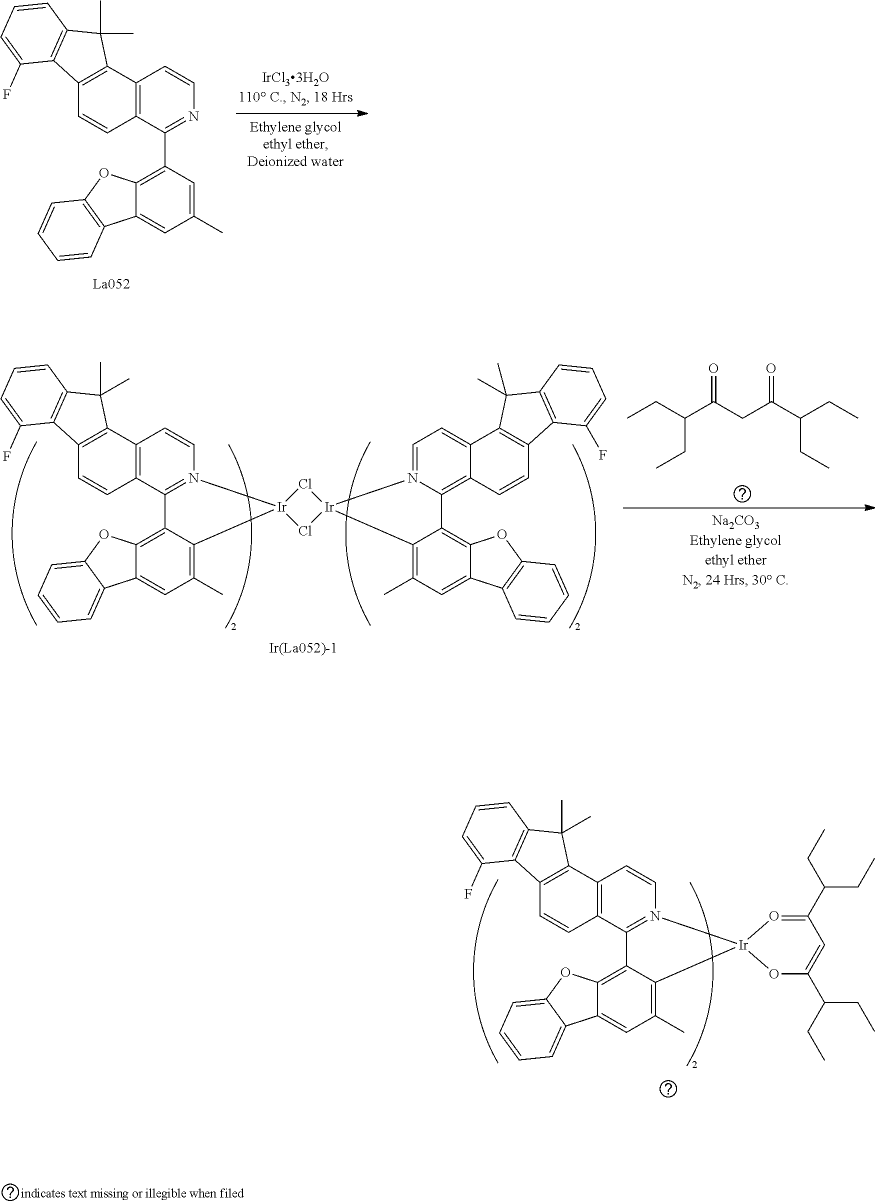

### Synthesis of Compound Ir(La052)-1

Referring to the synthesis and purification method for Compound Ir(La001)-1, by changing the corresponding raw materials, Compound Ir(La052)-1 was obtained which was directly used in the next step without purification.

### Synthesis of Compound Ir(La052)₂(Lb005)

Referring to the synthesis and purification method for Compound Ir(La001)₂(Lb005), only by changing the corresponding raw materials, Compound Ir(La052)₂(Lb005) was obtained as a red solid (2.14 g, yield: 38.64%). 2.14 g of crude Ir(La052)₂(Lb005) was sublimated and purified to obtain sublimated pure Ir(La052)₂(Lb005) (1.32 g, yield: 61.68%). Mass spectrum: 1289.54 (M+H). ¹H NMR (400 MHz, CDCl₃) δ 8.87 (d, J=8.8 Hz, 2H), 8.48 (d, J=6.8 Hz, 2H), 8.21 (d, J=7.3 Hz, 2H), 7.88 (d, J=6.8 Hz, 2H), 7.78 (d, J=6.8 Hz, 2H), 7.74 (d, J=7.8 Hz, 2H), 7.58 (t, J=6.5 Hz, 2H), 7.52-7.39 (m, 4H), 7.38 (s, 2H), 7.37 (t, J=7.6 Hz, 2H), 7.24 (t, J=7.0 Hz, 2H), 4.83 (s, 1H), 1.71 (s, 5H), 1.56 (s, 1H), 1.38 (dd, J=15.6, 7.0 Hz, 4H), 1.17-1.07 (m, 2H), 0.88 (dd, J=14.4, 6.7 Hz, 4H), 0.78 (s, 12H), 0.66 (t, J=7.4 Hz, 6H), −0.24 (t, J=7.4 Hz, 6H).

### Synthesis of Compound Ir(La052)₂(Lb008)

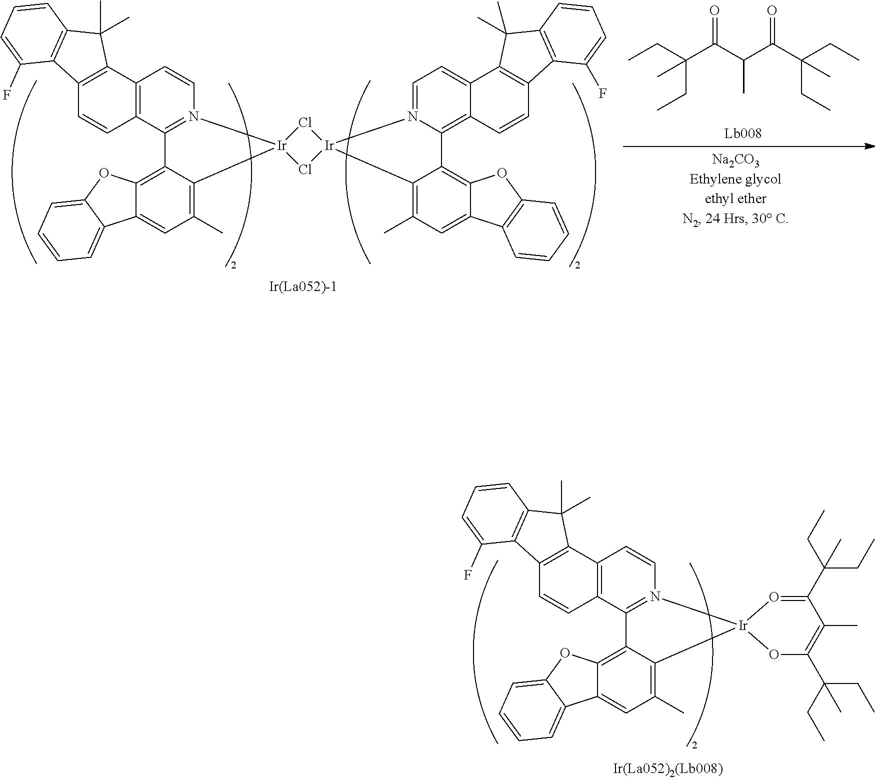

Referring to the synthesis and purification method for Compound Ir(La001)₂(Lb005), only by changing the corresponding raw materials, Compound Ir(La052)₂(Lb008) was obtained as a red solid (2.21 g, yield: 37.45%). 2.21 g of crude Ir(La052)₂(Lb008) was sublimated and purified to obtain sublimated pure Ir(La052)₂(Lb008) (1.26 g, yield: 57.01%). Mass spectrum: 1331.62 (M+H). ¹H NMR (400 MHz, CDCl₃) δ 8.90 (d, J=8.6 Hz, 2H), 8.53 (d, J=6.8 Hz, 2H), 8.27 (d, J=7.3 Hz, 2H), 7.89 (d, J=6.8 Hz, 2H), 7.79 (d, J=6.8 Hz, 2H), 7.74 (d, J=7.8 Hz, 2H), 7.58 (t, J=6.5 Hz, 2H), 7.52-7.39 (m, 4H), 7.38 (s, 2H), 7.37 (t, J=7.6 Hz, 2H), 7.24 (t, J=7.0 Hz, 2H), 4.83 (s, 1H), 1.71 (s, 6H), 1.16 (s, 6H), 1.08 (s, 3H), 0.91 (m, 8H), 0.78 (s, 12H), 0.66 (m, 12H).

### Synthesis of Compound La078

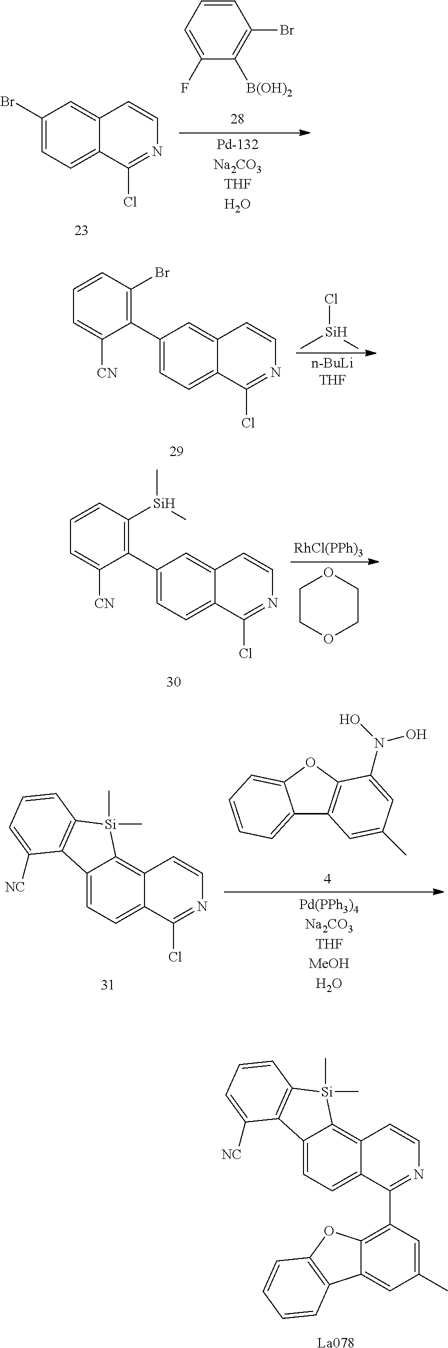

### Synthesis of Compound 29

Referring to the synthesis and purification method for Compound 3, only by changing the corresponding raw materials, target Compound 29 was obtained. Mass spectrum: 344.61 (M+H).

### Synthesis of Compound 30

Compound 30 (13.2 g, 38.42 mmol, 1.0 eq) and tetrahydrofuran (132 mL) were added to a 500 mL three-necked flask, and the flask was evacuated and displaced with nitrogen 3 times. Under nitrogen protection, 1.5 M n-butyllithium (30.73 mL, 46.1 mmol, 1.2 eq) was dropwise added slowly at −78° C. After the addition was completed, the mixture was stirred for 1 h, dimethyl monochlorosilane (5.45 g, 57.62 mmol, 1.5 eq) was then dropwise added slowly, and after the dropwise addition was completed, the mixture was returned to room temperature and stirred for 2 hours. TLC monitoring showed that Compound 30 was completely reacted. Deionized water (130 ml) was slowly added for quenching the reaction, and ethyl acetate (150 mL) was then added for extraction and liquid separation. The organic phase was collected, dried by rotary evaporation, and then separated by column chromatography (the eluent was dichloromethane:n-hexane=1:20). After concentration, Compound 30 was obtained as a white solid (8.76 g, yield: 70.62%). Mass spectrum: 323.86 (M+H).

### Synthesis of Compound 31

Compound 30 (8.0 g, 24.78 mmol, 1.0 eq), triphenylphosphine rhodium chloride (0.22 g, 0.24 mmol, 0.01 eq), and dioxane (80 mL) were added to a 250 mL three-necked flask, and the flask was evacuated and displaced with nitrogen 3 times. Under nitrogen protection, the temperature was raised to 130° C., and the reaction was conducted under stirring for 2 hours. TLC monitoring showed that Compound 30 was completely reacted. After cooling to room temperature, the organic phase was dried by rotary evaporation and separated by column chromatography (the eluent was ethyl acetate:n-hexane=1:20). After concentration, Compound 31 was obtained as a white solid (5.75 g, yield: 72.3%). Mass spectrum: 321.85 (M+H).

### Synthesis of Compound La078

Referring to the synthesis and purification method for Compound La001, only by changing the corresponding raw materials, target Compound La078 was obtained. Mass spectrum: 467.6 (M+H).

### Synthesis of Compound Ir(La078)₂(Lb005)

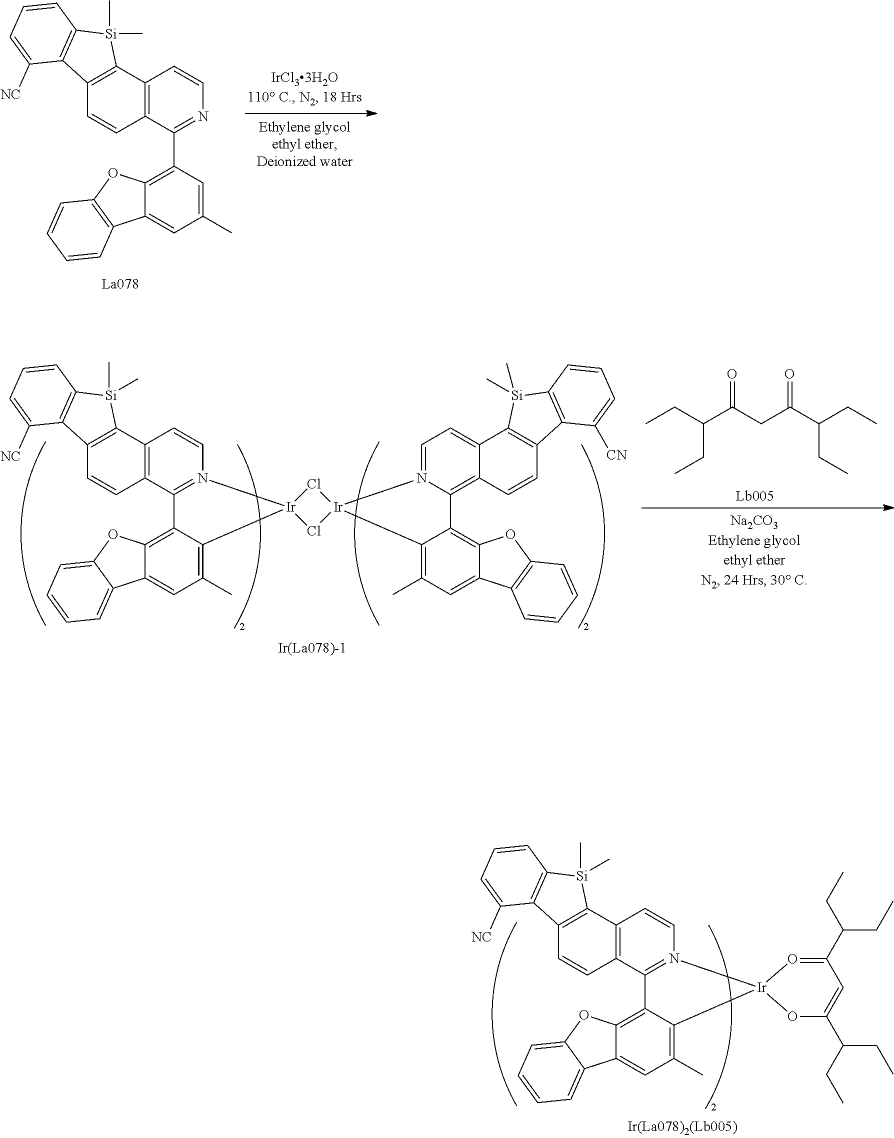

### Synthesis of Compound Ir(La078)-1

Referring to the synthesis and purification method for Compound Ir(La001)-1, by changing the corresponding raw materials, Compound Ir(La078)-1 was obtained which was directly used in the next step without purification.

### Synthesis of Compound Ir(La078)₂(Lb005)

Referring to the synthesis and purification method for Compound Ir(La001)₂(Lb005), only by changing the corresponding raw materials, Compound Ir(La078)₂(Lb005) was obtained as a red solid (2.62 g, yield: 36.63%). 2.62 g of crude Ir(La078)₂(Lb005) was sublimated and purified to obtain sublimated pure Ir(La078)₂(Lb005) (1.48 g, yield: 56.48%). Mass spectrum: 1335.73 (M+H). ¹H NMR (400 MHz, CDCl₃) δ 8.90 (d, J=8.6 Hz, 2H), 8.42 (d, J=6.2 Hz, 2H), 8.23 (d, J=7.5 Hz, 2H), 7.86 (d, J=7.2 Hz, 2H), 7.78 (d, J=7.4 Hz, 2H), 7.76 (d, J=6.6 Hz, 2H), 7.61 (t, J=6.5 Hz, 2H), 7.53-7.40 (m, 4H), 7.38 (s, 2H), 7.32 (t, J=7.6 Hz, 2H), 7.22 (t, J=7.0 Hz, 2H), 4.83 (s, 1H), 1.71 (s, 5H), 1.56 (s, 1H), 1.38 (dd, J=15.6, 7.0 Hz, 4H), 1.17-1.07 (m, 2H), 0.88 (dd, J=14.4, 6.7 Hz, 4H), 0.72 (s, 12H), 0.66 (t, J=7.4 Hz, 6H), −0.24 (t, J=7.4 Hz, 6H).

### Synthesis of Compound Ir(La078)₂(Lb008)

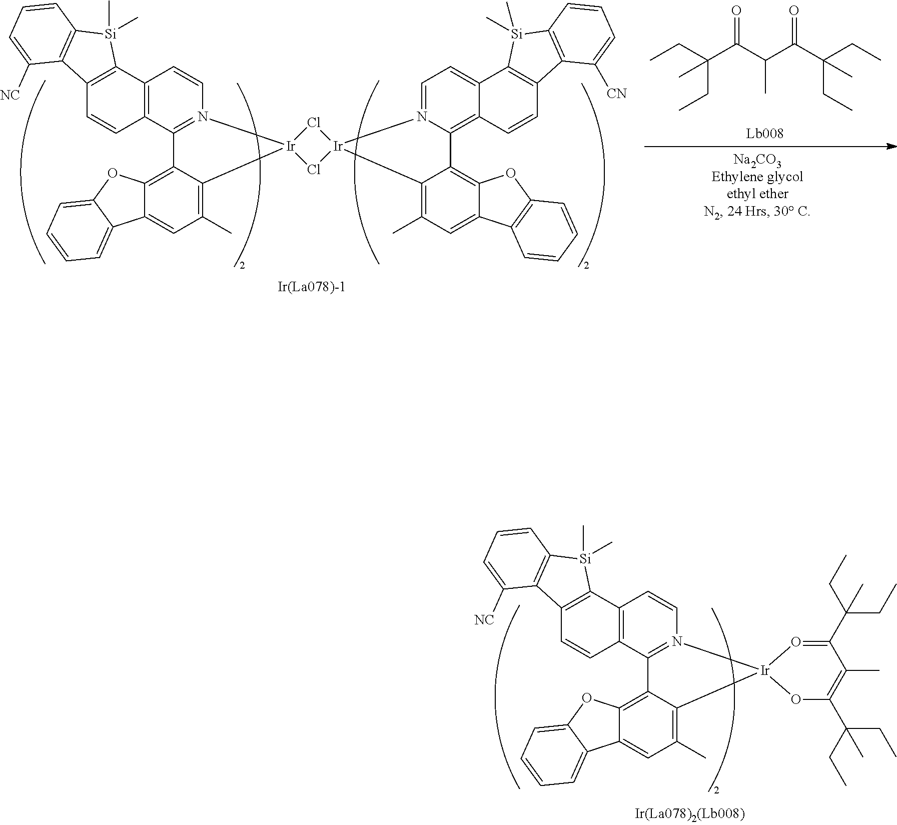

Referring to the synthesis and purification method for Compound Ir(La001)₂(Lb005), only by changing the corresponding raw materials, Compound Ir(La078)₂(Lb008) was obtained as a red solid (2.53 g, yield: 37.23%). 2.53 g of crude Ir(La078)₂(Lb008) was sublimated and purified to obtain sublimated pure Ir(La078)₂(Lb008) (1.26 g, yield: 49.8%). Mass spectrum: 1377.81 (M+H). ¹H NMR (400 MHz, CDCl₃) δ 8.89 (d, J=8.6 Hz, 2H), 8.54 (d, J=7.2 Hz, 2H), 8.21 (d, J=7.6 Hz, 2H), 7.82 (d, J=6.6 Hz, 2H), 7.76 (d, J=6.2 Hz, 2H), 7.68 (d, J=7.8 Hz, 2H), 7.54 (t, J=6.7 Hz, 2H), 7.52-7.39 (m, 4H), 7.39 (s, 2H), 7.35 (t, J=7.6 Hz, 2H), 7.25 (t, J=7.0 Hz, 2H), 4.81 (s, 1H), 1.75 (s, 6H), 1.13 (s, 6H), 1.12 (s, 3H), 0.88 (m, 8H), 0.73 (s, 12H), 0.64 (m, 12H).

### Synthesis of Compound Lc004

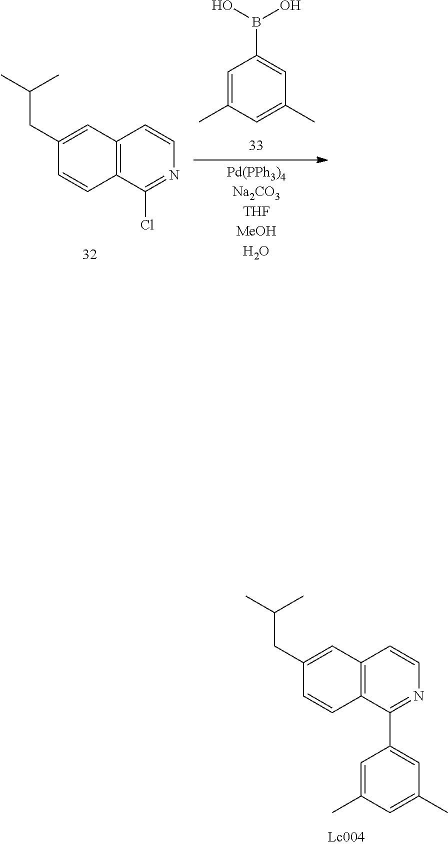

Referring to the synthesis and purification method for Compound La001, only by changing the corresponding raw materials, target Compound Lc004 was obtained. Mass spectrum: 290.41 (M+H).

### Synthesis of Compound Ir(La078)(Lb005)(Lc004)

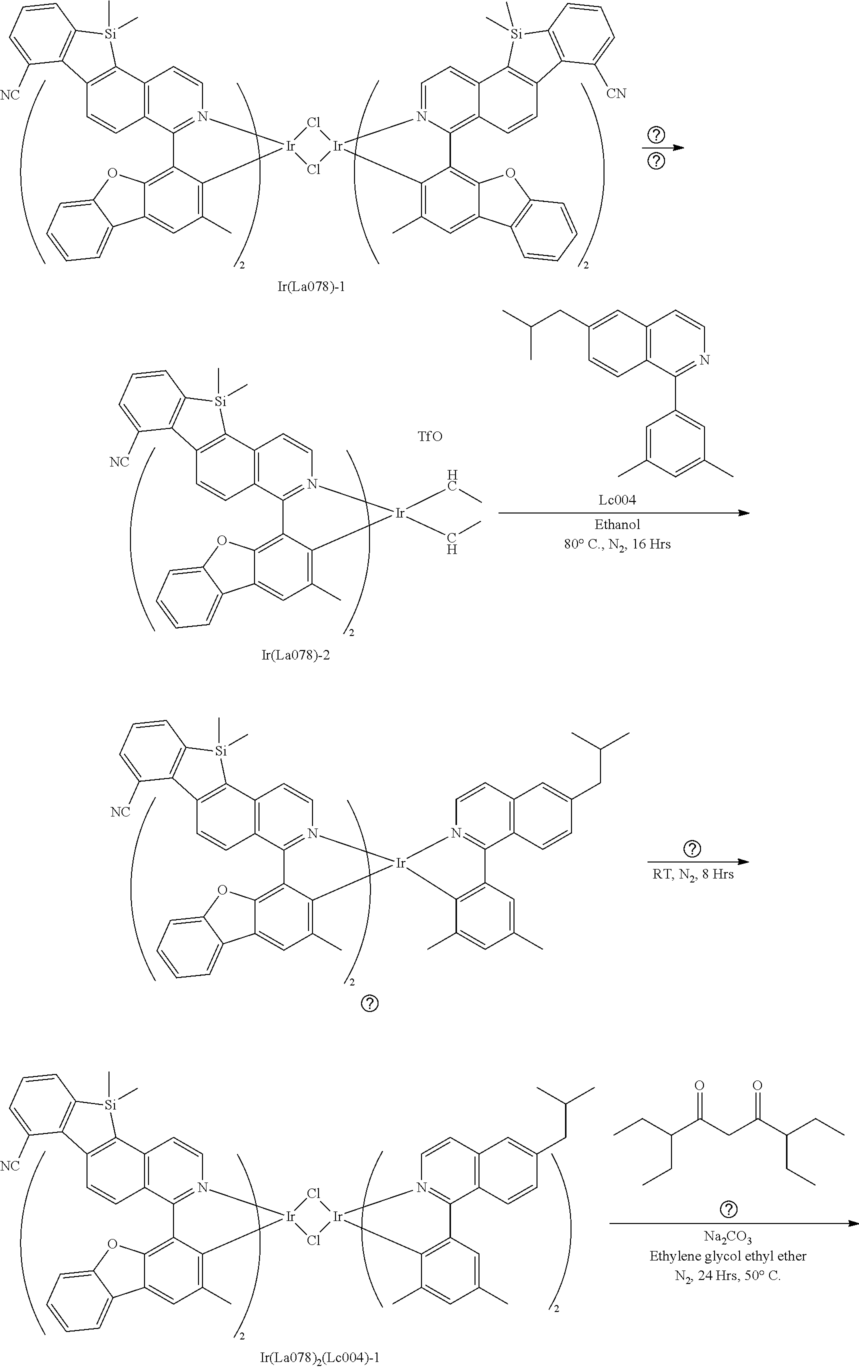

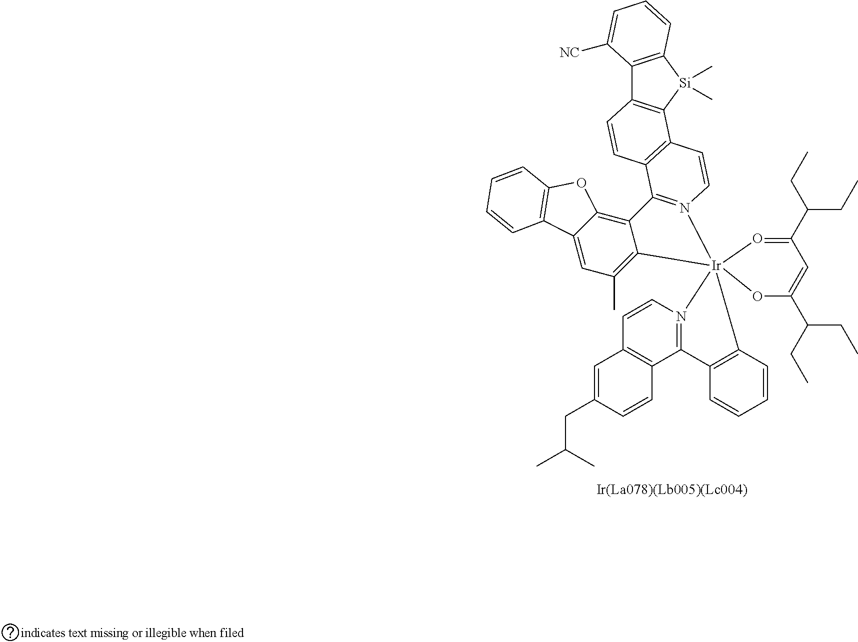

### Synthesis of Compound Ir(La078)-2

Dimer Ir(La078)-1 (7.65 g, 6.6 mmol, 1.0 eq) and dichloromethane (574 mL) were added to a 3 L three-necked flask and stirred for dissolution. Silver trifluoromethanesulfonate (3.39 g, 13.2 mmol, 2.0 eq) was dissolved in methanol (380 mL) and then added to the solution in the original reaction flask, the flask was evacuated and displaced with nitrogen 3 times. The resulting mixed solution was stirred at room temperature for 16 hours under N₂ protection. Then, the reaction solution was filtered with diatomite, the filter residue was washed with dichloromethane (150 mL), and the filtrate was dried by rotary evaporation to obtain Compound Ir(La078)-2 (6.96 g, 78.84%). The obtained compound could be directly used in the next step without purification.

### Synthesis of Compound Ir(La078)₂(Lc004)

Compound Ir(La078)-2 (6.85 g, 5.13 mmol, 1.0 eq) and Lc004 (3.71 g, 12.81 mmol, 2.5 eq) were added to a 250 mL three-necked flask, ethanol (102 mL) was added, and the flask was evacuated and displaced with nitrogen 3 times. Under N₂ protection, the mixture was refluxed under stirring for 16 hours. After cooling to room temperature, filtration was carried out. A solid was collected, dissolved with dichloromethane (180 mL), and filtered by silica gel, and the filter cake was then sprinkled with dichloromethane (80 mL). The filtrate was dried by rotary evaporation, recrystallized twice with tetrahydrofuran/methanol (product:tetrahydrofuran:methanol=1:8:10), and dried to obtain Compound Ir(La078)₂(Lc004) (3.52 g, 48.66%). Mass spectrum: 1412.82 (M+H).

### Synthesis of Compound Ir(La078)₂(Lc004)-1

Compound Ir(La078)₂(Lc004) (4.2 g, 2.97 mmol, 1.0 eq) and zinc chloride (20.27 g, 148.4 mmol, 50 eq) were placed in a 1 L single-necked flask, 1,2-dichloroethane (210 mL) was added, and the flask was evacuated and displaced with nitrogen 3 times. Under N₂ protection, the reflux reaction was conducted under stirring for 18 hours. TLC spotting monitoring showed that raw material Ir(La078)₂(Lc004) was basically completely reacted. After cooling to room temperature, deionized water was added for washing 3 times (100 mL once), and the filtrate was dried by rotary evaporation to obtain Compound Ir(La078)₂(Lc004)-1 (2.36 g, 80.67%). The obtained compound could be directly used in the next step without purification.

### Synthesis of Compound Ir(La078)(Lb005)(Lb004)

Compound Ir(La078)₂(Lc004)-1(3.5 g, 3.57 mmol, 1.0 eq), Lb005 (3.79 g, 17.83 mmol, 5.0 eq), and sodium carbonate (3.78 g, 35.65 mmol, 10.0 eq) were placed in a 250 mL single-necked round-bottom flask, ethylene glycol ethyl ether (52 mL) was added, the flask was evacuated and displaced with nitrogen 3 times. The resulting mixed solution was stirred at 50° C. for 24 hours under N₂ protection. TLC monitoring showed that Ir(La078)₂(Lc004)-1 was completely reacted. After cooling to room temperature, the reaction solution was added with 104 mL of methanol for pulping at room temperature for 2 h, and subjected to suction filtration. The filter cake was dissolved with dichloromethane (100 mL) and filtered with silica gel. The filter cake was then sprinkled with dichloromethane (50 mL), the filtrate was collected, and deionized water was added for washing 3 times (60 Ml once). After liquid separation, the organic phase was collected, concentrated and dried to obtain a dark red solid, which was recrystallized 3 times with tetrahydrofuran/methanol (product:tetrahydrofuran:methanol=1:8:10) to obtain Compound Ir(La078)(Lb005)(Lb004) as a red solid (1.9 g, yield: 46.71%). 1.9 g of crude Ir(La078)(Lb005)(Lb004) was sublimated and purified to obtain sublimated pure Ir(La078)(Lb005)(Lb004) (0.96 g, yield: 50.52%). Mass spectrum: 1158.45 (M+H). ¹HNMR (400 MHz, CDCl₃) δ 8.87 (d, 1H), 8.45 (d, 1H), 8.27 (d, 1H), 8.07 (d, 1H), 7.95 (m, 3H), 7.78 (d, 1H), 7.69 (d, J=5.0 Hz, 2H), 7.60 (d, 1H), 7.57-7.48 (m, 5H), 7.39 (d, 1H), 7.31 (d, 1H), 6.92 (d, 1H), 4.81 (s, 1H), 2.43 (d, 2H), 2.32 (d, J=15.0 Hz, 6H), 1.82 (m, 1H), 1.27 (m, 8H), 1.01 (m, 5H), 0.94 (m, 12H), 0.87 (d, 6H), 0.66 (s, 6H).

### Synthesis of Compound Lc024

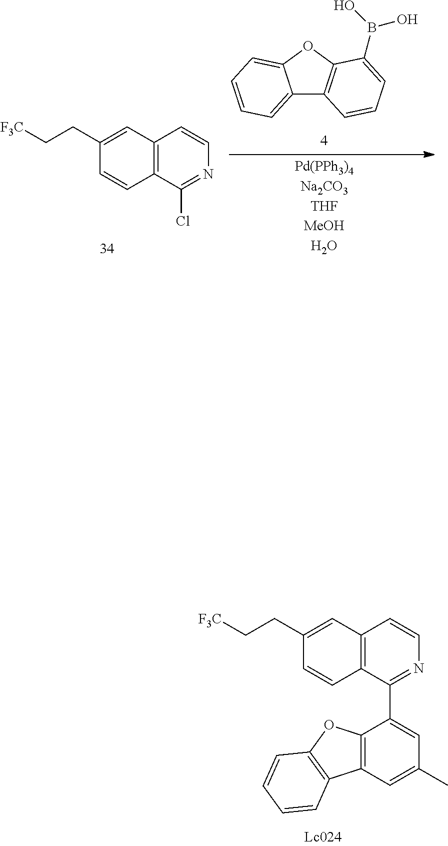 [SMILES: OB(O)c1cccc2c1oc1ccccc12 ; FC(F)(F)CCc1ccc2c(Cl)nccc2c1 ; Cc1cc(-c2nccc3cc(CCC(F)(F)F)ccc23)c2oc3ccccc3c2c1]

Referring to the synthesis and purification method for Compound La001, only by changing the corresponding raw materials, target Compound Lc024 was obtained. Mass spectrum: 406.41 (M+H).

### Synthesis of Compound Ir(La078)(Lb005)(Lc024)

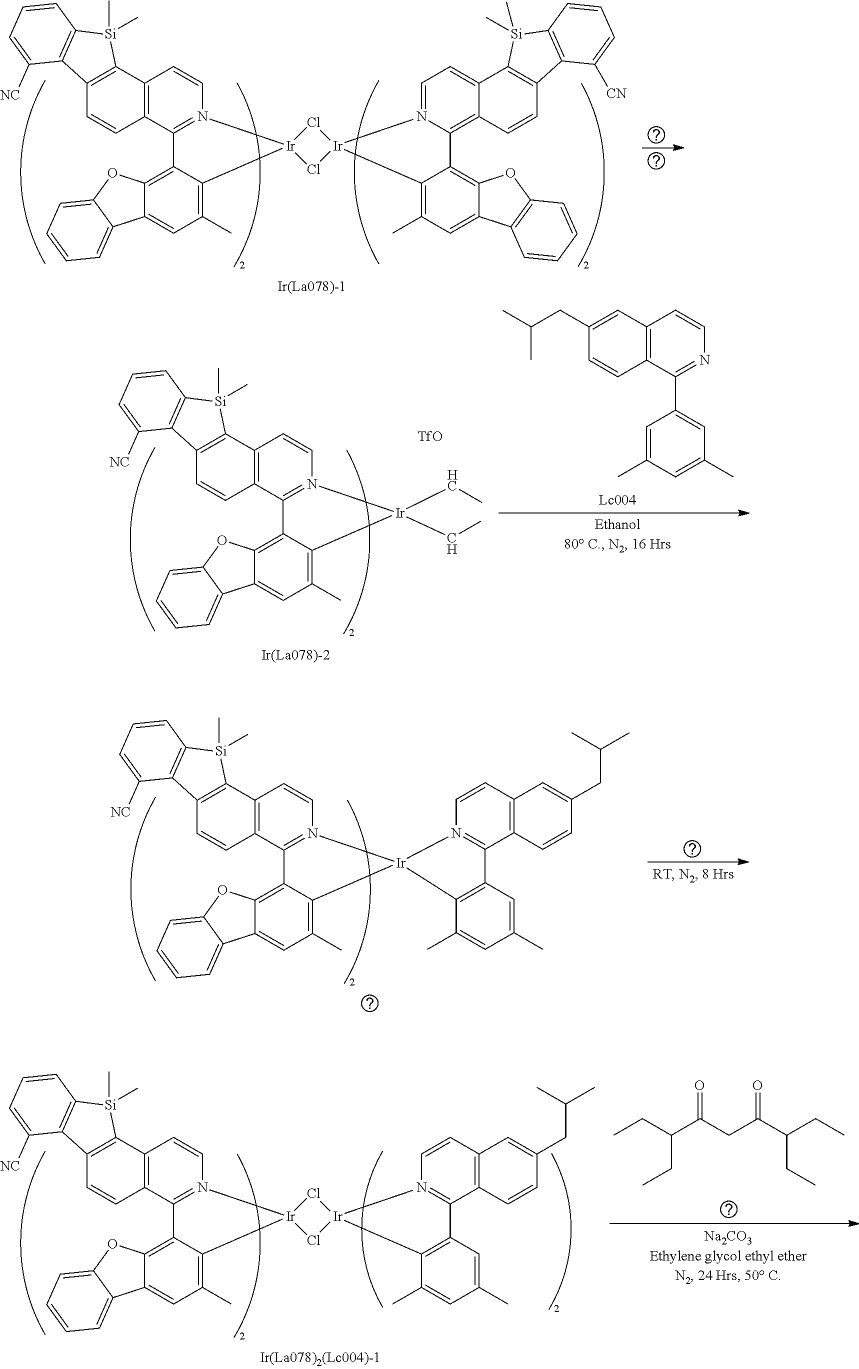

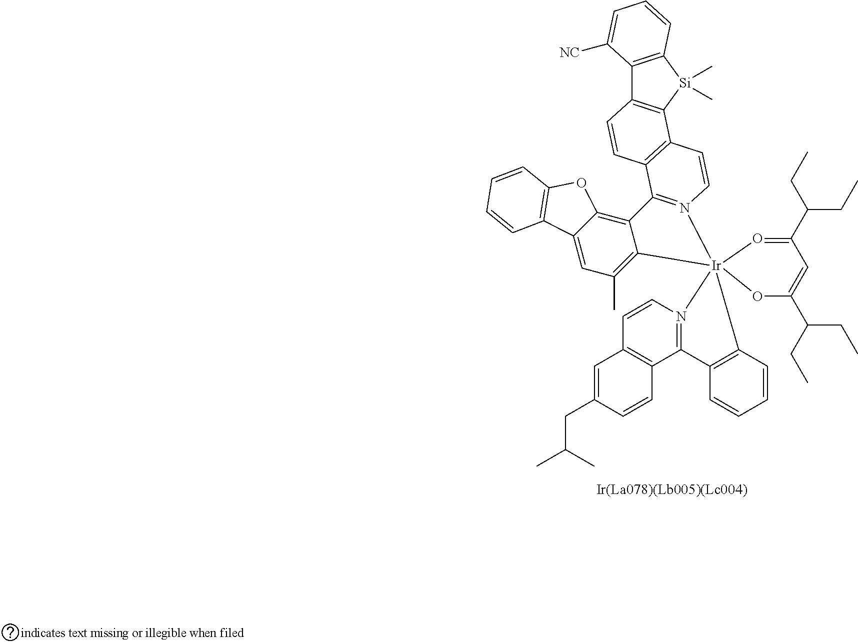

### Synthesis of Compound Ir(La078)₂(Lc024)

Referring to the synthesis and purification method for Compound Ir(La078)₂(Lc004), only by changing the corresponding raw materials, target Compound Ir(La078)₂(Lc024) was obtained. Mass spectrum: 1558.71 (M+H).

### Synthesis of Compound Ir(La078)₂(Lc024)-1

Referring to the synthesis and purification method for Compound Ir(La078)₂(Lc004)-1, by changing the corresponding raw materials, Compound Ir(La078)₂(Lc024)-1 was obtained which was directly used in the next step without purification.

### Synthesis of Compound Ir(La078)(Lb005)(Lc024)

Referring to the synthesis and purification method for Compound Ir(La078)(Lb005)(Lc004), only by changing the corresponding raw materials, Compound Ir(La078)(Lb005)(Lc024) was obtained as a red solid (2.27 g, yield: 36.27%). 2.27 g of crude Ir(La078)(Lb005)(Lc024) was sublimated and purified to obtain sublimated pure Ir(La078)(Lb005)(Lc024) (1.14 g, yield: 50.22%). Mass spectrum: 1274.51 (M+H). ¹HNMR (400 MHz, CDCl₃) δ 8.86 (d, 1H), 8.42 (d, 1H), 8.27 (s, 1H), 8.02-7.89 (m, 3H), 7.81-7.65 (m, 4H), 7.64-7.48 (m, 4H), 7.42 (d, J=30.0 Hz, 2H), 7.36 (m, 3H), 7.31 (s, 1H), 7.21 (m, 2H), 4.81 (s, 1H), 2.63 (t, 2H), 2.50 (s, 3H), 1.89 (m, 2H), 1.66 (d, 2H), 1.27 (m, 8H), 1.09-0.88 (m, 15H), 0.66 (s, 6H).

### Synthesis of Compound Lc025

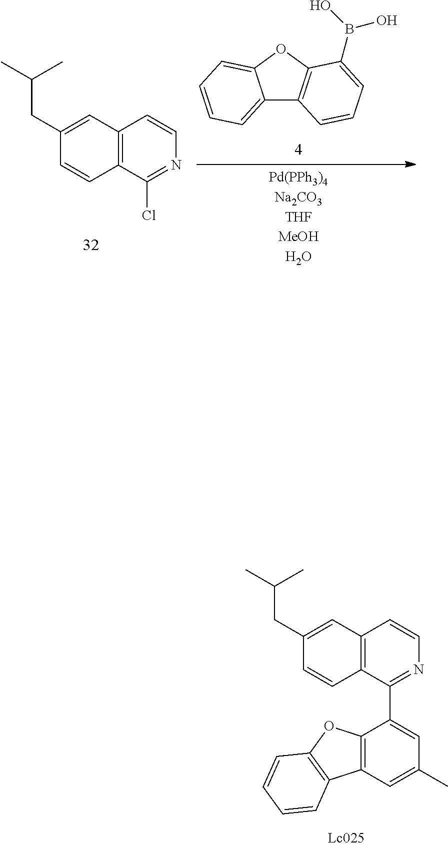

Referring to the synthesis and purification method for Compound La001, only by changing the corresponding raw materials, target Compound Lc025 was obtained. Mass spectrum: 366.47 (M+H).

### Synthesis of Compound Ir(La078)(Lb005)(Lc025)

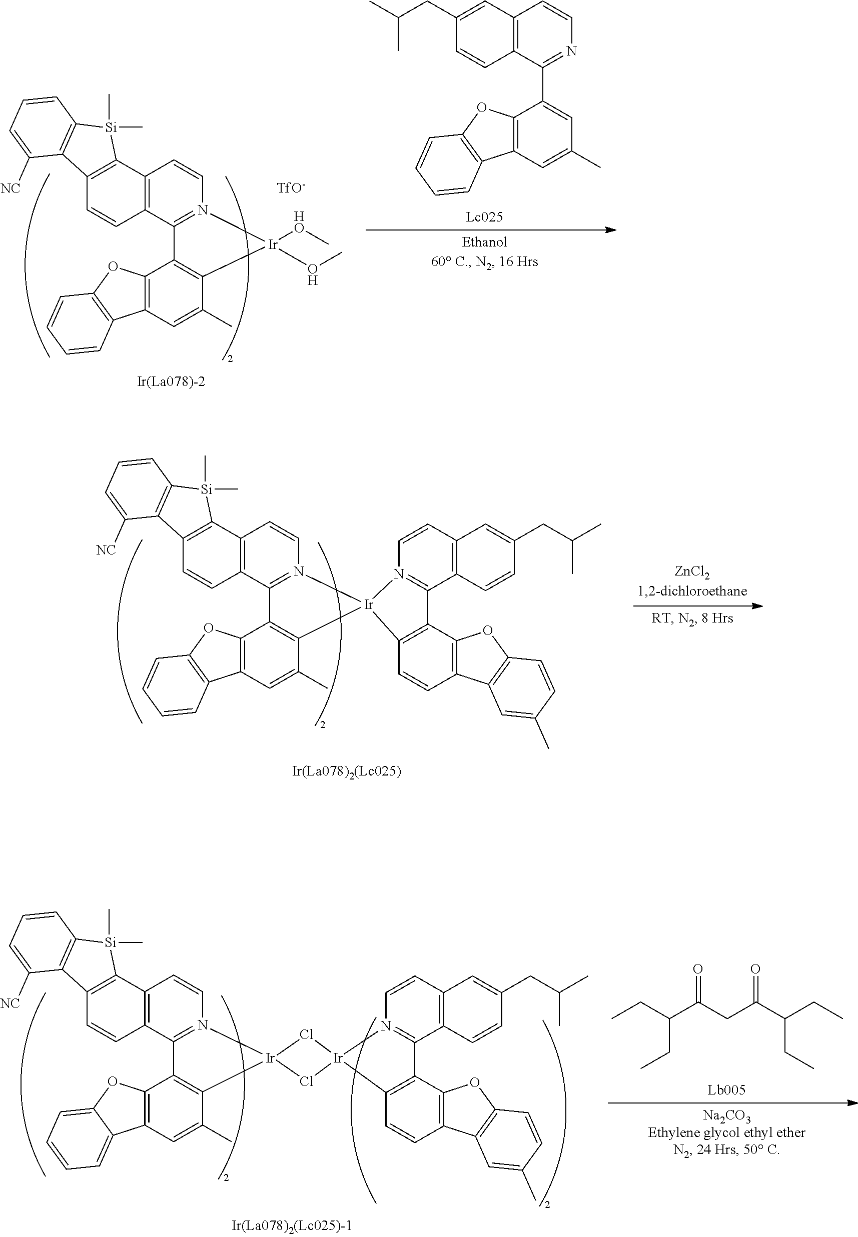

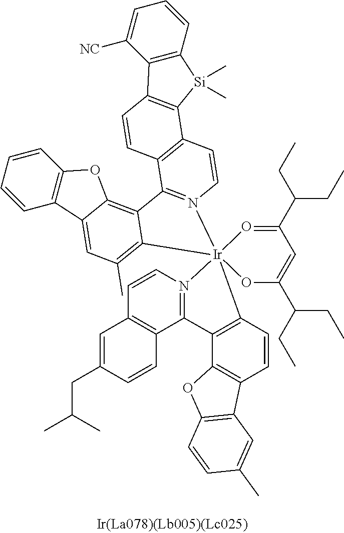

### Synthesis of Compound Ir(La078)₂(Lc025)

Referring to the synthesis and purification method for Compound Ir(La078)₂(Lc004), only by changing the corresponding raw materials, target Compound Ir(La078)₂(Lc025) was obtained. Mass spectrum: 1488.87 (M+H).

### Synthesis of Compound Ir(La078)₂(Lc025)-1

Referring to the synthesis and purification method for Compound Ir(La078)₂(Lc004)-1, by changing the corresponding raw materials, Compound Ir(La078)₂(Lc025)-1 was obtained which was directly used in the next step without purification.

### Synthesis of Compound Ir(La078)(Lb005)(Lc025)

Referring to the synthesis and purification method for Compound Ir(La078)(Lb005)(Lc004), only by changing the corresponding raw materials, Compound Ir(La078)(Lb005)(Lc025) was obtained as a red solid (2.46 g, yield: 39.65%). 2.46 g of crude Ir(La078)(Lb005)(Lc025) was sublimated and purified to obtain sublimated pure Ir(La078)(Lb005)(Lc025) (1.45 g, yield: 54.87%). Mass spectrum: 1234.59 (M+H). ¹HNMR (400 MHz, CDCl₃) δ 8.88 (d, 1H), 8.45 (d, 1H), 8.24 (s, 1H), 8.10-7.92 (m, 3H), 7.82-7.66 (m, 4H), 7.62-7.46 (m, 4H), 7.42 (d, J=30.0 Hz, 2H), 7.38 (m, 3H), 7.32 (s, 1H), 7.21 (m, 2H), 4.81 (s, 1H), 2.63 (t, 2H), 2.50 (s, 3H), 2.32 (m, 1H) 1.89 (m, 2H), 1.66 (d, 2H), 1.34 (d, 6H), 1.27 (m, 8H), 1.09-0.88 (m, 15H), 0.66 (s, 6H), 0.23 (m, 4H).

### Example of Use: Manufacturing of Organic Electroluminescent Device

A 50 mm*50 mm*1.0 mm glass substrate with an ITO (70 Å/1000 Å/110 Å) anode electrode was ultrasonically cleaned in ethanol for 10 minutes, then dried at 150° C. and then treated with N₂ Plasma for 30 minutes. The washed glass substrate was arranged on a substrate holder of a vacuum evaporation device. Firstly, compound HTM1 and P-dopant (at a ratio of 97%:3%) were evaporated on the side of the glass substrate, on which there was an anode electrode wire, by covering the electrode in a co-evaporation manner to form a thin film with a thickness of 100 Å, followed by immediately evaporation of a layer of HTM1 to form a thin film with at thickness of about 1720 Å, and then evaporation of a layer of HTM2 on the HTM1 thin film to form a thin film with a thickness of about 100 Å. Then, Host Material 1, Host Material 2, and a doping compound (at a ratio of 48.5%:48.5%:3%, Comparative Compound X or the compound of the present disclosure) were evaporated on the HTM2 film layer by means of co-evaporation again to form a film with a thickness of 400 Å. The ratio of the host materials to the doping material was 90%: 10%. ETL:LiQ (350 Å, the ratio was 50%:50%) was evaporated on the luminous layer by means of co-evaporation. Yb (10 Å) was then evaporated on the electron transport layer material, and finally a layer of metal Ag (150 Å) was evaporated as an electrode.

| | H1L | HTL | EBL | Luminous | Electron |
|---|---|---|---|---|---|
| | Thick- | Thick- | Thick- | layer | transport |
| | ness/ | ness/ | ness/ | Thickness/ | layer |
| Example | Å | Å | Å | Å | Thickness/Å |
| A1 | HTM1: | HTM1 | HTM2 | H1:H2: | ETL:LIQ |
| | NDP-9 | 1720 | 100 | Ir(La001)₂ | 350 |
| | | | | (Lb005) | |
| | 100 | | | 400 | |
| A2 | HTM1: | HTM1 | HTM2 | H1:H2: | ETL:LIQ |
| | NDP-9 | 1720 | 100 | Ir(La005)₂ | 350 |
| | 100 | | | (Lb005) | |
| | | | | 400 | |
| A3 | HTM1: | HTM1 | HTM2 | H1:H2:Ir | ETL:LIQ |
| | NDP-9 | 1720 | 100 | (La007)₂ | 350 |
| | 100 | | | (Lb005) | |
| | | | | 400 | |
| A4 | HTM1: | HTM1 | HTM2 | H1:H2:Ir | ETL:LIQ |
| | NDP-9 | 1720 | 100 | (La011)₂ | 350 |
| | 100 | | | (Lb005) | |
| | | | | 400 | |
| A5 | HTM1: | HTM1 | HTM2 | H1:H2:Ir | ETL:LIQ |
| | NDP-9 | 1720 | 100 | (La014)₂ | 350 |
| | 100 | | | (Lb005) | |
| | | | | 400 | |
| A6 | HTM1: | HTM1 | HTM2 | H1:H2:Ir | ETL:LIQ |
| | NDP-9 | 1720 | 100 | (La026)₂ | 350 |
| | 100 | | | (Lb008) | |
| | | | | 400 | |
| A7 | HTM1: | HTM1 | HTM2 | H1:H2:Ir | ETL:LIQ |
| | NDP-9 | 1720 | 100 | (La041)₂ | 350 |
| | 100 | | | (Lb031) | |
| | | | | 400 | |
| A8 | HTM1: | HTM1 | HTM2 | H1:H2:Ir | ETL:LIQ |
| | NDP-9 | 1720 | 100 | (La052)₂ | 350 |
| | 100 | | | (Lb005) | |
| | | | | 400 | |
| A9 | HTM1: | HTM1 | HTM2 | H1:H2:Ir | ETL:LIQ |
| | NDP-9 | 1720 | 100 | (La052)₂ | 350 |
| | 100 | | | (Lb008) | |
| | | | | 400 | |
| A10 | HTM1: | HTM1 | HTM2 | H1:H2:Ir | ETL:LiQ |
| | NDP-9 | 1720 | 100 | (La078)₂ | 350 |
| | 100 | | | (Lb005) | |
| | | | | 400 | |
| A11 | HTM1: | HTM1 | HTM2 | H1:H2:Ir | ETL:LIQ |
| | NDP-9 | 1720 | 100 | (La078)₂ | 350 |
| | 100 | | | (Lb008) | |
| | | | | 400 | |
| A12 | HTM1: | HTM1 | HTM2 | H1:H2 | ETL:LIQ |
| | NDP-9 | 1720 | 100 | Ir(La078) | 350 |
| | 100 | | | (Lb005) | |
| | | | | (Lc004) | |
| | | | | 400 | |
| A13 | HTM1: | HTM1 | HTM2 | H1:H2 | ETL:LiQ |
| | NDP-9 | 1720 | 100 | Ir(La078) | 350 |
| | 100 | | | (Lb005) | |
| | | | | (Lc024) | |
| | | | | 400 | |
| A14 | HTM1: | HTM1 | HTM2 | H1:H2 | ETL:LIQ |
| | NDP-9 | 1720 | 100 | Ir(La078) | 350 |
| | 100 | | | (Lb005) | |
| | | | | (Lc025) | |
| | | | | 400 | |
| Comparative | HTM1: | HTM1 | HTM2 | H1:H2: | ETL:LIQ |
| Example 1 | NDP-9 | 1720 | 100 | Comparative | 350 |
| | 100 | | | Compound 1 | |
| | | | | 400 | |
| Comparative | HTM1: | HTM1 | HTM2 | H1:H2 | ETL:LIQ |
| Example 2 | NDP-9 | 1720 | 100 | Comparative | 350 |
| | 100 | | | Compound 2 | |
| | | | | 400 | |
| Comparative | HTM1: | HTM1 | HTM2 | H1:H2 | ETL:LiQ |
| Example 3 | NDP-9 | 1720 | 100 | Comparative | 350 |
| | 100 | | | Compound 3 | |
| | | | | 400 | |
| Comparative | HTM1: | HTM1 | HTM2 | H1:H2: | ETL:LIQ |
| Example 4 | NDP-9 | 1720 | 100 | Comparative | 350 |
| | 100 | | | Compound 4 | |
| | | | | 400 | |
| Comparative | HTM1: | HTM1 | HTM2 | H1:H2: | ETL:LIQ |
| Example 5 | NDP-9 | 1720 | 100 | Comparative | 350 |
| | 100 | | | Compound 5 | |
| | | | | 400 | |
| Comparative | HTM1: | HTM1 | HTM2 | H1:H2: | ETL:LiQ |
| Example 6 | NDP-9 | 1720 | 100 | Comparative | 350 |
| | 100 | | | Compound 6 | |
| | | | | 400 | |
| Comparative | HTM1: | HTM1 | HTM2 | H1:H2: | ETL:LIQ |
| Example 7 | NDP-9 | 1720 | 100 | Comparative | 350 |
| | 100 | | | Compound 7 | |
| | | | | 400 | |

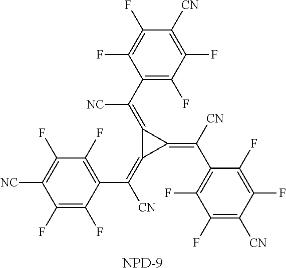

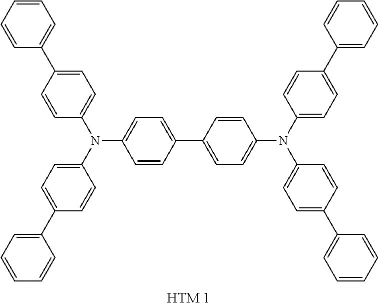 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; "HTM 1"]

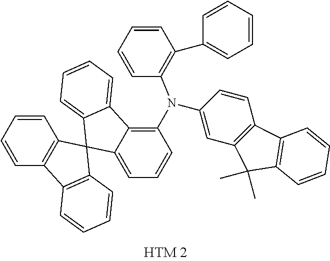 [SMILES: CC1(C)c2ccccc2-c2ccc(N(c3ccccc3-c3ccccc3)c3cccc4c3-c3ccccc3C43c4ccccc4-c4ccccc43)cc21]

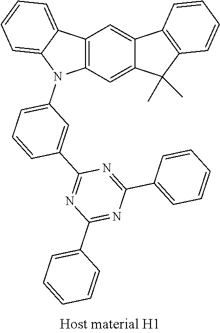 [SMILES: CC1(C)c2ccccc2-c2cc3c4ccccc4n(-c4cccc(-c5nc(-c6ccccc6)nc(-c6ccccc6)n5)c4)c3cc21]

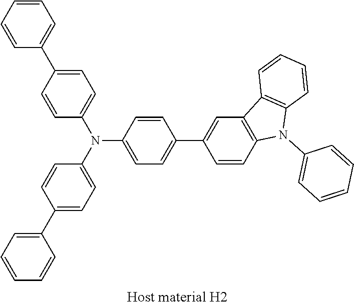 [SMILES: c1ccc(-c2ccc(N(c3ccc(-c4ccccc4)cc3)c3ccc(-c4ccc5c(c4)c4ccccc4n5-c4ccccc4)cc3)cc2)cc1 ; "Host material H2"]

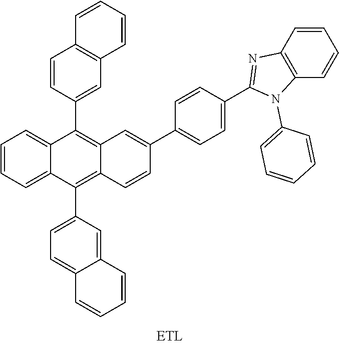 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

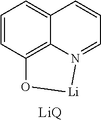

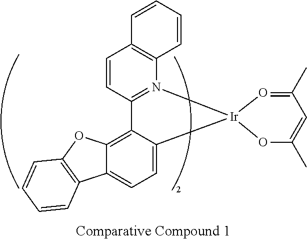

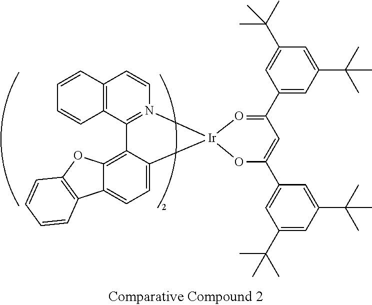

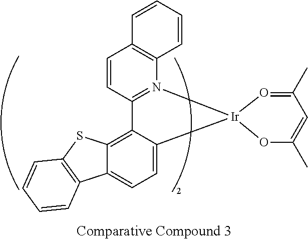

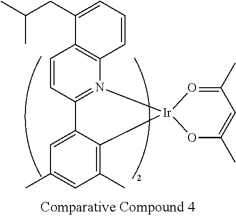

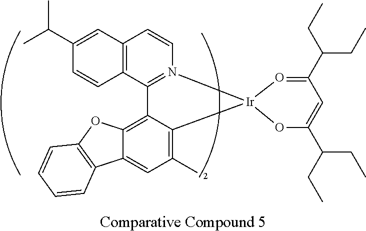

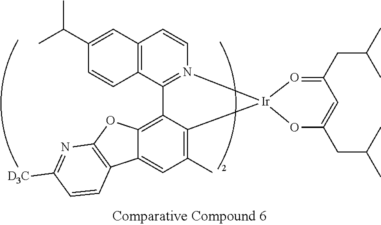

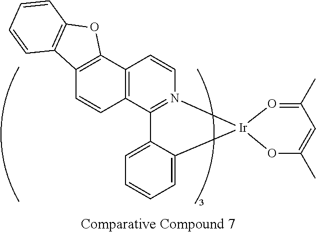

Evaluation: The above-mentioned devices were tested for device performance. In each of the examples and comparative examples, by using a constant current power supply (Keithley 2400), using a fixed current density that flowed through the luminous element, and using a spectral radiance luminance meter (CS 2000), luminescence spectrum was tested. In addition, the voltage value was measured, and the time when the test brightness was 90% of the initial brightness (LT90) was measured. The results were as follows: the current efficiency and device life were both calculated based on the value of Comparative Compound 5 as 100%.

| | | Current | Color | |
|---|---|---|---|---|
| | Starting | efficiency | coordinates | |
| | voltage @ 20 | @20 | @20 mA/cm2 | LT90@ |
| | mA/cm2 V | mA/cm2 | CIEx, CIEy | 8000 nits |
| Example A1 | 4.23 | 132 | 0.701, 0.298 | 130 |
| Example A2 | 4.19 | 136 | 0.701, 0.299 | 138 |
| Example A3 | 4.18 | 139 | 0.702, 0.296 | 133 |
| Example A4 | 4.21 | 140 | 0.703, 0.296 | 136 |
| Example A5 | 4.25 | 128 | 0.702, 0.297 | 139 |
| Example A6 | 4.24 | 130 | 0.702, 0.297 | 134 |
| Example A7 | 4.21 | 143 | 0.702, 0.298 | 152 |
| Example A8 | 4.20 | 139 | 0.701, 0.298 | 159 |
| Example A9 | 4.19 | 139 | 0.702, 0.297 | 161 |
| Example A10 | 4.15 | 145 | 0.701, 0.299 | 137 |
| Example A11 | 4.16 | 146 | 0.702, 0.298 | 135 |
| Example A12 | 4.21 | 137 | 0.703, 0.297 | 132 |
| Example A13 | 4.22 | 138 | 0.703, 0.296 | 133 |
| Example A14 | 4.22 | 138 | 0.702, 0,298 | 135 |
| Comparative | 5.23 | 75 | 0.700, 0.299 | 51 |
| Example 1 | | | | |
| Comparative | 5.15 | 72 | 0.701, 0.298 | 50 |
| Example 2 | | | | |
| Comparative | 5.34 | 74 | 0.703, 0.296 | 42 |
| Example 3 | | | | |
| Comparative | 5.52 | 63 | 0.702, 0.297 | 37 |
| Example 4 | | | | |
| Comparative | 4.88 | 100 | 0.701, 0.298 | 100 |
| Example 5 | | | | |
| Comparative | 4.74 | 95 | 0.702, 0,298 | 118 |
| Example 6 | | | | |
| Comparative | 4.49 | 118 | 0.701, 0.298 | 120 |
| Example 7 | | | | |

From the comparison of the data in the above table, it can be seen that among devices with the same color coordinates, the organic electroluminescent devices in which the compound of the present disclosure is used as a doping agent all exhibit superior performance in terms of driving voltage, luminous efficiency and device life as compared with the comparative compounds.

Comparison of emission wavelengths in a dichloromethane solution: it was defined as follows: the corresponding compound was prepared into a 10⁻⁵ mol/L solution with dichloromethane, the emission wavelength thereof was measured by means of HITACH F2700 fluorescence spectrophotometer, and the wavelength at the maximum emission in the emission peak was derived. The test results were as follows:

| | PL peak |
|---|---|
| Material | wavelength/nm |
| Ir(La001)₂(Lb005) | 626 |
| Ir(La005)₂(Lb005) | 628 |
| Ir(La007)₂(Lb005) | 629 |
| Ir(La011)₂(Lb005) | 627 |
| Ir(La014)₂(Lb005) | 627 |
| Ir(La026)₂(Lb008) | 627 |
| Ir(La041)₂(Lb031) | 625 |
| Ir(La052)₂(Lb005) | 629 |
| Ir(La052)₂(Lb008) | 630 |
| Ir(La078)₂(Lb005) | 631 |
| Ir(La078)₂(Lb008) | 632 |
| Ir(La078)(Lb005)(Lc004) | 629 |
| Ir(La078)(Lb005) (Lc024) | 629 |
| Ir(La078)2Lb005) (Lc025) | 629 |
| Comparative | 610 |
| Compound 1 | |
| Comparative | 637 |
| Compound 2 | |
| Comparative | 611 |
| Compound 3 | |
| Comparative | 608 |
| Compound 4 | |
| Comparative | 616 |
| Compound 5 | |
| Comparative | 626 |
| Compound 7 | |

From the comparison of the data in the above table, it can be seen that the metal iridium complexes of the present disclosure has a larger red shift as compared with the comparative compounds, and can meet the requirements of industrialization for deep red light, particularly the color gamut of BT2020.

Compared with the prior art, the present disclosure unexpectedly provides a better device luminous efficiency and improved lifetime, as well as a lower sublimation temperature and more saturated red luminescence through a special collocation of substituents. The above results indicate that the compound of the present disclosure has the advantages of a high optical and electrochemical stability, a high color saturation, a high luminous efficiency, a long device life, etc., and can be used in organic electroluminescent devices. In particular, as a red luminescent dopant, the compound of the present disclosure has the potential to be applied to the OLED industry, particularly for display, lighting and automobile taillights.

1. A metal iridium compound having a general formula of Ir(La)(Lb)(Lc), wherein La is a structure shown in formula (1), 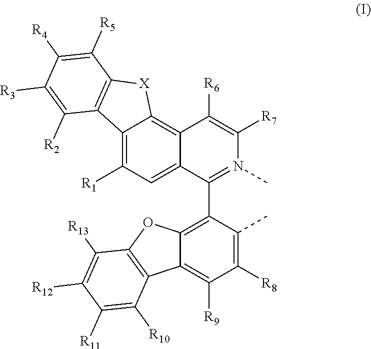
wherein a dotted line represents a position of connection to the metal Ir;
wherein X is O, S, Se, C(R₀)₂, or Si(R₀)₂;
wherein R₀-R₁₃ are each independently hydrogen, deuterium, halogen, cyano, substituted or unsubstituted C1-C10 alkyl, substituted or unsubstituted C1-C10 heteroalkyl, substituted or unsubstituted C3-C20 cycloalkyl, substituted or unsubstituted C3-C20 heterocycloalkyl, substituted or unsubstituted C2-C10 alkenyl, substituted or unsubstituted C2-C10 alkynyl, substituted or unsubstituted C6-C30 aryl, substituted or unsubstituted C2-C30 heteroaryl, substituted or unsubstituted tri(C1-C10)alkylsilyl, substituted or unsubstituted tri(C6-C12)arylsilyl, substituted or unsubstituted di(C1-C10)alkylmono(C6-C30)arylsilyl, or substituted or unsubstituted mono(C1-C10)alkyldi(C6-C30)arylsilyl, or two adjacent groups of R₁₀-R₁₃ are connected with each other to form an aliphatic ring;
wherein R₈ is not hydrogen, deuterium, halogen, or cyano;
wherein the heteroalkyl, heterocycloalkyl, and heteroaryl contain at least one O, N or S heteroatom;
wherein the substitution is substitution with deuterium, F, Cl, Br, C1-C6 alkyl, C3-C6 cycloalkyl, C1-C6 alkylamino, cyano, isocyano or phosphine group, and a number of substitutions is from mono-substitution to the maximum number of substitutions;
wherein Lb is a structure shown in formula (2), 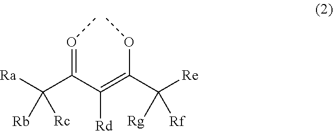
wherein a dotted line represents a position of connection to the metal Ir;
wherein Ra-Rg are each independently hydrogen, deuterium, halogen, substituted or unsubstituted C1-C10 alkyl, substituted or unsubstituted C3-C20 cycloalkyl, substituted or unsubstituted C1-C10 heteroalkyl, or substituted or unsubstituted C3-C20 heterocycloalkyl, or Ra, Rb, and Rc are connected in pairs to form an aliphatic ring, and Re, Rf, and Rg are connected in pairs to form an aliphatic ring;
wherein the heteroalkyl and heterocycloalkyl contain at least one O, N or S heteroatom;
wherein the substitution is substitution with deuterium, F, Cl, Br, C1-C4 alkyl, C1-C4 alkoxy, C3-C6 cycloalkyl, C1-C4 alkylamino, cyano, isocyano, or phosphine group;
wherein Lc is a single anionic bidentate ligand, and Lc is different from Lb and is not an OO-type ligand;
wherein Lc and La are the same or different, the difference means that a mother nucleus structure is different, or the mother nucleus structure is the same but substituents are different, or the mother nucleus structure is the same and the substituents are the same but positions of the substituents are different;
wherein La, Lb, and Lc are connected in pairs or in triplets to form a multidentate ligand.

2. The metal iridium complex according to claim 1, wherein X is O, S, C(R₀)₂, or Si(R₀)₂, and R₀ is substituted or unsubstituted C1-C6 alkyl.

3. The metal iridium complex according to claim 2, wherein at least one of R₂-R₇ is not H.

4. The metal iridium complex according to claim 3, wherein at least one of R₁-R₇ is F, cyano, substituted or unsubstituted C1-C6 alkyl, or substituted or unsubstituted C3-C10 cycloalkyl, and the substitution is substitution with deuterium, F, C1-C5 alkyl, or C3-C6 cycloalkyl.

5. The metal iridium complex according to claim 1, wherein R₈ is substituted or unsubstituted C1-C6 alkyl, or substituted or unsubstituted C3-C6 cycloalkyl, and the substitution is substitution with deuterium, F, C1-C5 alkyl, or C3-C6 cycloalkyl.

6. The metal iridium complex according to claim 5, wherein R₈ is methyl or deuterated methyl.

7. The metal iridium complex according to claim 1, wherein R₉-R₁₃ are hydrogen.

8. The metal iridium complex according to claim 1, wherein Lc is different from La.

9. The metal iridium complex according to claim 8, wherein Lc is a structure shown in formula (3), 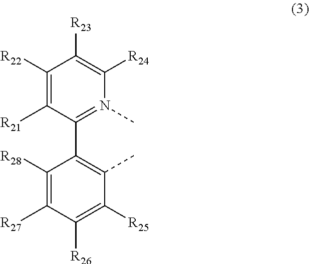
wherein R₂₁-R₂₂ are each independently hydrogen, deuterium, halogen, cyano, hydroxyl, amino, imino, substituted or unsubstituted C1-C10 alkyl, substituted or unsubstituted C1-C10 heteroalkyl, substituted or unsubstituted C3-C20 cycloalkyl, substituted or unsubstituted C2-C10 alkenyl, substituted or unsubstituted C2-C10 alkynyl, substituted or unsubstituted C6-C18 aryl, substituted or unsubstituted C2-C17 heteroaryl, substituted or unsubstituted tri(C1-C10)alkylsilyl, substituted or unsubstituted tri(C6-C12)arylsilyl, substituted or unsubstituted di(C1-C10)alkylmono(C6-C30)arylsilyl, or substituted or unsubstituted mono(C1-C10)alkyldi(C6-C30)arylsilyl;
wherein at least two of R₂₅-R₂₈ are not hydrogen;
wherein an aromatic ring shown in the following formula (4) is formed between at least one group of two adjacent groups of R₂₁-R₂₄; 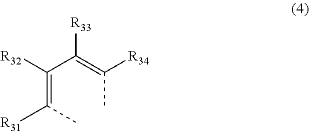
in formula (4),
wherein a dotted line represents a position of connection to a pyridine ring;
wherein R₃₁-R₃₄ are each independently hydrogen, deuterium, halogen, cyano, substituted or unsubstituted C1-C10 alkyl, substituted or unsubstituted C1-C10 heteroalkyl, substituted or unsubstituted C3-C20 cycloalkyl, substituted or unsubstituted C2-C10 alkenyl, substituted or unsubstituted C2-C10 alkynyl, substituted or unsubstituted C6-C18 aryl, substituted or unsubstituted C2-C17 heteroaryl, substituted or unsubstituted tri(C1-C10)alkylsilyl, substituted or unsubstituted tri(C6-C12)arylsilyl, substituted or unsubstituted di(C1-C10)alkylmono(C6-C30)arylsilyl, or substituted or unsubstituted mono(C1-C10) alkyldi(C6-C30)arylsilyl, or two adjacent groups of R₃₁-R₃₄ are connected with each other to form an aliphatic ring or an aromatic ring;
wherein the heteroalkyl and heteroaryl contain at least one O, N or S heteroatom;
wherein the substitution is substitution with deuterium, F, Cl, Br, C1-C6 alkyl, C3-C6 cycloalkyl, C1-C6 alkylamino, cyano, isocyano or phosphine group, and a number of substitutions is from mono-substitution to the maximum number of substitutions.

10. The metal iridium complex according to claim 9, wherein an aromatic ring shown in formula (4) is formed between at least one group of two adjacent groups of R₂₁-R₂₃, R₃₁-R₃₄ are each independently hydrogen, deuterium, halogen, cyano, substituted or unsubstituted C1-C6 alkyl, substituted or unsubstituted C1-C6 heteroalkyl, substituted or unsubstituted C3-C10 cycloalkyl, substituted or unsubstituted C6-C10 aryl, and substituted or unsubstituted C2-C10 heteroaryl.

11. The metal iridium compound according to claim 10, wherein Lc is one of the following structural formulas, or one of the following structural formulas in which the hydrogen atom(s) is/are partially or completely deuterated, or one of the following structural formulas in which the hydrogen atom(s) is/are partially or completely fluorinated,

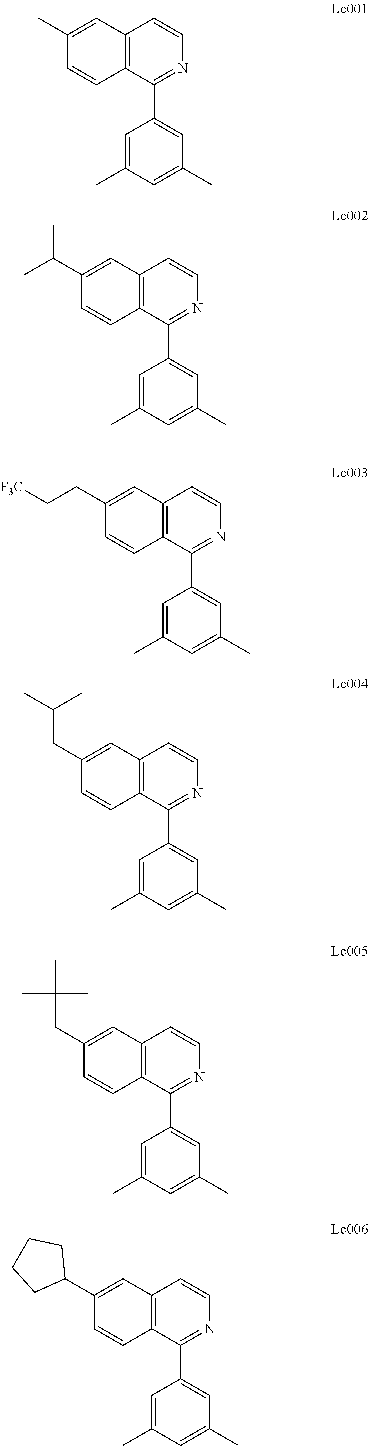

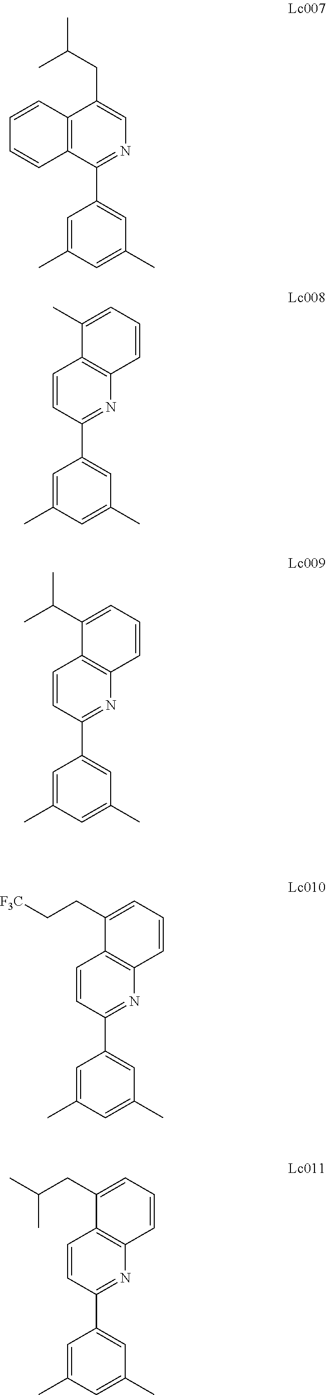

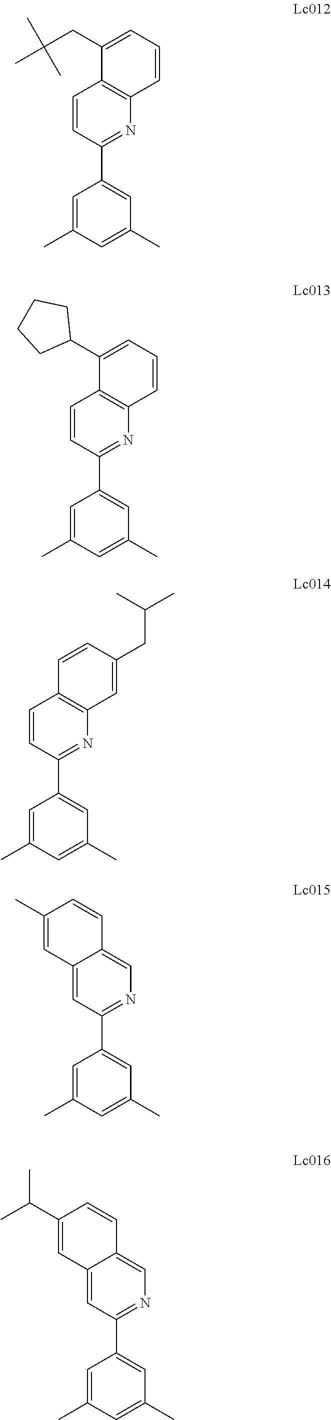

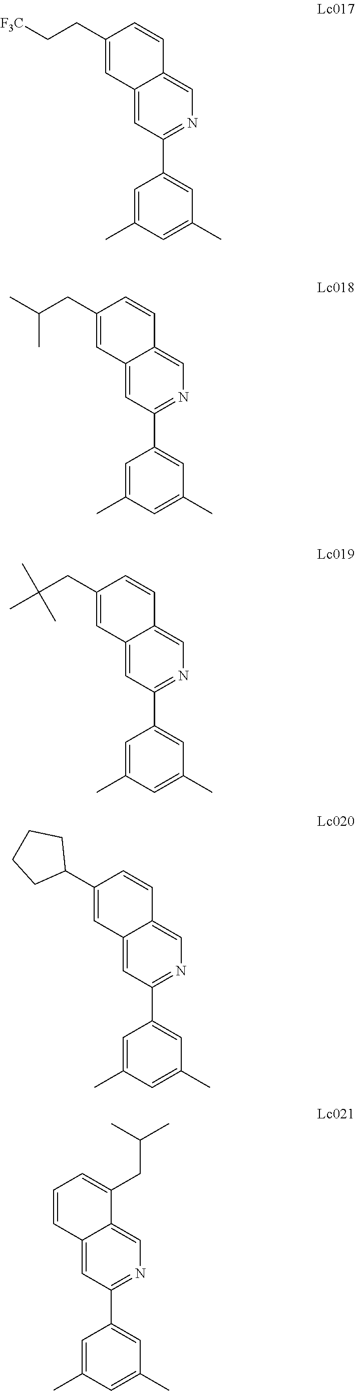

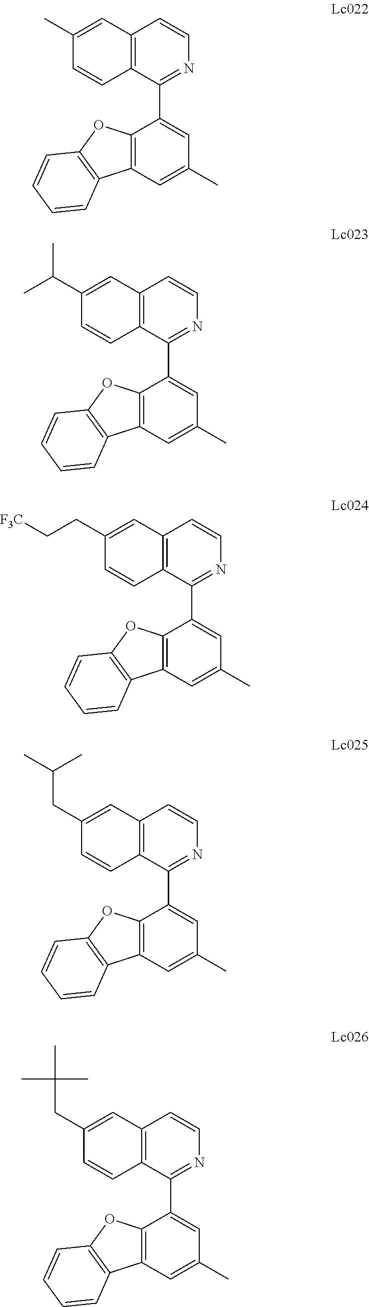

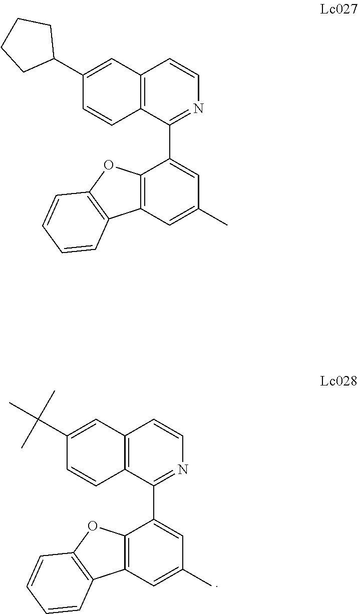

12. The metal iridium compound according to claim 3, wherein La is one of the following structural formulas, or one of the following structural formulas in which the hydrogen atom(s) is/are partially or completely deuterated, or one of the following structural formulas in which the hydrogen atom(s) is/are partially or completely fluorinated,

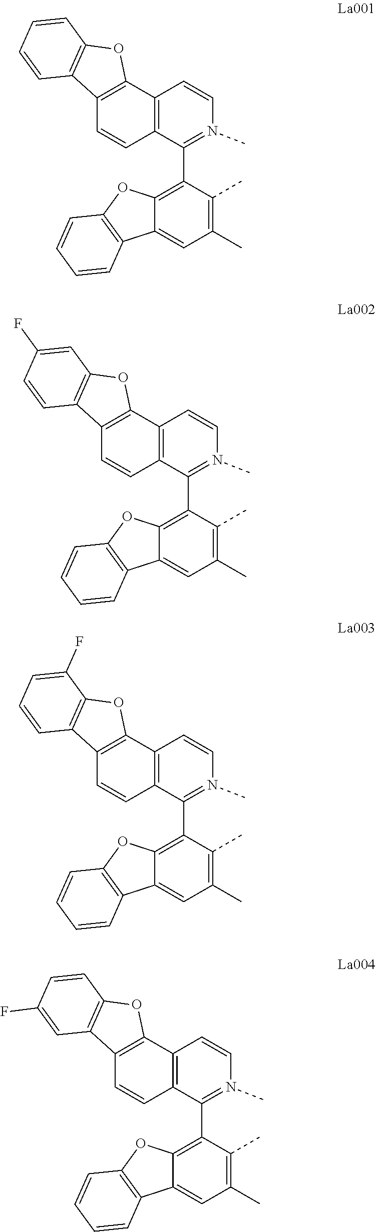

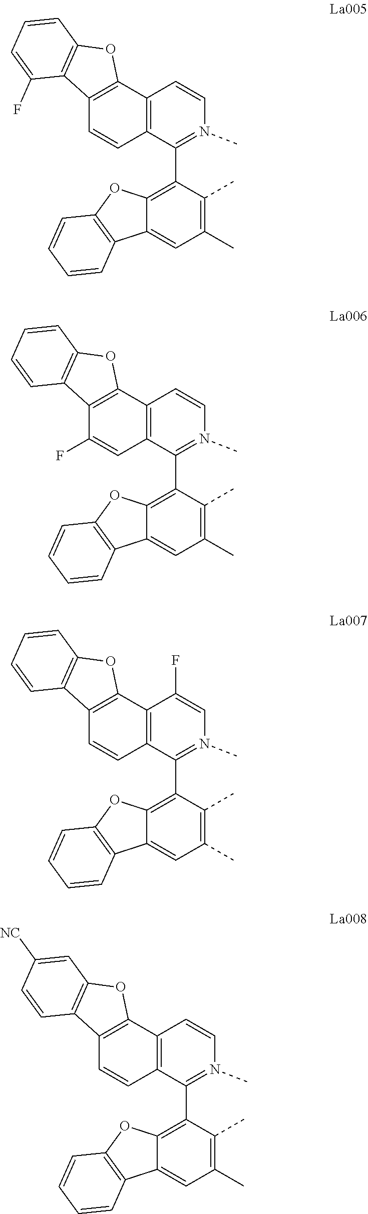

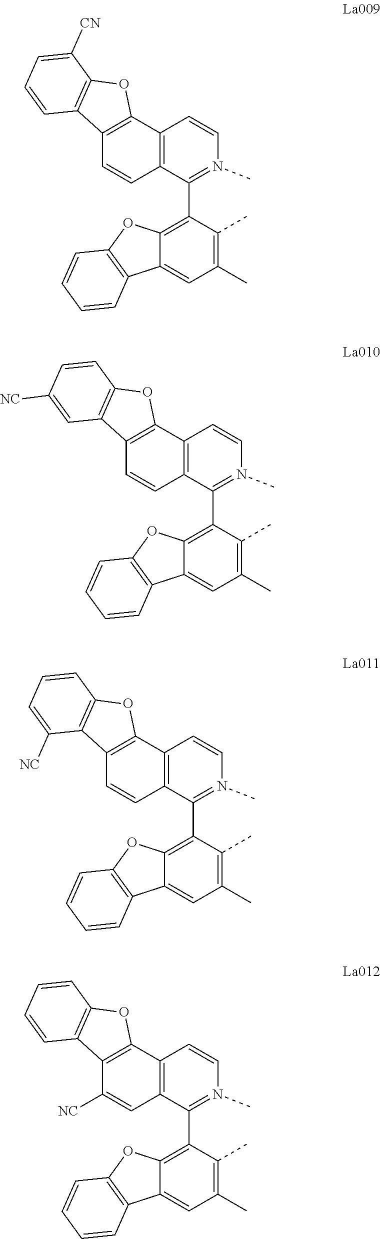

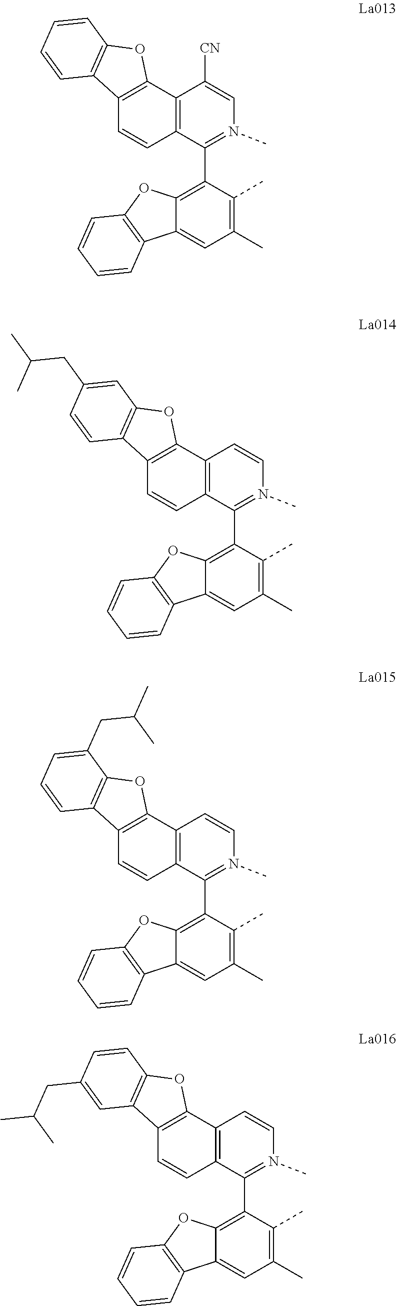

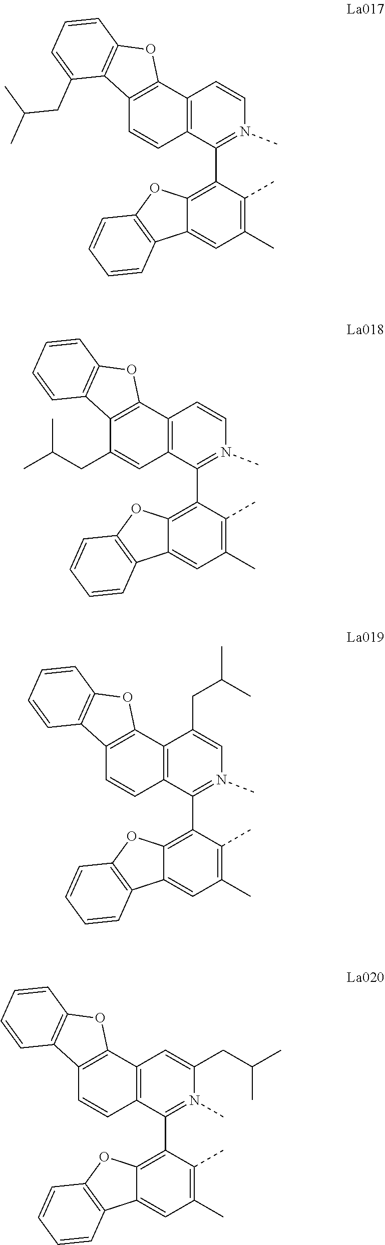

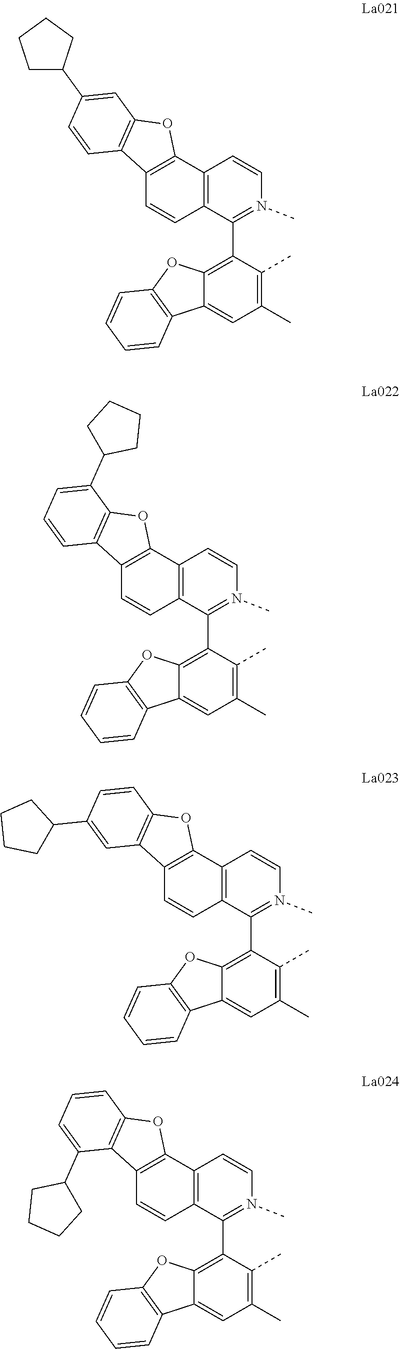

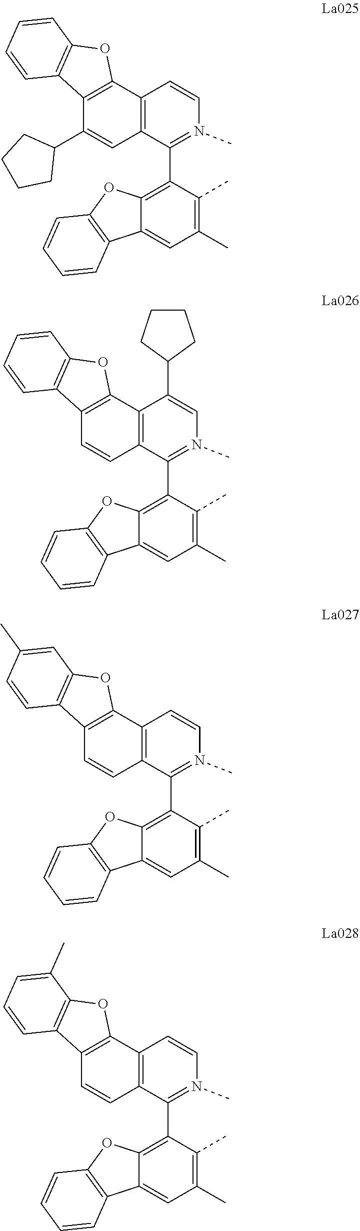

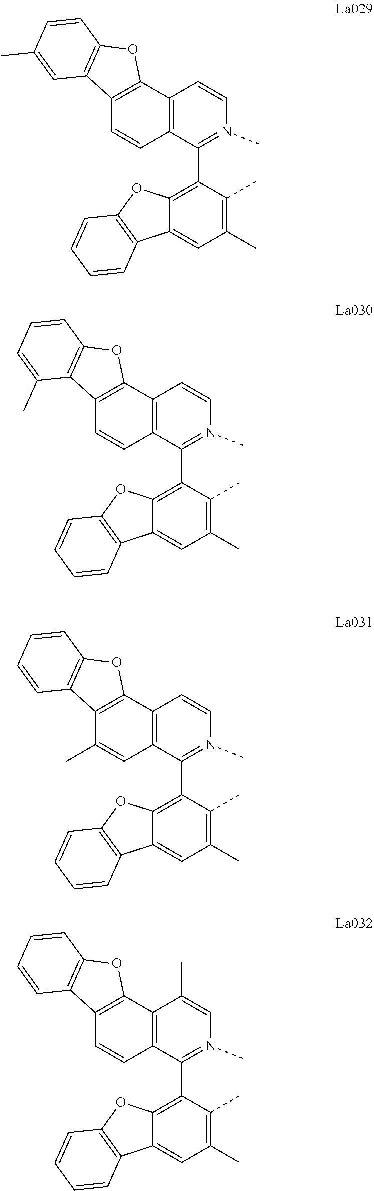

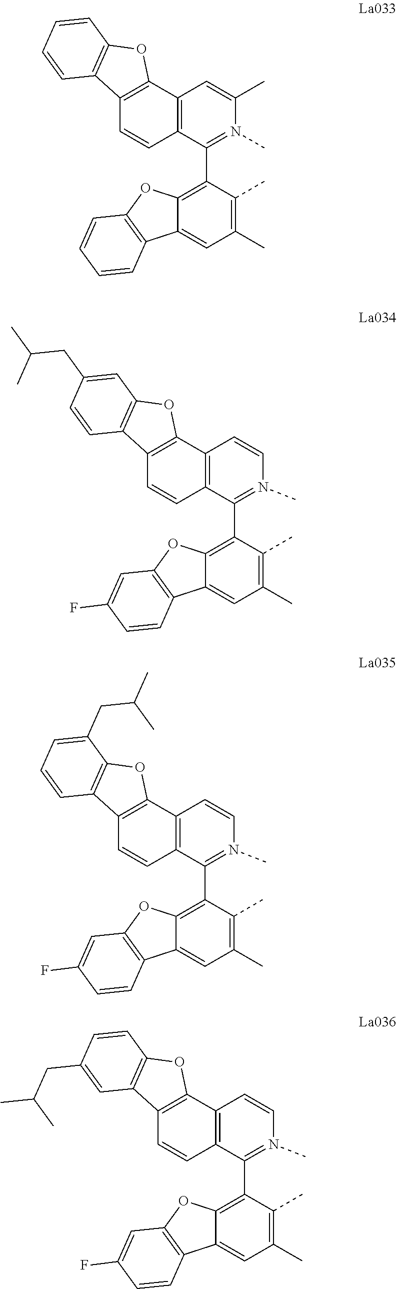

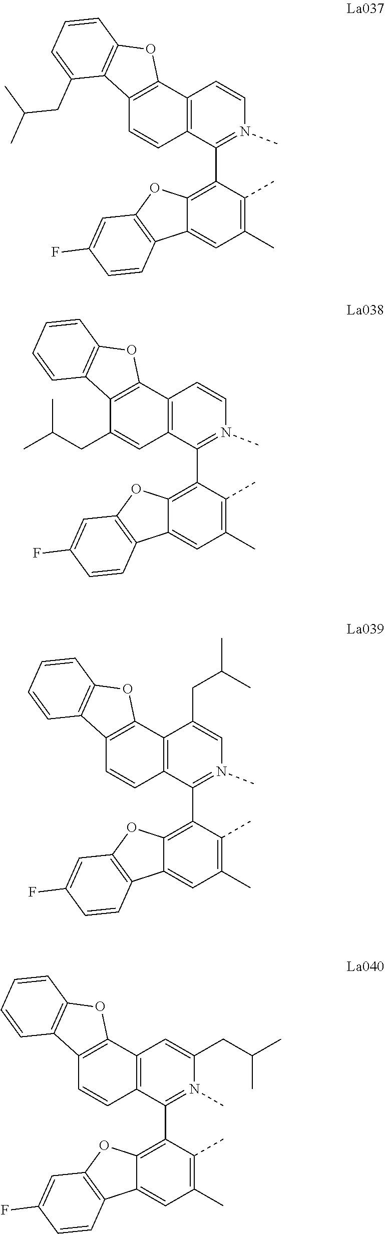

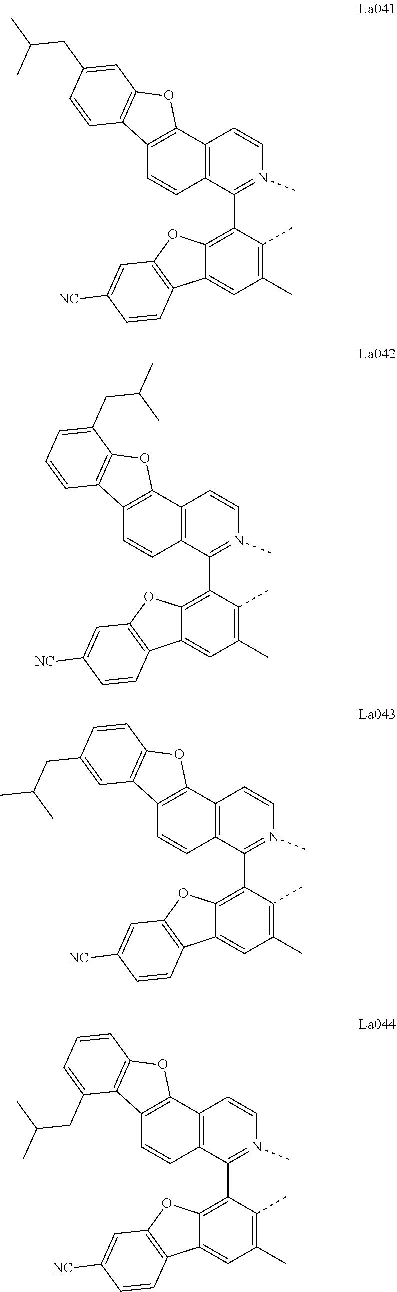

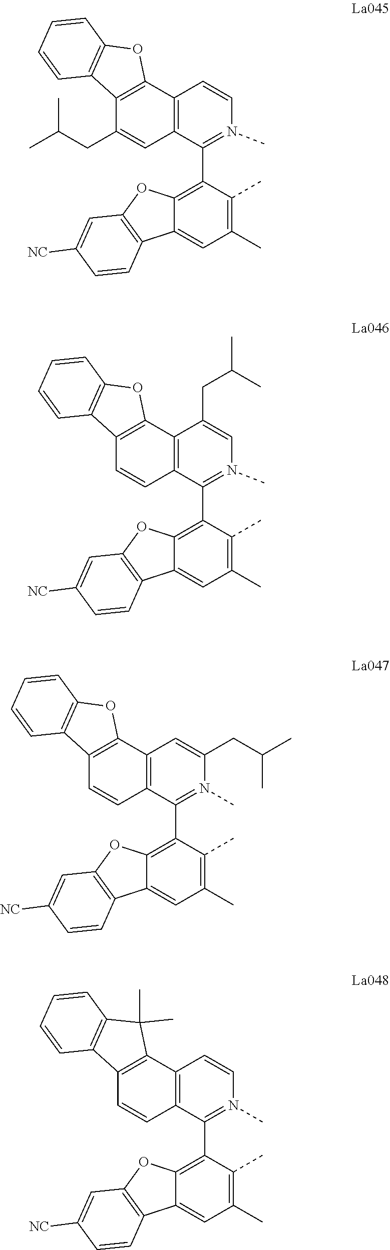

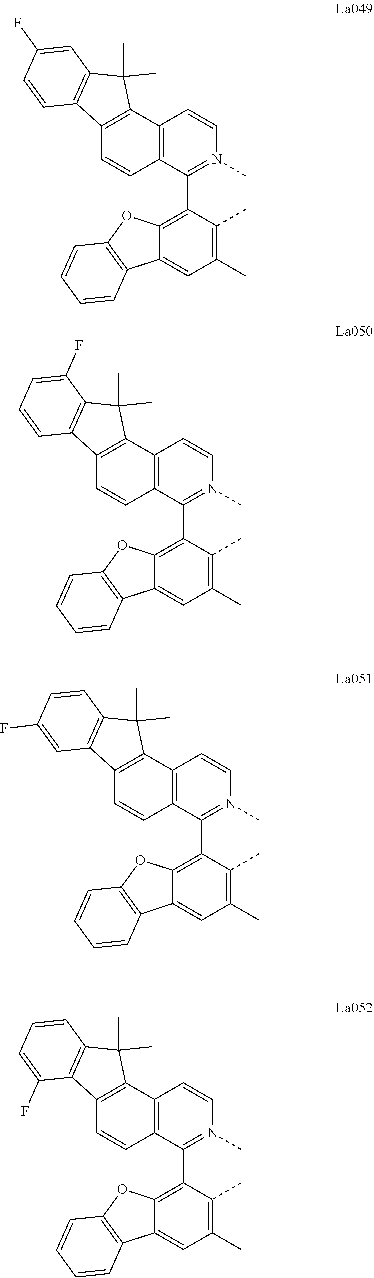

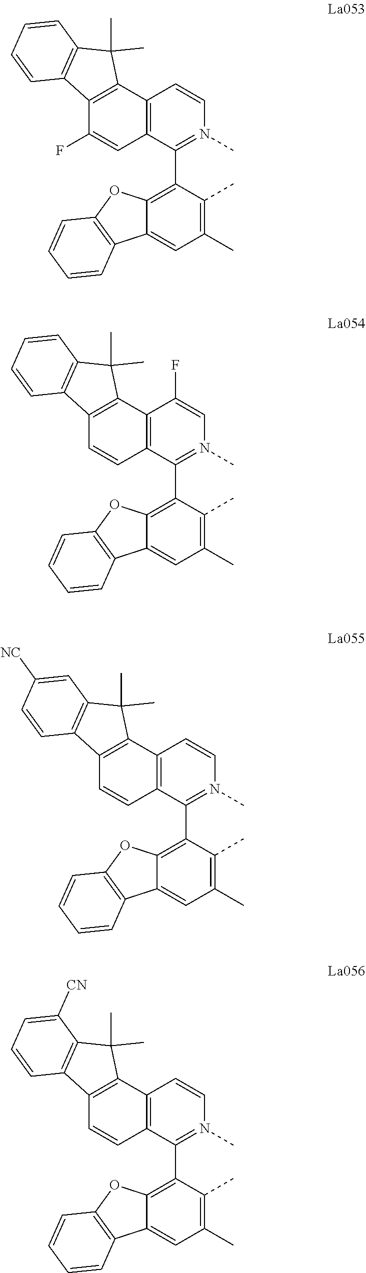

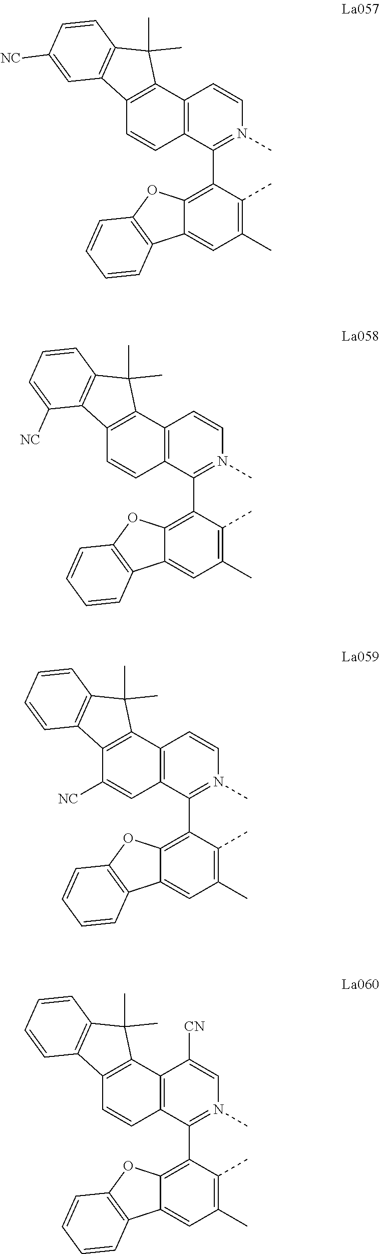

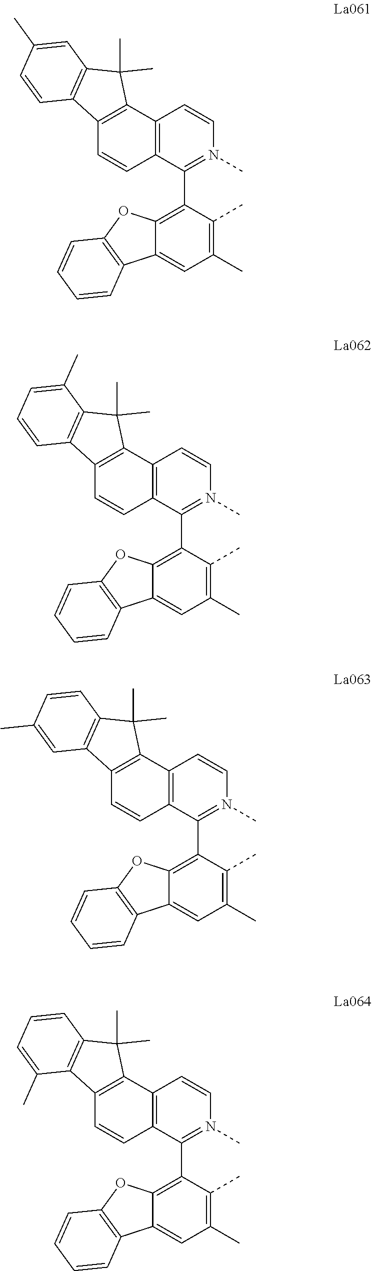

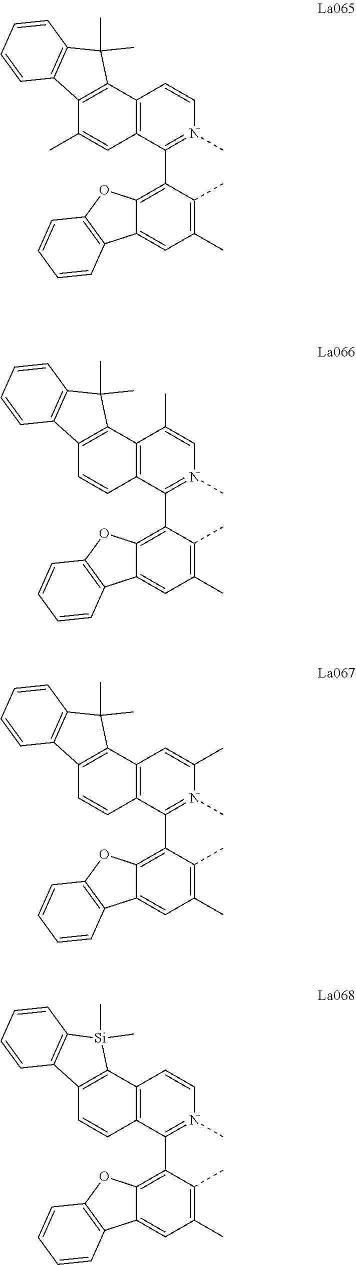

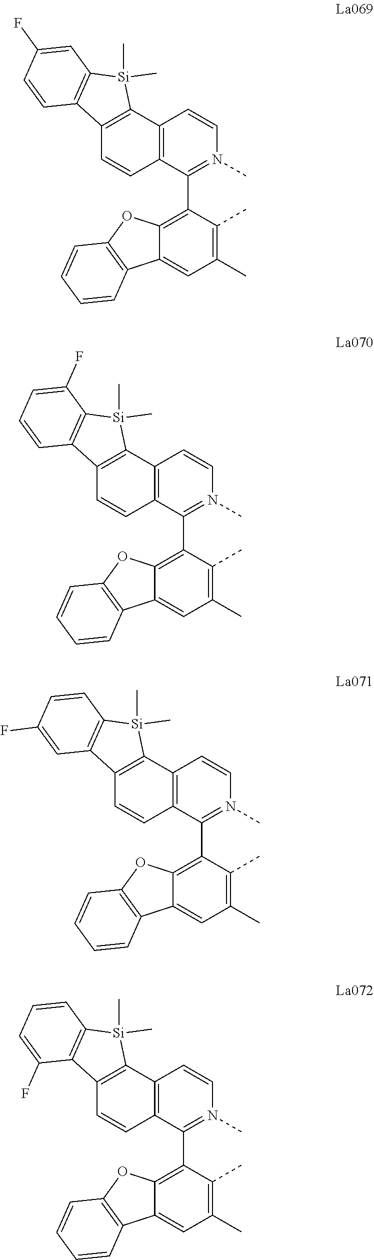

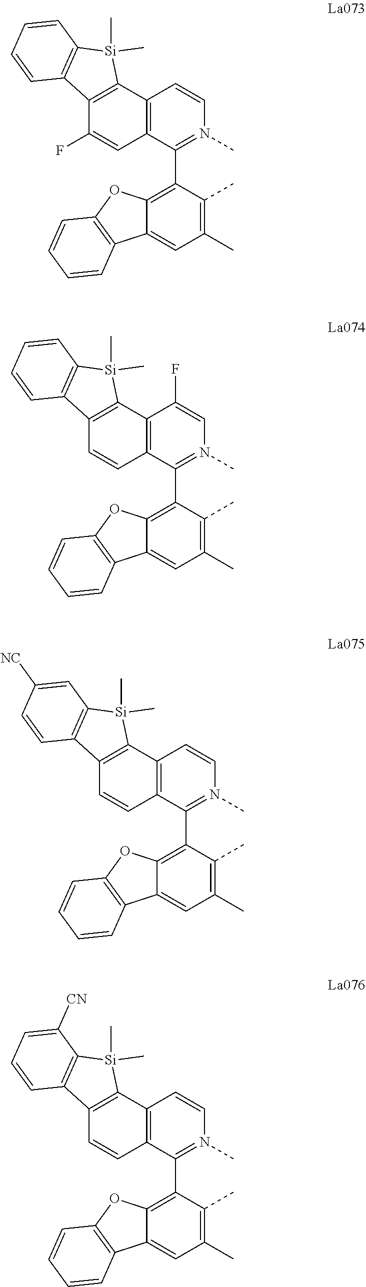

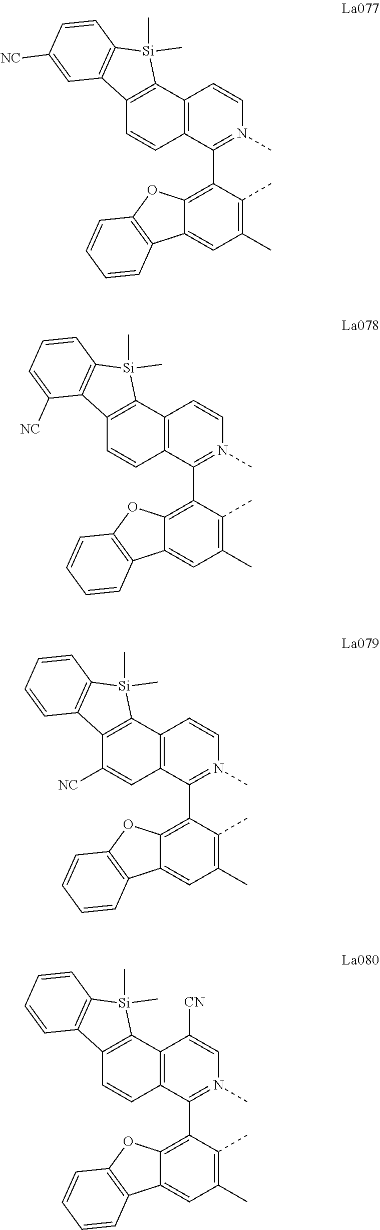

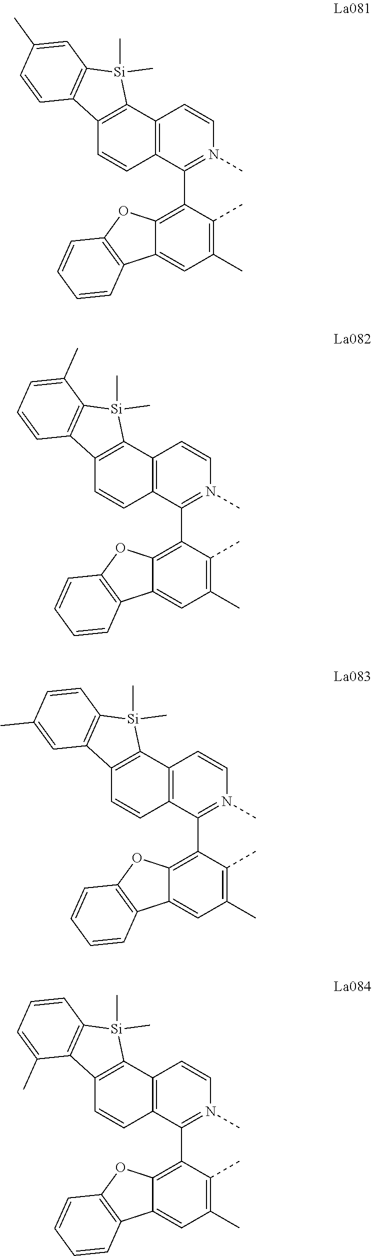

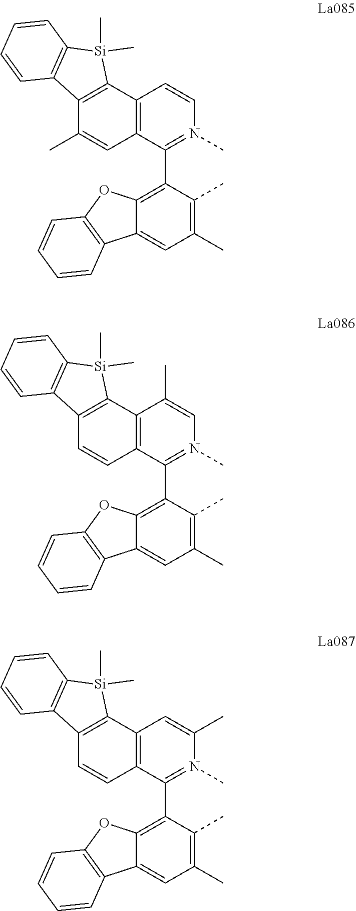

13. The metal iridium compound according to claim 3, wherein Lb is one of the following structural formulas, or one of the following structural formulas in which the hydrogen atom(s) is/are partially or completely deuterated, or one of the following structural formulas in which the hydrogen atom(s) is/are partially or completely fluorinated,

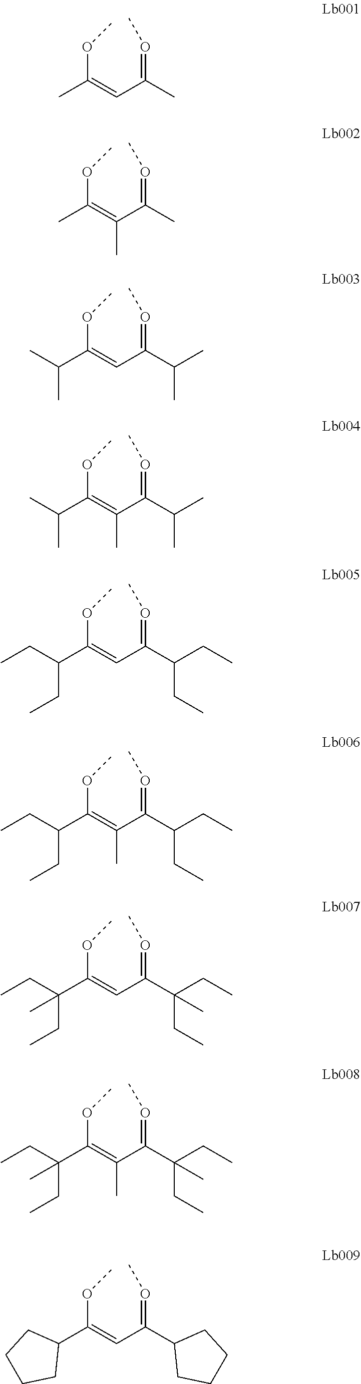

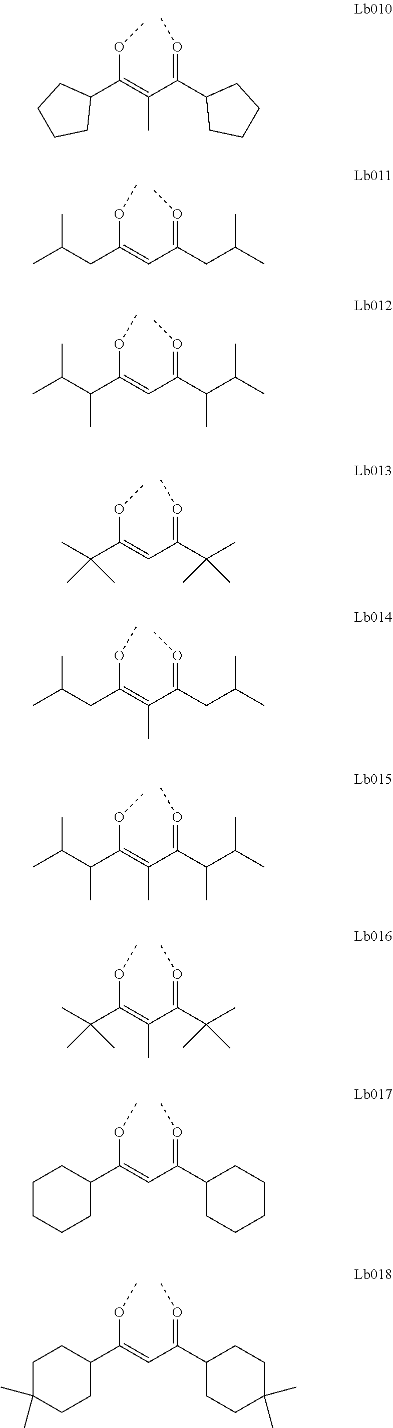

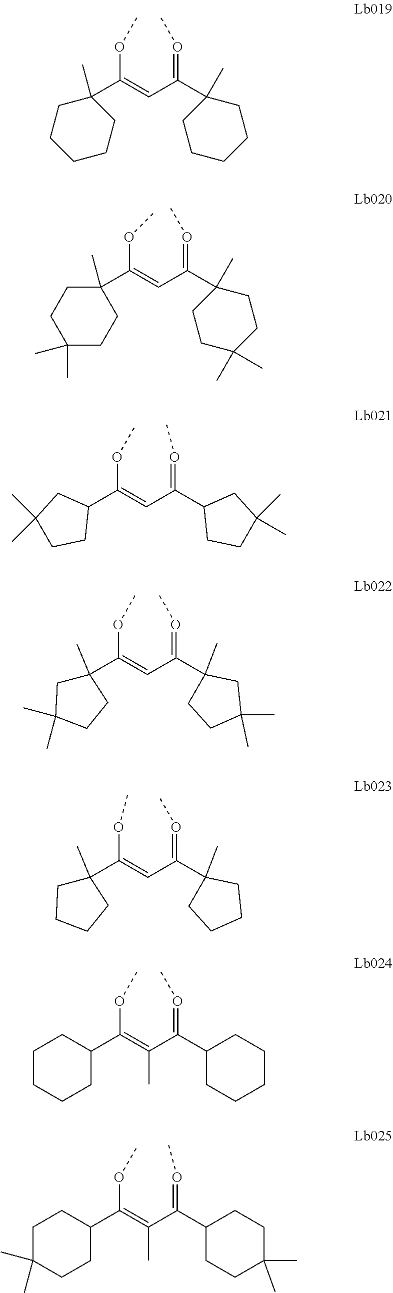

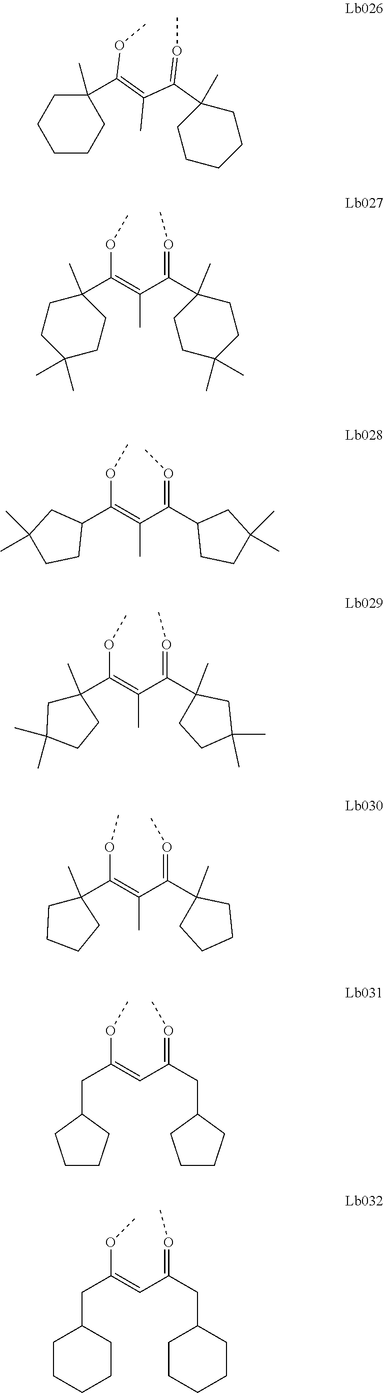

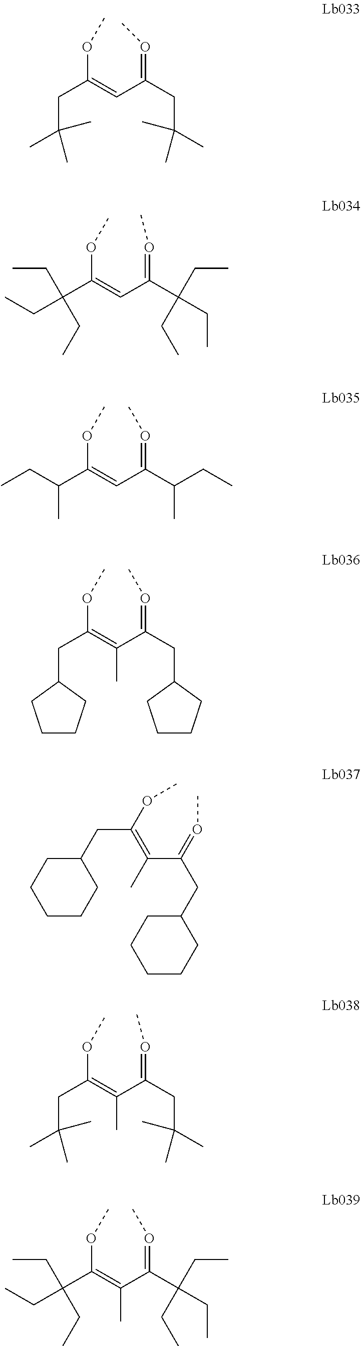

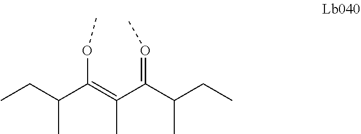

14. An electroluminescent device comprising a cathode, an anode, and an organic layer arranged between the cathode and the anode, wherein the organic layer comprises the metal iridium complex according to claim 1.

15. The electroluminescent device according to claim 14, wherein the organic layer comprises a luminous layer, and the luminous layer comprises the metal iridium complex used as a red luminescent doping material.

16. A ligand La, having a structural formula shown as below: 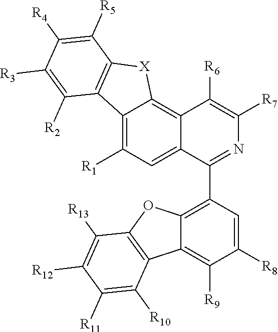
wherein R₁-R₁₃ and X are as defined in claim 1.

17. The electroluminescent device according to claim 14, wherein the organic layer comprises a hole injection layer, and the hole injection layer comprises the metal iridium complex used as a hole injection material.

18. A ligand La, having a structural formula shown as below: 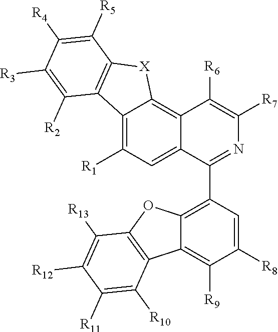
wherein R₁-R₁₃ and X are as defined in claim 2.

19. A ligand La, having a structural formula shown as below: 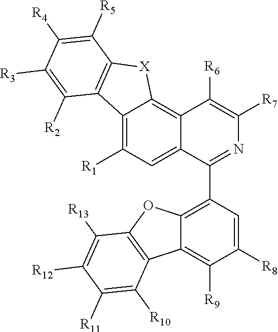
wherein R₁-R₁₃ and X are as defined in claim 3.

20. A ligand La, having a structural formula shown as below: 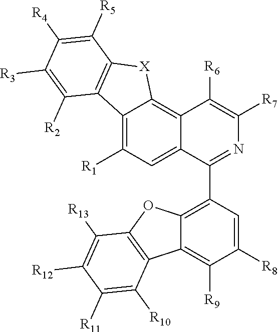
wherein R₁-R₁₃ and X are as defined in claim 4.
